(19) United States
(12) Patent Application Publication
(10) Pub. No.: US 2025/0295598 A1
(43) Pub. Date: Sep. 25, 2025

(54) MULTICOMPONENT CAPSULES

(71) Applicant: Natals, Inc., Culver City, CA (US)

(72) Inventors: Nima ALAMDARI, Los Angeles, CA (US); Shilpa A. RAUT, Long Beach, CA (US); Mastaneh SHARAFI, Redondo Beach, CA (US); Katerina SCHNEIDER, Los Angeles, CA (US); Arianne Y. VANCE, Santa Monica, CA (US)

(21) Appl. No.: 18/886,517

(22) Filed: Sep. 16, 2024

Related U.S. Application Data
(60) Provisional application No. 63/569,020, filed on Mar. 22, 2024.

(51) Int. Cl. A61K 9/48; A61J 1/14; A61K 9/50; A61K 31/07; A61K 31/12; A61K 31/198; A61K 31/375; A61K 31/522; A61K 33/06; A61K 33/26; A61K 36/15; A61K 36/185; A61K 36/258; A61K 36/28; A61K 36/41; A61K 36/73; A61K 36/77; A61K 36/81; A61K 36/88; A61K 36/889; A61K 36/899; A61P 25/20

(52) U.S. Cl. CPC A61K 9/4808; A61J 1/1412; A61K 9/4866; A61K 9/5036; A61K 9/5084; A61K 31/07; A61K 31/12; A61K 31/198; A61K 31/375; A61K 31/522; A61K 33/06; A61K 33/26; A61K 36/15; A61K 36/185; A61K 36/258; A61K 36/28; A61K 36/41; A61K 36/73; A61K 36/77; A61K 36/81; A61K 36/88; A61K 36/889; A61K 36/899; A61P 25/20

(57) ABSTRACT

The present disclosure provides a solid dosage form for customized release of a nutraceutical, wherein the solid dosage form comprises a plurality of tablets in a single capsule, wherein at least one tablet of the plurality comprises a dose of the nutraceutical. Also provided herein are methods for improving bioavailability of a nutraceutical in a subject by administering a solid dosage form described herein.

### CROSS REFERENCE TO RELATED APPLICATIONS

This application claims priority to U.S. Provisional Application No. 63/569,020 filed on Mar. 22, 2024, the contents of which are incorporated herein by reference in its entirety.

### BACKGROUND

The production and consumption of nutraceuticals and food supplements in solid dosage form (e.g., tablets, gummies, capsules) is increased, in part due to the demand by consumers to supplement diet and improve overall health and emotional well-being. Solid dosage forms are generally preferred due to the ease of packaging, shelf-stability, ease of and administration (i.e., usually orally). However, many nutraceuticals are manufactured in simple solid dosage forms, such as instant release dry-filled capsules, extended release tablets, or gummies, regardless of the optimal delivery method required to target a specific benefit. As a result, these nutraceuticals may have poor bioavailability, ineffective targeting, and low gastrointestinal permeability. Additionally, simple solid dosage forms may result in a mismatch between the delivery of the nutraceutical and endogenous metabolic or physiological rhythms.

Accordingly, there remains a need for improved delivery formulations to optimize delivery of nutraceuticals.

### SUMMARY

In some aspects, the present disclosure provides a customized-release nutraceutical capsule comprising two or more spherical tablets, wherein each spherical tablet comprises a coating and a matrix, wherein the matrix comprises a dose of a nutraceutical, and wherein the matrix is substantially encapsulated by the coating, wherein at least one of the spherical tablets is an extended release tablet, wherein the matrix and the coating of the extended release tablet each comprise a rate controlling polymer in an amount sufficient to release the dose over an extended duration of time, and wherein the spherical tablets are enclosed in the capsule.

In some embodiments of the foregoing or related aspects, the capsule comprises 3, 4, or 5 spherical tablets. In some embodiments, the capsule comprises 3 spherical tablets. In some embodiments, at least two of the spherical tablets are extended release tablets.

In some embodiments of the foregoing or related aspects, the amount of the rate controlling polymer in the matrix of the extended release tablet is about 1% to about 30% by weight of the tablet. In some embodiments, the amount of the rate controlling polymer in the matrix of the extended release tablet is about 5% to about 15% by weight of the tablet. In some embodiments, the amount of the rate controlling polymer in the matrix of the extended release tablet is about 2% to about 25% by weight of the tablet. In some embodiments, the amount of the rate controlling polymer in the matrix of the extended release tablet is about 1% to about 15% by weight of the tablet. In some embodiments, the amount of the rate controlling polymer in the matrix of the extended release tablet is about 15% to about 25% by weight of the tablet.

In some embodiments of the foregoing or related aspects, the matrix of the extended release tablet further comprises a diluent, a lubricant, a glidant, and optionally a colorant. In some embodiments, the capsule comprises an amount of the coating of the extended release tablet, wherein the amount is about 0.5% to about 10% by weight of the tablet. In some embodiments, the coating of the extended release tablet further comprises a flow agent and an alginate salt.

In some embodiments of the foregoing or related aspects, at least one of the spherical tablets is an immediate release tablet. In some embodiments, the matrix of the immediate release tablet comprises a disintegrant. In some embodiments, the matrix of the immediate release tablet further comprises a lubricant, a diluent, a glidant, and optionally a colorant. In some embodiments, the coating of the immediate release tablet comprises a second rate controlling polymer. In some embodiments, the capsule comprises an amount of the coating of the immediate release tablet, wherein the amount is about 0.5% to about 10% by weight of the tablet.

In some embodiments of the foregoing or related aspects, the dose of the nutraceutical is about 0.05% to about 80% by weight of the tablet. In some embodiments, the dose of the nutraceutical in each of the spherical tablets is the same or different.

In some embodiments of the foregoing or related aspects, the rate controlling polymer and/or the second rate controlling polymer comprises a cellulose derivative. In some embodiments, the cellulose derivative is selected from hydroxypropylmethylcellulose (HPMC), hydroxypropyl cellulose (HPC), and microcrystalline cellulose (MCC). In some embodiments, the matrix comprises one nutraceutical. In some embodiments, the matrix comprises more than one nutraceutical.

In some embodiments of the foregoing or related aspects, the nutraceutical is selected from a vitamin, a stimulant, a mineral, a plant extract, a prebiotic, a probiotic, a postbiotic, a botanical extract, a botanical oil, a synthetic active, a botanical ingredient, a plant-based ingredient, an amino acid, a nootropic, a nootropic, and a combination thereof. In some embodiments, the nutraceutical comprises iron, vitamin C, vitamin A, a probiotic, or a combination thereof. In some embodiments, the nutraceutical comprises a stimulant, optionally caffeine, guarana, or both. In some embodiments, the nutraceutical comprises caffeine, TeaCrine, L-theanine, or a combination thereof. In some embodiments, the nutraceutical comprises a botanical ingredient, optionally curcumin, ginseng, and/or rhodiola. In some embodiments, the nutraceutical comprises a mineral, optionally calcium, magnesium, or both. In some embodiments, the nutraceutical comprises a non-stimulant nootropic. In some embodiments, the nutraceutical comprises any one or any combination of phosphatidylserine, green oat extract, citicoline, pycnogenol, sceletium tortuosum, bacopa monnieri, Rhodiola rosea, Ginkgo biloba, Panax ginseng, carotenoids including lutein, zeaxanthin, and meso-zeaxanthin, spearmint extract, French grape (Vitis vinifera L) extract, and North-American wild blueberry (Vaccinium angustifolium A.) extract. In some embodiments, the nutraceutical comprises any one or any combination of vitamin C, zinc, vitamin A, oat beta glucan, larch arabinogalactan, Euglena gracilis fermentate, and elderberry. In some embodiments, the nutraceutical comprises any one or any combination of a millet seed extract, an apple extract, a banana flower extract, saw palmetto, spermidine polyamines, and billygoat weed extract. In some embodiments, the nutraceutical comprises any one or any combination of a saffron extract, a melon juice concentrate high in superoxide dismutase (SOD), L-theanine, GABA, lemon balm extract, chamomile extract, curcumin, pomegranate extract, magnesium, carotenoids including lutein, zeaxanthin, and meso-zeaxanthin. In some embodiments, the matrix of at least one of the spherical tablets further comprises a botanical oil. In some embodiments, the nutraceutical is the same for the two or more spherical tablets. In some embodiments, the nutraceutical is different for the two or more spherical tablets.

In some embodiments of any of the foregoing or related aspects, the extended duration of time is about 1 hour to about 12 hours under gastrointestinal dissolution conditions. In some embodiments, the immediate release tablet releases the dose of the nutraceutical in about 5 minutes to about 90 minutes under gastrointestinal dissolution conditions.

In aspects, the disclosure provides a customized-release nutraceutical capsule, comprising three spherical tablets, wherein each spherical tablet comprises a coating and a matrix, wherein the matrix is substantially encapsulated by the coating, and wherein the matrix comprises a dose of melatonin, and wherein the three spherical tablets are enclosed in the capsule. In some embodiments, the three spherical tablets comprise an extended release tablet. In some embodiments, the three spherical tablets comprise two extended release tablets. In some embodiments, the three spherical tablets comprise three extended release tablets. In some embodiments, the three spherical tablets comprise an immediate release tablet and an extended release tablet. In some embodiments, the three spherical tablets comprise one immediate release tablet and two extended release tablets. In some embodiments, the three spherical tablets comprise one immediate release tablet, one intermediate release tablet, and one extended release tablet. In some embodiments, the three spherical tablets each independently comprise a dose of about 0.5 mg to about 5 mg melatonin. In some embodiments, the three spherical tablets each comprise the same dose of melatonin. In some embodiments, the three spherical tablets each comprise a different dose of melatonin.

In some aspects, the disclosure provides a customized-release nutraceutical capsule, comprising three spherical tablets, wherein at least one spherical tablet is an extended release tablet, wherein each spherical tablet comprises a coating and a matrix, wherein the matrix is substantially encapsulated by the coating, and wherein the matrix comprises a dose of melatonin, and wherein the three spherical tablets are enclosed in the capsule. In some embodiments, the dose of melatonin is about 0.5 mg to about 5 mg. In some embodiments, the three spherical tablets further comprise an immediate release tablet. In some embodiments, the immediate release tablet comprises a dose of melatonin of about 0.5 mg to about 5 mg (e.g., about 0.5 mg to about 2 mg). In some embodiments, the three spherical tablets further comprise an intermediate release tablet. In some embodiments, the intermediate release tablet comprises a dose of melatonin of about 0.5 mg to about 5 mg (e.g., about 0.5 mg to about 2 mg, about 1 mg to about 4 mg, or about 2 mg to about 5 mg). In some embodiments, the three spherical tablets comprise the extended release tablet, the intermediate release tablet, and the immediate release tablet. In some embodiments, the extended release tablet is a first extended release tablet and the capsule further comprises a second extended release tablet. In some embodiments, the second extended release tablet comprises a dose of melatonin of about 0.5 mg to about 5 mg (e.g., about 0.5 mg to about 2 mg, about 1 mg to about 4 mg, about 2 mg to about 5 mg). In some embodiments, the three spherical tablets comprise the first extended release tablet, the second extended release tablet, and the immediate release tablet.

In some embodiments of the foregoing or related aspects, (i) the immediate release tablet comprises a dose of about 0.5 mg to about 2 mg of melatonin; (ii) the first extended release tablet comprises a dose of about 2 mg to about 4 mg of melatonin; and/or (iii) the second extended release tablet comprises a dose of about 0.5 mg to about 2 mg of melatonin. In some embodiments, the matrix of the first extended release tablet comprises a rate controlling polymer. In some embodiments, the matrix of the first extended release tablet comprises an amount of the rate controlling polymer, wherein the amount is about 5% to about 15% by weight of the tablet. In some embodiments, the matrix of the second extended release tablet comprises a rate controlling polymer. In some embodiments, the matrix of the second extended release tablet comprises an amount of the rate controlling polymer, wherein the amount is about 5% to about 15% by weight of the tablet.

In some embodiments of the foregoing or related aspects, (i) the immediate release tablet comprises a dose of about 0.5 mg to about 2 mg of melatonin; (ii) the extended release tablet comprises a dose of about 2 mg to about 4 mg of melatonin; and/or (iii) the intermediate release tablet comprises a dose of about 0.5 mg to about 2 mg of melatonin. In some embodiments, the matrix of the extended release tablet comprises a rate controlling polymer. In some embodiments, the matrix of the intermediate release tablet comprises a rate controlling polymer. In some embodiments, the matrix of the extended release tablet comprises an amount of the rate controlling polymer, wherein the amount is about 5% to about 15% by weight of the tablet. In some embodiments, the matrix of the intermediate release tablet comprises an amount of the rate controlling polymer, wherein the amount is about 5% to about 15% by weight of the tablet.

In some embodiments of the foregoing or related aspects, the capsule comprises an amount of the rate controlling polymer, wherein the amount is about 5% to about 15% by weight of the tablet. In some embodiments, the coating of the first extended release tablet comprises a rate controlling polymer. In some embodiments, the first extended release tablet comprises an amount of the coating, wherein the amount is about 2% to about 10% by weight of the tablet. In some embodiments, the matrix of the second extended release tablet comprises a rate controlling polymer. In some embodiments, the matrix of the second extended release tablet comprises an amount of the rate controlling polymer, wherein the amount is about 5% to about 15% by weight of the tablet. In some embodiments, the coating of the second extended release tablet further comprises a rate controlling polymer. In some embodiments, the second extended release tablet comprises an amount of the coating, wherein the amount is about 2% to about 10% by weight of the tablet. In some embodiments, the matrix of the immediate release tablet comprises a disintegrant. In some embodiments, the immediate release tablet comprises an amount of the disintegrant, wherein the amount is about 0.1% to about 5% by weight of the tablet. In some embodiments, at least one of the spherical tablets comprises a colorant. In some embodiments, the three spherical tablets comprise a color that is substantially the same. In some embodiments, the three spherical tablets comprise a color that is substantially different. In some embodiments, the immediate release tablet, the first extended release tablet, and optionally the second extended release tablet each comprise a colorant. In some embodiments, the immediate release tablet, the first extended release tablet, and the second extended release tablet comprise a color that is substantially the same. In some embodiments, the immediate release tablet, the first extended release tablet, and the second release tablet comprise a color that is substantially different. In some embodiments, the immediate release tablet is dark blue, the first extended release tablet is light blue, and the second extended release tablet is white.

In some embodiments of the foregoing or related aspects, the coating of the intermediate release tablet comprises a rate controlling polymer. In some embodiments, the intermediate release tablet comprises an amount of the coating, wherein the amount is about 2% to about 10% by weight of the tablet. In some embodiments, the coating of the extended release tablet further comprises a rate controlling polymer. In some embodiments, the extended release tablet comprises an amount of the coating, wherein the amount is about 2% to about 10% by weight of the tablet. In some embodiments, the matrix of the immediate release tablet comprises a disintegrant. In some embodiments, the immediate release tablet comprises an amount of the disintegrant, wherein the amount is about 0.1% to about 5% by weight of the tablet. In some embodiments, at least one of the spherical tablets comprises a colorant. In some embodiments, the three spherical tablets comprise a color that is substantially the same. In some embodiments, the three spherical tablets comprise a color that is substantially different. In some embodiments, the immediate release tablet, the intermediate release tablet, and the extended release tablet each comprise a colorant. In some embodiments, the immediate release tablet, the intermediate release tablet, and the extended release tablet comprise a color that is substantially the same. In some embodiments, the immediate release tablet, the intermediate release tablet, and the second release tablet comprise a color that is substantially different. In some embodiments, the immediate release tablet is dark blue, the intermediate release tablet is light blue, and the extended release tablet is white.

In some embodiments of the foregoing or related aspects, under gastrointestinal dissolution conditions, (i) the immediate release tablet releases the dose in about 0.5 hours to about 1.5 hours; (ii) the first extended release tablet releases the dose in about 4 hours to about 6 hours; and/or (iii) the second extended release tablet releases the dose in about 4 hours to about 6 hours. In some embodiments, under gastrointestinal dissolution conditions, (i) the immediate release tablet releases the dose in about 0.5 hours to about 1.5 hours; (ii) the first extended release tablet releases the dose in about 4 hours to about 8 hours; and/or (iii) the second extended release tablet releases the dose in about 2 hours to about 6 hours. In some embodiments, under gastrointestinal dissolution conditions, (i) the immediate release tablet releases the dose in about 0.5 hours to about 1.5 hours; (ii) the first extended release tablet releases the dose in about 6 hours to about 8 hours; and/or (iii) the second extended release tablet releases the dose in about 2 hours to about 4 hours. In some embodiments, (i) the immediate release tablet releases the dose in about 1 hour; (ii) the first extended release tablet releases the dose in about 6 hours to about 8 hours; and/or (iii) the second extended release tablet releases the dose in about 3 hours.

In some embodiments of the foregoing or related aspects, under gastrointestinal dissolution conditions, (i) the immediate release tablet releases the dose in about 0.5 hours to about 1.5 hours; (ii) the intermediate tablet releases the dose in about 4 hours to about 6 hours; and/or (iii) the extended release tablet releases the dose in about 4 hours to about 6 hours. In some embodiments, under gastrointestinal dissolution conditions, (i) the immediate release tablet releases the dose in about 0.5 hours to about 1.5 hours; (ii) the intermediate release tablet releases the dose in about 2 hours to about 6 hours; and/or (iii) the extended release tablet releases the dose in about 4 hours to about 8 hours. In some embodiments, under gastrointestinal dissolution conditions, (i) the immediate release tablet releases the dose in about 0.5 hours to about 1.5 hours; (ii) the intermediate release tablet releases the dose in about 2 hours to about 4 hours; and/or (iii) the extended release tablet releases the dose in about 6 hours to about 8 hours. In some embodiments, (i) the immediate release tablet releases the dose in about 1 hour; (ii) the intermediate release tablet releases the dose in about 3 hours; and/or (iii) the extended release tablet releases the dose in about 6 hours to about 8 hours.

In some aspects, the disclosure provides a customized-release nutraceutical capsule, comprising three spherical tablets comprising a first extended release tablet, and a second extended release tablet, wherein each spherical tablet comprises a coating and a matrix, wherein the matrix is substantially encapsulated by the coating, and wherein the matrix comprises a dose of a nutraceutical selected from L-theanine, saffron, ashwagandha, and a combination thereof, and wherein the three spherical tablets are enclosed in the capsule. In some embodiments, the three spherical tablets comprise an immediate release tablet.

In some embodiments of the foregoing or related aspects, the immediate release tablet comprises (i) a dose of L-theanine, wherein the dose is about 60 mg to about 100 mg; (ii) a dose of saffron, wherein the dose is about 2 mg to about 10 mg; and/or (iii) a dose of ashwagandha, wherein the dose is about 20 mg to about 40 mg. In some embodiments, the first extended release tablet comprises (i) a dose of L-theanine, wherein the dose is about 60 mg to about 100 mg; (ii) a dose of saffron, wherein the dose is about 8 mg to about 12 mg; and/or (iii) a dose of ashwagandha, wherein the dose is about 20 mg to about 40 mg. In some embodiments, the second extended release tablet comprises (i) a dose of L-theanine, wherein the dose is about 60 mg to about 100 mg; (ii) a dose of saffron, wherein the dose is about 12 mg to about 20 mg; and/or (iii) a dose of ashwagandha, wherein the dose is about 20 mg to about 40 mg. In some embodiments, (i) the immediate release tablet comprises about 60 mg to about 100 mg L-theanine, about 2 mg to about 10 mg saffron, and about 20 mg to about 40 mg ashwagandha; (ii) the first extended release tablet comprises a dose of about 60 mg to about 100 mg L-theanine, about 8 mg to about 12 mg saffron, and about 20 mg to about 40 mg ashwagandha; (iii) the second extended release tablet comprises a dose of about 60 mg to about 100 mg L-theanine, about 15 mg to about 20 mg saffron, and about 20 mg to about 40 mg ashwagandha. In some embodiments, (i) the immediate release tablet comprises about 78 mg L-theanine, about 5 mg saffron, and about 29 mg ashwagandha; (ii) the first extended release tablet comprises a dose of about 78 mg L-theanine, about 11 mg saffron, and about 29 mg ashwagandha (iii) the second extended release tablet comprises a dose of about 78 mg L-theanine, about 18 mg saffron, and about 29 mg.

In some embodiments of the foregoing or related aspects, the matrix of the first extended release tablet comprises a rate controlling polymer. In some embodiments, the first extended release tablet comprises an amount of the rate controlling polymer, wherein the amount is about 5% to about 15% by weight of the tablet. In some embodiments, the coating of the first extended release tablet comprises a rate controlling polymer. In some embodiments, the first extended release tablet comprises an amount of the coating, wherein the amount is about 0.5% to about 5% by weight of the tablet. In some embodiments, the matrix of the second extended release tablet comprises a rate controlling polymer. In some embodiments, the second extended release tablet comprises an amount of the rate controlling polymer, wherein the amount is about 15% to about 25% by weight of the tablet. In some embodiments, the coating of the second extended release tablet further comprises a rate controlling polymer. In some embodiments, the coating of the second extended release tablet comprises an amount of the coating, wherein the amount is about 0.5% to about 3% by weight of the tablet. In some embodiments, the matrix of the immediate release tablet comprises a disintegrant. In some embodiments, the matrix of the immediate release tablet comprises an amount of the disintegrant, wherein the amount is about 0.5% to about 12% by weight of the tablet.

In some embodiments of the foregoing or related aspects, under gastrointestinal dissolution conditions (i) the immediate release tablet releases the dose in about 0.5 hours to about 2 hours; (ii) the first extended release tablet releases the dose in about 2 hours to about 5 hours; and/or (iii) the second extended release tablet releases the dose in about 6 hours to about 9 hours.

In some embodiments of the foregoing or related aspects, the rate controlling polymer comprises a cellulose polymer. In some embodiments, the cellulose polymer is selected from HPMC, HPC, MCC, and a combination thereof. In some embodiments, the rate controlling polymer comprises a mixture of low molecular weight (MW) HPMC and high MW HPMC, optionally a 1:1 mixture.

In some embodiments of the foregoing or related aspects, the capsule comprises a cylindrical shape having a circular cross-section transverse to a longest dimension, and wherein (i) the spherical tablets each comprise a diameter that is about 90% to about 99% of a diameter of the circular cross-section; (ii) the spherical tablets each comprise a diameter that is substantially the same; (iii) the spherical tablets comprise a combined volume that is about 90 to about 99% of the volume of the capsule; or (iv) a combination of (i)-(iii).

In some aspects, the disclosure provides a customized-release nutraceutical capsule, comprising three spherical tablets comprising an intermediate release tablet and an extended release tablet, wherein each spherical tablet comprises a coating and a matrix, wherein the matrix is substantially encapsulated by the coating, and wherein the matrix comprises a dose of a nutraceutical selected from L-theanine, saffron, ashwagandha, and a combination thereof, and wherein the three spherical tablets are enclosed in the capsule. In some embodiments, the three spherical tablets further comprise an immediate release tablet.

In some embodiments of the foregoing or related aspects, the immediate release tablet comprises (i) a dose of L-theanine, wherein the dose is about 60 mg to about 100 mg; (ii) a dose of saffron, wherein the dose is about 2 mg to about 10 mg; and/or (iii) a dose of ashwagandha, wherein the dose is about 20 mg to about 40 mg. In some embodiments, the intermediate release tablet comprises (i) a dose of L-theanine, wherein the dose is about 60 mg to about 100 mg; (ii) a dose of saffron, wherein the dose is about 8 mg to about 12 mg; and/or (iii) a dose of ashwagandha, wherein the dose is about 20 mg to about 40 mg. In some embodiments, the extended release tablet comprises (i) a dose of L-theanine, wherein the dose is about 60 mg to about 100 mg; (ii) a dose of saffron, wherein the dose is about 12 mg to about 20 mg; and/or (iii) a dose of ashwagandha, wherein the dose is about 20 mg to about 40 mg. In some embodiments, (i) the immediate release tablet comprises about 60 mg to about 100 mg L-theanine, about 2 mg to about 10 mg saffron, and about 20 mg to about 40 mg ashwagandha; (ii) the intermediate release tablet comprises a dose of about 60 mg to about 100 mg L-theanine, about 8 mg to about 12 mg saffron, and about 20 mg to about 40 mg ashwagandha; and (iii) the extended release tablet comprises a dose of about 60 mg to about 100 mg L-theanine, about 15 mg to about 20 mg saffron, and about 20 mg to about 40 mg ashwagandha. In some embodiments, (i) the immediate release tablet comprises about 78 mg L-theanine, about 5 mg saffron, and about 29 mg ashwagandha; (ii) the intermediate release tablet comprises a dose of about 78 mg L-theanine, about 11 mg saffron, and about 29 mg ashwagandha; and (iii) the extended release tablet comprises a dose of about 78 mg L-theanine, about 18 mg saffron, and about 29 mg.

In some embodiments of the foregoing or related aspects, the matrix of the intermediate release tablet comprises a rate controlling polymer. In some embodiments, the intermediate release tablet comprises an amount of the rate controlling polymer, wherein the amount is about 5% to about 15% by weight of the tablet. In some embodiments, the coating of the intermediate release tablet comprises a rate controlling polymer. In some embodiments, the intermediate release tablet comprises an amount of the coating, wherein the amount is about 0.5% to about 5% by weight of the tablet. In some embodiments, the matrix of the extended release tablet comprises a rate controlling polymer. In some embodiments, the extended release tablet comprises an amount of the rate controlling polymer, wherein the amount is about 15% to about 25% by weight of the tablet. In some embodiments, the coating of the extended release tablet further comprises a rate controlling polymer. In some embodiments, the coating of the extended release tablet comprises an amount of the coating, wherein the amount is about 0.5% to about 3% by weight of the tablet. In some embodiments, the matrix of the immediate release tablet comprises a disintegrant. In some embodiments, the matrix of the immediate release tablet comprises an amount of the disintegrant, wherein the amount is about 0.5% to about 12% by weight of the tablet.

In some embodiments of the foregoing or related aspects, under gastrointestinal dissolution conditions (i) the immediate release tablet releases the dose in about 0.5 hours to about 2 hours; (ii) the intermediate release tablet releases the dose in about 2 hours to about 5 hours; and/or (iii) the extended release tablet releases the dose in about 6 hours to about 9 hours.

In some aspects, the disclosure provides a customized-release nutraceutical capsule, comprising: three spherical tablets comprising at least one extended release tablet; wherein each spherical tablet comprises a coating and a matrix, wherein the matrix is substantially encapsulated by the coating, wherein the matrix comprises a dose of iron; and wherein the three spherical tablets are enclosed in the capsule.

In some embodiments of any of the foregoing or related aspects, the dose of iron is about 1 mg to about 50 mg. In some embodiments, the dose of iron is about 20 mg. In some embodiments, the iron is ferrous bisglycinate. In some embodiments, at least spherical one tablet further comprises (i) a dose of vitamin A, (ii) a dose of vitamin C, or (iii) both (i) and (ii). In some embodiments, at least spherical one tablet further comprises the dose of vitamin C. In some embodiments, the vitamin C is ascorbic acid. In some embodiments, the dose of vitamin C is about 1 mg to about 100 mg. In some embodiments, the dose of vitamin C is about 6 mg, about 7 mg, about 8 mg, about 9 mg, about 10 mg, about 11 mg, about 12 mg, about 13 mg, about 14 mg, or about 15 mg. In some embodiments, at least one spherical tablet further comprises the dose of vitamin A. In some embodiments, the vitamin A is a provitamin A carotenoid. In some embodiments, the provitamin A carotenoid is beta-carotene. In some embodiments, the dose of vitamin A is about 10 mcg retinal activity equivalents (RAE) to about 3000 mcg RAE. In some embodiments, the dose of vitamin A is about 10 mcg RAE to about 500 mcg RAE. In some embodiments, the dose of vitamin A is about 80 mcg RAE to about 90 mcg RAE. In some embodiments, the dose of vitamin A is about 200 mcg RAE to about 300 mcg RAE.

In some embodiments of any of the foregoing or related aspects, (i) at least one spherical tablet is an intermediate release tablet; and/or (ii) at least one spherical tablet is an immediate release tablet. In some embodiments of any of the foregoing or related aspects, the three spherical tablets comprise an extended release tablet, an intermediate release tablet, and an immediate release tablet. In some embodiments, the extended release tablet, the intermediate tablet, and the immediate release tablet each comprises a dose of iron of about 1-50 mg, a dose of vitamin C of about 1-100 mg, and a dose of vitamin A of about 10-500 mcg RAE. In some embodiments, the extended release tablet, the intermediate release tablet, and the immediate release tablet each comprises a dose of iron of about 20 mg, a dose of vitamin C of about 8 mg, and a dose of vitamin A of about 87 mcg RAE. In some embodiments, (i) the extended release tablet comprises a dose of iron of about 1-50 mg and a dose of vitamin A of about 200-500 meg RAE; and (ii) the intermediate release tablet and the immediate release tablet each comprises a dose of iron of about 1-50 mg and a dose of vitamin C of about 1-100 mg. In some embodiments, (i) the extended release tablet comprises a dose of iron of about 20 mg and a dose of vitamin A of about 260 meg RAE; and (ii) the intermediate release tablet and the immediate release tablet each comprises a dose of iron of about 20 mg and a dose of vitamin C of about 12 mg. In some embodiments, the extended release tablet does not contain vitamin C and the intermediate release tablet and the immediate release tablet do not contain vitamin A. In some embodiments, the extended release tablet, the intermediate release tablet, and the immediate release tablet each comprises a dose of iron of about 1-50 mg and a dose of vitamin C of about 1-100 mg. In some embodiments, the extended release tablet, the intermediate release tablet, and the immediate release tablet each comprises a dose of iron of about 20 mg and a dose of vitamin C of about 12 mg. In some embodiments, the extended release tablet, the intermediate release tablet, and the immediate release tablet do not contain vitamin A. In some embodiments, the matrix and/or the coating of the extended release tablet comprises a rate controlling polymer. In some embodiments, the matrix of the extended release tablet comprises an amount of the rate controlling polymer, wherein the amount is about 2% to about 25% by weight of the tablet. In some embodiments, the matrix and/or the coating of the intermediate release tablet comprises a rate controlling polymer. In some embodiments, the matrix of the intermediate release tablet comprises an amount of the rate controlling polymer, wherein the amount is about 1% to about 15% by weight of the tablet. In some embodiments, the rate controlling polymer comprises a cellulose polymer. In some embodiments, the cellulose polymer is selected from HPMC, HPC, MCC, and a combination thereof. In some embodiments, the matrix of the immediate release tablet comprises a disintegrant. In some embodiments, the immediate release tablet comprises an amount of the disintegrant, wherein the amount is about 0.1% to about 5% by weight of the tablet.

In some embodiments of any of the foregoing or related aspects, under gastrointestinal dissolution conditions, (i) the extended release tablet releases the dose of iron in about 4 hours to about 8 hours, or about 4, 5, 6, 7, or 8 hours, under gastrointestinal dissolution conditions; (ii) the intermediate tablet releases the dose of iron in about 2 hours to about 6 hours, or about 2, 3, 4, 5, or 6 hours, under gastrointestinal dissolution conditions; and/or (iii) the immediate release tablet releases the dose of iron in about 0.5 to about 2 hours under gastrointestinal dissolution conditions. In some embodiments, the three spherical tablets comprise a color that is substantially the same or different.

In some embodiments of any of the foregoing or related aspects, the three spherical tablets comprise the extended release tablet as a first extended release tablet, a second extended release tablet, and an immediate release tablet. In some embodiments, the first extended release tablet, the second extended release tablet, and the immediate release tablet each comprises a dose of iron of about 1-50 mg, a dose of vitamin C of about 1-100 mg, and a dose of vitamin A of about 10-500 mcg RAE. In some embodiments, the first extended release tablet, the second extended release tablet, and the immediate release tablet each comprises a dose of iron of about 20 mg, a dose of vitamin C of about 8 mg, and a dose of vitamin A of about 87 mcg RAE. In some embodiments, (i) the first extended release tablet comprises a dose of iron of about 1-50 mg and a dose of vitamin A of about 200-500 mcg RAE; and (ii) the second extended release tablet and the immediate release tablet each comprises a dose of iron of about 1-50 mg and a dose of vitamin C of about 1-100 mg. In some embodiments, (i) the first extended release tablet comprises a dose of iron of about 20 mg and a dose of vitamin A of about 260 mcg RAE; and (ii) the second extended release tablet and the immediate release tablet each comprises a dose of iron of about 20 mg and a dose of vitamin C of about 12 mg. In some embodiments, the first extended release tablet does not contain vitamin C and the second extended release tablet and the immediate release tablet do not contain vitamin A. In some embodiments, the first extended release tablet, the second extended release tablet, and the immediate release tablet each comprises a dose of iron of about 1-50 mg and a dose of vitamin C of about 1-100 mg. In some embodiments, the first extended release tablet, the second extended release tablet, and the immediate release tablet each comprises a dose of iron of about 20 mg and a dose of vitamin C of about 12 mg. In some embodiments, the first extended release tablet, the second extended release tablet, and the immediate release tablet do not contain vitamin A. In some embodiments, the matrix and/or the coating of the first extended release tablet comprises a rate controlling polymer. In some embodiments, the matrix and/or the coating of the second extended release tablet comprises a rate controlling polymer. In some embodiments, the matrix of the first extended release tablet comprises an amount of the rate controlling polymer, wherein the amount is about 2% to about 25% by weight of the tablet. In some embodiments, the matrix of the second extended release tablet comprises an amount of the rate controlling polymer, wherein the amount is about 1% to about 15% by weight of the tablet. In some embodiments, the rate controlling polymer comprises a cellulose polymer. In some embodiments, the cellulose polymer is selected from HPMC, HPC, MCC, and a combination thereof. In some embodiments, the matrix of the immediate release tablet comprises a disintegrant. In some embodiments, the immediate release tablet comprises an amount of the disintegrant, wherein the amount is about 0.1% to about 5% by weight of the tablet.

In some embodiments of any of the foregoing or related aspects, under gastrointestinal dissolution conditions, (i) the first extended release tablet releases the dose of iron in about 2 hours to about 8 hours (e.g., about 4 hours to about 8 hours, or about 4, 5, 6, 7, or 8 hours), under gastrointestinal dissolution conditions; (ii) the second extended release tablet releases the dose of iron in about 2 hours to about 8 hours (e.g., about 4 hours to about 6 hours, or about 2, 3, 4, 5, or 6 hours), under gastrointestinal dissolution conditions; and/or (iii) the immediate release tablet releases the dose of iron in about 0.5 to about 2 hours under gastrointestinal dissolution conditions. In some embodiments, the three spherical tablets comprise a color that is substantially the same or different.

In some embodiments of the foregoing or related aspects, the capsule is formulated for instant release. In some embodiments, the capsule comprises Hypromellose. In some embodiments, the capsule is formulated for delayed release. In some embodiments, the capsule comprises HPMC and gellan gum. In some embodiments, the capsule is standard size 0, standard size 00, elongated 0, or elongated 00. In some embodiments, the capsule is semi-opaque or opaque. In some embodiments, the capsule is transparent. In some embodiments, the capsule comprises a colorant. In some embodiments, the capsule further comprises an oil, wherein the spherical tablets are surrounded by the oil. In some embodiments, the capsule further comprises an powder, wherein the spherical tablets are surrounded by the powder. In some embodiments, the spherical tablets are a modified ball shape. In some embodiments, the spherical tables each have a diameter that is substantially the same. In some embodiments, the spherical tablets each have a diameter of about 6 mm, about 6.4 mm, about 6.5 mm, about 6.7 mm, about 6.9 mm, about 7 mm, or about 7.5 mm. In some embodiments, the capsule and/or a component thereof comprises a vegan ingredient. In some embodiments, the capsule and/or a component thereof lacks an animal-derived ingredient. In some embodiments, the capsule and the components thereof are vegetarian.

In some aspects, the disclosure provides a pharmaceutical composition comprising a capsule described herein, and a pharmaceutically acceptable carrier.

In some aspects, the disclosure provides a method for providing a health benefit to a subject, comprising administering to the subject a capsule described herein or a pharmaceutical composition described herein.

In some aspects, the disclosure provides a method for biomimicking a circadian rhythm, a physiological rhythm, an endogenous hormone release pattern, or a biomarker including an enzyme or a protein.

In some aspects, the disclosure provides a method for improving sleep in a subject, comprising administering to the subject the capsule described herein. In some embodiments, the administering results in an increased total duration of sleep, a reduced time to fall asleep, a reduced number of nocturnal wakings, reduced fatigue following a sleep period, an improved quality of sleep, and/or increased sleep efficiency.

In some aspects, the disclosure provides a method for reducing stress and/or improving mood in a subject, comprising administering to the subject a capsule described herein. In some embodiments, the administering results in normalization of a cortisol level, heart rate, and/or blood pressure. In some embodiments, the administering results in a reduced score on the perceived stress scale.

In some aspects, the disclosure provides a method for improving sleep of a subject during a nocturnal sleep period, comprising administering to the subject a capsule described herein (e.g., a capsule described herein comprising a dose of melatonin) about 0.5 hours to about 2 hours prior to the nocturnal sleep period.

In some embodiments of any of the foregoing or related aspects, the nocturnal sleep period comprises a duration of about 6 hours to about 10 hours. In some embodiments, the nocturnal sleep period comprises a duration of about 6 hours, about 7 hours, or about 8 hours.

In some embodiments of any of the foregoing or related aspects, the subject experiences a reduction in a period for sleep latency, wherein the period for sleep latency is reduced by at least about 10%, about 20%, about 30%, about 40%, about 50%, or about 60% as compared to a subject not administered the capsule, optionally wherein sleep latency is measured by a Pittsburgh Sleep Quality Index (PSQI) survey or a wearable sleep tracker. In some embodiments, the period for sleep latency is reduced by about 5 minutes to about 20 minutes as compared to a subject not administered the capsule.

In some embodiments of any of the foregoing or related aspects, the subject experiences an improved waking up fresh and rested score as compared to a subject not administered the capsule, optionally wherein the score is measured by a quality of life WHO-5 survey. In some embodiments, the subject experiences substantially no side effect as compared to a subject not administered the capsule, wherein the side effect is tiredness, grouchiness, and/or sleepiness upon waking from the nocturnal sleep period, and optionally wherein the side effect is measured using a Rest-Q survey.

In some embodiments of any of the foregoing or related aspects, the subject does not substantially experience a rebound effect during or following a first nocturnal sleep period upon discontinued administration of the capsule. In some embodiments, the rebound effect is selected from increased sleep onset latency, reduced total sleep time, increased number of nocturnal awakenings and a combination thereof, as compared to a baseline nocturnal sleep period, optionally wherein the rebound effect is measured by a wearable sleep tracker. In some embodiments, the rebound effect is worsening of well-being following the nocturnal sleep period as compared to a baseline nocturnal sleep period, optionally wherein well-being following the nocturnal sleep period is measured by REST-Q.

In some embodiments of any of the foregoing or related aspects, the subject experiences increased deep sleep during a sleep cycle occurring about 1.5 hours to about 3 hours from onset of the nocturnal sleep period as compared to a subject not administered the capsule, optionally wherein deep sleep is measured by a wearable sleep tracker. In some embodiments, the subject experiences increased light sleep during a sleep cycle occurring about 6 or more hours from onset of the nocturnal sleep period as compared to a subject not administered the capsule, optionally wherein light sleep is measured by a wearable sleep tracker.

In some aspects, the disclosure provides a method of increasing nocturnal melatonin levels in a subject, comprising administering to the subject a capsule described herein (e.g., a capsule described herein comprising a dose of melatonin) about 0.5 hours to about 2 hours prior to the nocturnal sleep period. In some embodiments, the melatonin level is measured in the blood of the subject. In some embodiments, the melatonin level is increased within about 15 minutes to about 60 minutes of administering the capsule to the subject. In some embodiments, the increased melatonin level is maintained for up to about 6 hours from the time of administering the capsule. In some embodiments, an area under the curve (AUC) for the melatonin level measured over the nocturnal sleep period is increased by about 2-fold to about 50-fold compared to an AUC for a melatonin level in a subject not administered the capsule. In some embodiments, a peak concentration for the melatonin level is increased by about 2-fold to about 20-fold as compared to a peak concentration for a melatonin level of a subject not administered the capsule. In some embodiments, a time to peak for the melatonin level occurs about 2 hours to about 3 hours from the time of administering the capsule. In some embodiments, the time to peak occurs about 2 hours to about 3 hours earlier than a time to peak for a melatonin level of a subject not administered the capsule.

In some embodiments of any of the foregoing or related aspects, the subject is administered the capsule about 10 minutes to about 120 minutes prior to the nocturnal sleep period. In some embodiments, the subject is administered the capsule about 10 minutes to about 120 minutes prior to the nocturnal sleep period.

In some embodiments of any of the foregoing or related aspects, the subject consumes a single capsule or more than one capsule prior to the nocturnal sleep period. In some embodiments, the subject is male or female. In some embodiments, the subject is human. In some embodiments, the subject is an adult human, optionally an adult human of about 18 years to about 35 years of age. In some embodiments, the subject has a prior history of poor sleep quality, difficulty falling asleep, and/or frequent nocturnal wakings.

In some aspects, the disclosure provides a method for increasing, maintaining, or normalizing an iron level in a subject, comprising administering to the subject a capsule described herein (e.g., a capsule described herein comprising a dose of iron). In some embodiments, the administering is oral. In some embodiments, the subject is human. In some embodiments, the subject is male or female. In some embodiments, the subject is female. In some embodiments, the femail is pregnant. In some embodiments, the female is not pregnant (i.e., non-natal). In some embodiments, the subject is administered the capsule in a dosing regimen comprising a single capsule every about 24 hours to about 72 hours. In some embodiments, the dosing regimen comprises a single capsule every about 48 hours. In some embodiments, the subject is administered the capsule in the morning. In some embodiments, the administering the capsule to the subject results in increased iron levels, increased energy level, increased iron absorption, and/or substantially no gastrointestinal discomfort.

In some aspects, the disclosure provides a kit comprising a container comprising at least one capsule described herein, and a package insert comprising instructions for administering the capsule to a subject to provide one or more health benefits.

In some aspects, the disclosure provides a kit comprising a container comprising at least one capsule described herein, and a package insert comprising instructions for administering the capsule to a subject to improve sleep. In some embodiments, the container comprises at least one capsule described herein comprising a dose of melatonin.

In some aspects, the disclosure provides a kit comprising a container comprising at least one capsule described herein, and a package insert comprising instructions for administering the capsule to a subject to reduce stress. In some embodiments, the container comprises at least one capsule described herein comprising a dose of L-theanine, a dose of saffron, and/or a dose of ashwagandha.

In some aspects, the disclosure provides a kit comprising a container comprising at least one capsule described herein, and a package insert comprising instructions for administering the capsule to a subject to improve or maintain an iron level in the subject. In some embodiments, the container comprises at least one capsule described herein comprising a dose of iron.

In some embodiments of the foregoing or related aspects, at least one capsule is shelf-stable. In some embodiments, the container comprises about 10 to about 100 capsules. In some embodiments, the container comprises an insert, wherein the insert comprises a botanical oil, a flavorant, or both.

In some embodiments of the foregoing or related aspects, the administering is oral. In some embodiments, the administering is once daily or more than once daily.

In some aspects, the disclosure provides a method for reducing a side effect associated with a nutraceutical in a subject, comprising administering to the subject a capsule comprising two or more spherical tablets enclosed in the capsule, each comprising an effective dose of the nutraceutical, wherein at least one of the two or more spherical tablets is an extended release tablet, wherein the extended release tablet comprises one or more rate controlling polymers in an amount sufficient to release the dose of the nutraceutical over an extended duration of time, and

wherein the release of the dose following the administering provides a bioavailability of the nutraceutical that results in a health benefit to the subject without substantially inducing the side effect.

In some aspects, the disclosure provides a method for customized dosing of a nutraceutical to a subject, comprising administering to the subject a capsule comprising two or more spherical tablets enclosed in the capsule, each comprising an effective dose of the nutraceutical, wherein at least one of the two or more spherical tablets is an extended release tablet, and wherein the extended release tablet comprises one or more rate controlling polymers in an amount sufficient to release the dose of the nutraceutical over an extended duration of time. In some embodiments, the capsule is formulated to provide a customized release of the nutraceutical. In some embodiments, wherein the release of the nutraceutical mimics an endogenous circadian rhythm. In some embodiments, the release of the nutraceutical mimics an endogenous physiological pattern. In some embodiments, the release of the nutraceutical promotes optimal uptake by an endogenous cellular transport mechanism. In some embodiments, the release of the nutraceutical mimics an endogenous release of a biomarker. In some embodiments, the biomarker is a protein, and enzyme, or a hormone. In some embodiments, the administering results in a health benefit to the subject. In some embodiments, the health benefit is selected from the group consisting of (i) improved sleep quality and/or duration; (ii) reduced stress; (iii) improved mood; (iv) reduced anxiety; (v) improved immune function; (vi) reduced nutritional deficiency; (vii) improved cognition; (viii) improved hair quality; (ix) improved heart health; (x) reduced inflammation; (xi) increased energy; and (xii) a combination of (i)-(xi).

### BRIEF DESCRIPTION OF THE FIGURES

FIG. 1A is an image of representative 3-tablet-in-1-capsules. Shown are capsules of size 0 or size 00 and filled with three cylindrical tablets or three modified ball tablets.

FIG. 1B is an image of representative 3-tablet-in-1-capsules. Shown is a capsule of size 00 filled with three cylindrical tablets in alignment (i.e., each with flat surface in-parallel) or three cylindrical tablets with one mis-aligned relative to the other two (i.e., one tablet stacked vertical relative to the others).

FIG. 2 is a graph showing the amount of active ingredient (melatonin) released over time under gastrointestinal dissolution conditions for a tablet having 1 mg of melatonin and 10% by weight of rate controlling polymer (formula A), a tablet having 1 mg of melatonin and 15% by weight of rate controlling polymer (formula B), a tablet having 1.25 mg of melatonin and 15% by weight of rate controlling polymer (formula C), a tablet having 1.25 mg of melatonin and 15% by weight of rate controlling polymer (formula D), and a 1 mg of melatonin, 17% by weight of rate controlling polymer, and a coating (formula E). Tablets according to formulas A-D did not have a coating. The formulations were tested as single tablets in a capsule.

FIGS. 3A-3C are graphs showing the amount of melatonin released over time under gastrointestinal dissolution conditions for a tablet containing respectively 1 mg, 1.25 mg, or 3 mg melatonin and different amounts of rate controlling polymer or coating as defined in Table 4.

FIG. 4 is an image of a representative 3-tablet-in-1-capsule of the disclosure for administering a total dose of about 5 mg melatonin. Shown is a capsule filled with tablet 1 for complete dissolution of 1 mg melatonin within about 1 hour (colored dark shade), tablet 2 for complete dissolution of 3 mg melatonin within about 6-8 hours (colored light shade), and tablet 3 for complete dissolution of 1 mg melatonin within about 3 hours (colored white).

FIGS. 5A-5C are graphs showing the amount of melatonin (mg) released over time under gastrointestinal dissolution conditions for the 3-tablet-in-1-capsule described in FIG. 4. Shown in FIG. 5A is the dissolution per tablet (i.e., tablet 1 (“tab 1”), tablet 2 (“tab 2”), and tablet 3 (“tab 3”)); in FIG. 5B is the dissolution for tablet 1 as compared to tablet 2 and 3 combined; and in FIG. 5C is the dissolution for the entire capsule.

FIG. 6A is a graph showing the amount of melatonin (mg) released over time under gastrointestinal dissolution conditions for different lots of tablet 2 as described in FIG. 4.

FIG. 6B is a graph showing the amount of melatonin (mg) released over time under gastrointestinal dissolution conditions for tablet 3 as described in FIG. 4 either without a capsule or as a single tablet in a capsule.

FIG. 7 is a graph showing the amount of melatonin (mg) released over time under gastrointestinal dissolution conditions for the capsule described in FIG. 4 as compared to two commercially-available instant release melatonin tablets.

FIG. 8 is an image of a representative 3-tablet-in-1-capsule of the disclosure containing nutraceuticals associated with stress reduction (L-theanine, saffron, and ashwagandha). Shown is a capsule filled with tablet 1 for complete dissolution of 66.67 mg L-theanine, 4 mg saffron, and 26.67 mg ashwagandha within about 1 hour (“immediate release”); tablet 2 for complete dissolution of 66.67 mg L-theanine, 9 mg saffron, and 26.67 mg ashwagandha within about 4 hours (“intermediate release”); and tablet 3 for complete dissolution of 66.67 mg L-theanine, 15 mg saffron, and 26.67 mg ashwagandha within about 8 hours (“extended release”).

FIGS. 9A-9C are graphs showing the amount of L-theanine released over time under gastrointestinal dissolution conditions for the capsule described in FIG. 8. Shown in FIG. 9A is the dissolution per tablet represented as % L-theanine released over time; in FIG. 9B is the dissolution per tablet represented as mg L-theanine released over time; and in FIG. 9C is the dissolution for the entire capsule.

FIG. 10 is a diagram of a clinical study used to investigate the pharmacokinetics and effect on sleep quality parameters of a 3-tablet-in-1-capsule dosage form containing melatonin, as described in FIG. 4 and Example 4. The study has two arms and uses a randomized, double-blind, placebo-controlled, crossover design, as described in Example 7.

FIG. 11 shows mean (left) and area under the curve (AUC) (right) (±SEM) serum melatonin concentration over time, measured by ELISA, during nocturnal sleep and following ingestion of placebo (open symbols) or the sleep supplement (closed symbols) one hour before lights out. Data was analyzed by mixed model 2-way ANOVA.

FIG. 12 is a graph showing the duration of deep sleep for individuals treated with placebo (white bars) or the sleep supplement (black bars) as described in Example 8. The duration of deep sleep is shown for the five categories of sleep indicated on the x-axis: 90 minute intervals from 0-1.5 hours after falling asleep, 1.5-3 hours after falling asleep, 3-4.5 hours after falling asleep, and 4.5-6 hours after fall asleep, or 6+ hours after falling sleep. The * indicates that, compared to placebo, the sleep supplement significantly improved nocturnal deep sleep 1.5-3 hours into sleep (paired t-test: p<0.05).

FIG. 13 is an image of a representative 3-tablet-in-1-capsule of the disclosure for supplementing dietary iron in which each tablet contains a dose of iron.

### DETAILED DESCRIPTION

The present disclosure provides dosage forms comprising an active ingredient in a formulation, wherein the formulation allows for customized release of the active ingredient designed to meet the demands of endogenous transport mechanisms, mimic endogenous circadian or other physiological rhythms, and/or to mimic release of endogenous biomarkers (e.g., release of endogenous enzymes, proteins, and hormones). The disclosure further provides methods for achieving a health benefit in a subject by administering a dosage form described herein to the subject.

Conventional solid dosage forms, e.g., compressed tablets, are composed of active ingredients dissolved or embedded within a matrix. Such dosage forms are typically characterized by a release profile in which the plasma level of the active ingredient increases rapidly to a high level following administration and then declines rapidly. This presents a challenge for obtaining therapeutic efficacy, which may require an extended plasma half-life of the active ingredient, and presents safety issues if the active ingredient is toxic at high concentrations. Accordingly, there is a need for solid dosage forms that provide a customized release of an active ingredient. An aspect of controlled drug delivery is the ability to manipulate the dosage form in order to establish the desired kinetics of drug release. In some embodiments, the active ingredient is formulated as a solid dosage form for sustained drug delivery, wherein the release of the active ingredient from the formulation is protracted over a period of time, e.g., hours. In some embodiments, the release of the active ingredient provides consistent levels of the active ingredient over a time period ranging from an hour to a day. In some embodiments, the release profile is characterized by the absence of an immediate release phase.

There are limited options for customizing the release profile of solid dosage forms to obtain prolonged delivery and maintenance of therapeutic plasma levels of an active ingredient for an extended duration. For example, pulsatile drug delivery systems offer control over active ingredient release, but require complex manufacturing processes and have limited drug loading capacity (see, e.g., Jain et al (2011) Biomatter 1:1, 57-65). As another example, layer-by-layer products, with each layer formulated to release an encapsulated active ingredient at a desired rate, provide extended and/or customized release profiles. Such products however are prone to batch-to-batch variability as the compression of one layer may impact the other layers and diminish the consistency of the release profile. A solid dosage form that provides consistent and inexpensive manufacture, a customizable release profile, high drug loading capacity, and capability to combine multiple active ingredients would offer advantages over conventional drug delivery systems, including increased patient compliance, improved pharmacological action, reduced side effects, and reduced dosing frequency.

Accordingly, the present disclosure is based, at least in part, on the development of a solid dosage form for administering a nutraceutical (e.g., 1, 2, 3, 4, or more nutraceuticals) in a customizable release profile without requiring a complicated manufacturing process. As described herein, the disclosure provides a solid dosage form comprising multiple tablets (e.g., 2, 3, 4, 5, or more tablets) within a single capsule. Each tablet comprises a composition formulated to provide a desired dissolution profile. Indeed, as described herein, tablet formulations were identified for altering the dissolution profile of a nutraceutical. For example, it was demonstrated that including a rate controlling polymer in the matrix of the tablet and/or an increased amount of coating resulted in a tablet having an extended release profile. Furthermore, it was demonstrated that increasing the amount of nutraceutical in the tablet provided a higher initial burst release of the nutraceutical.

An advantage of the solid dosage forms described herein is that the tablets present in the plurality contained in the single capsule are not required to have the same composition or the same release profile. Rather, in some embodiments, the multiple tablets comprise different compositions, wherein each tablet is formulated to have a unique dissolution profile relative to the other tablets in the capsule. For example, in some embodiments, the capsule comprises a tablet formulated for rapid dissolution and a tablet formulated for dissolution over an extended duration. As described herein, by combining a plurality of tablets characterized by distinct dissolution profiles into a single capsule, a solid dosage form is achieved having a customized dissolution profile that is based on the dissolution profile of the individual tablets in the capsule. For example, in some embodiments, modification of the amount of a rate-controlling polymer, the amount of coating, and/or the amount of an active ingredient present in the individual tablets achieves a solid dosage form having a customized release profile. In some embodiments, the customized release profile is selected from immediate release, intermediate release, extended release, delayed release and/or pulsatile release. Moreover, in some embodiments, the color of the individual tablets is altered such that each tablet of the plurality comprises a distinct color. This has the benefit of rendering the tablets readily distinguishable from one another, which provides improved quality control and efficiency during the manufacturing process. Furthermore, in some embodiments, the plurality of tablets of the solid dosage form comprise a spherical form and the capsule comprises a cylindrical form.

In some embodiments, each tablet of the plurality comprises a nutraceutical or a combination of nutraceuticals. In some embodiments, the nutraceutical is for improving sleep. In some embodiments, the nutraceutical is for reducing stress. In some embodiments the nutraceutical comprises iron for preventing or ameliorating an iron deficiency. In some embodiments, the nutraceutical is for supplementing dietary vitamins and/or minerals. In some embodiments, the disclosure further provides methods for providing a health benefit in a subject by administering to the subject a dosage form described herein. In some embodiments, the administering is oral.

### Definitions

As used herein, the indefinite articles “a” and “an” and the definite article “the” are intended to include both the singular and the plural, unless the context in which they are used clearly indicates otherwise. “At least one” and “one or more” are used interchangeably to mean that the article may include one or more than one of the listed elements.

Throughout this application, the term “about” is used to indicate that a value includes the inherent variation of error for the device or the method being employed to determine the value, or the variation that exists among the samples being measured. Unless otherwise stated or otherwise evident from the context, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents. Unless otherwise indicated, it is to be understood that all numbers expressing quantities, ratios, and numerical properties of ingredients, reaction conditions, and so forth, used in the specification and claims are contemplated to be able to be modified in all instances by the term “about”.

As used herein, the term “circadian rhythm” refers to a physiological process that alters a biological function in an oscillating manner that recurs daily. Non-limiting exemplary biological functions regulated by a circadian rhythm include the sleep-wake cycle, endocrine secretion (e.g., melatonin, cortisol, growth hormone, prolactin), hepatic metabolism, renal function, glucose homeostasis, cardiovascular function, and core body temperature.

As used herein, “controlled release” or “customized release” refers to release of an active ingredient from a solid dosage form in a controlled fashion following administration that achieves a desired pharmacokinetic profile.

As used herein, the term “solid dosage form” refers to a dosage form that is a solid at ambient conditions.

As used herein, the term “capsule” includes instant release capsules, sustained release capsules, coated instant release capsules, coated sustained release capsules, delayed release capsules and coated delayed release capsules.

As used herein, the term “tablet” generally refers to a tablet for oral ingestion, e.g., prepared by a single compression or by pre-compaction tapping followed by a final compression.

As used herein, the terms “active,” “active agent,” and “active ingredient” are used interchangeably and refer to a compound in a solid dosage form described herein intended to have a desired effect (e.g., providing a health benefit). The term encompasses any molecule, chemical, composition, drug, active ingredient, or biological agent for preventing, ameliorating, or treating a disease or disorder in an individual. Further, the term encompasses small molecules (e.g., compounds comprising a molecular weight less than about 1 kDa), peptides, proteins, nucleic acids, inorganic compounds or alloys of inorganic compounds, carbohydrates, lipids, and combinations thereof.

As used herein, the term “gastrointestinal dissolution conditions” as used in reference to release of an active ingredient from a solid dosage form described herein or a tablet thereof refers a dissolution test as defined by the USP (see, e.g., The United States Pharmacopcial Convention, 2011, world wide web: ftp.uspbpep.com/v29240/usp29nf24s0_c711.html). In some embodiments, the dissolution test comprises exposing the solid dosage form or tablet thereof to release medium (e.g., 900 mL 0.1 M HCl) in a USP Apparatus 1 (basket) or Apparatus 2 (paddle) at about 37° C. and about 100 rpm). In some embodiments, the release medium is partially or completely changed after a duration of more than 1 hour.

As used herein, the term “dissolution profile” is used interchangeably with “release profile” and refers to the rate of release of an active ingredient from a solid dosage form described herein or a component thereof (e.g., a capsule or a tablet thereof) under gastrointestinal dissolution conditions. In some embodiments, the dissolution profile is obtained by withdrawing samples from the apparatus comprising the solid dosage form or the component thereof exposed to gastrointestinal dissolution conditions and quantifying the active ingredient therein. Methods for quantifying an amount of an active ingredient are known in the art and include, e.g., HPLC. In some embodiments, the dissolution profile for the solid dosage form or the component thereof is the plot of the amount of active ingredient released over time following exposure to gastrointestinal dissolution conditions.

As used herein, the term “immediate release” as used in reference to a solid dosage form described herein or a component thereof (e.g., a capsule or a tablet thereof) refers to one comprising a formulation that dissolves without substantially prolonging release of an active ingredient contained therein (e.g., upon exposure to gastrointestinal dissolution conditions or following oral administration).

As used herein, the term “extended release” as used in reference to a solid dosage form described herein or a component thereof (e.g., a capsule or a tablet thereof) refers to one comprising a formulation that releases an active ingredient contained therein over an extended period of time (e.g., upon exposure to gastrointestinal dissolution conditions or following oral administration). In some embodiments, the extended period of time is at least about 1 hour to achieve 85% or higher release of the active ingredient (e.g., upon exposure to gastrointestinal dissolution conditions or following oral administration). In some embodiments, the extended period of time is at least about 2 hours to achieve 85% or higher release of the active ingredient (e.g., upon exposure to gastrointestinal dissolution conditions or following oral administration). In some embodiments, the extended period of time is at least about 4 hours to achieve 85% or higher release of the active ingredient (e.g., upon exposure to gastrointestinal dissolution conditions or following oral administration). In some embodiments, a solid dosage form described herein comprises at least two tablets formulated for extended release, wherein a first tablet of the at least two tablets comprises a faster release profile than the second tablet of the at least two tablets. In such embodiments, the first tablet is referred to as an “intermediate release tablet” and the second tablet is referred to as an “extended release tablet.” For example, in some embodiments, a solid dosage form described herein comprises at least two tablets formulated for extended release, wherein (i) a tablet of the at least two tablets releases at least about 85% of an active ingredient contained therein over about 2 hours to about 6 hours, and (ii) a tablet of the at least two tablets release at least about 85% of an active ingredient contained therein over about 6 hours to about 12 hours, wherein (i) and (ii) are measured upon exposure to gastrointestinal dissolution conditions or following oral administration. In some embodiments described herein, the tablet of (i) is referred to as an “intermediate release tablet” and the tablet of (ii) is referred to as an “extended release tablet.”

As used herein, the term “nutraceutical” refers to an active ingredient that is marketable as a dietary supplement, e.g., under the U.S. Federal Food, Drug, and Cosmetic Act (FDCA) (22 USC §§ 301 et seq) and Dietary Supplement Health and Education Act (DSHEA) of 1994, as well as products sold outside a specific regulatory regime having a medicinal or drug-like benefit. A dietary supplement refers to a product taken orally that contains a dietary ingredient intended to supplement diet. Dietary ingredients include, but are not limited to, vitamins, minerals, herbs or other botanical ingredients, amino acids, enzymes, and metabolites.

A “probiotic” refers to a living strain of bacteria that, when administered in adequate amounts, confers a health benefit to the host (see Hill et al (2014) Nat Rev Gastroenterol Hepatol 11:506).

A “prebiotic” refers to a non-digestible food ingredient that beneficially affects the host by selectively stimulating the growth and/or activity of one or a limited number of bacteria in the colon. (Gibson G R, et al, J Nutr. 1995 June; 125 (6): 1401-12).

A “postbiotic” refers to a functional bioactive compound generated during fermentation that may promote health (see Wegh, et al (2019) Int J Mol Sci 20:4673).

As used herein, the term “botanical” refers to a compound derived from or relating to plants. In some embodiments, the botanical is obtained from a plant.

As used herein, the term “extract” refers to extracted matter obtained from a starting material that is botanical in origin (see, e.g., USP General Chapter <565>Botanic Extracts United States Pharmacopcia).

As used herein, a “nootropic” refers to a compound associated with improved cognition, e.g., by altering cellular function and/or metabolism in the central nervous system. The term encompasses naturally-occurring molecules, semi-synthetic molecules (e.g., a naturally occurring molecule derivatized by a chemical reaction(s)), and synthetic molecules.

As used herein, a “non-stimulant nootropic” refers to a nootropic that when administered to a subject does not substantially induce a physiological function(s) associated with consumption of a central nervous system stimulant (e.g., increased heart rate, blood pressure, and/or breathing rate).

A “binder” refers to a substance added to a tableting mixture to improve cohesion and plasticity, which in turn enhances processability when formulated into a tablet.

A “lubricant,” “flow aid” or a “glidant” each refer to a substance added to a tableting mixture for the purpose of improving flow properties, reducing interparticle friction, and reducing sticking to machinery used in tablet production.

A “disintegrant” refers to a substance added to a tableting mixture for the purpose of facilitating disintegration and dissolution into smaller particles that dissolve more rapidly into the gastrointestinal fluid following enteral administration (see USP 41-NF 36 General Chapter <701>Disintegration).

A “colorant” refers to a substance added to a tableting mixture for the purpose of providing color.

As used herein, the term “bioavailability” refers to the fraction of a dose of an active ingredient described herein available at a site of action following administration (e.g., oral administration) to a subject. The term “site of action” refers to a bodily compartment and/or a tissue site comprising the biological target(s) of the active ingredient, wherein modulation of the biological target(s) is effective for achieving a desired therapeutic effect.

As used herein, the term “oral bioavailability” refers to the fraction of a dose of an active ingredient described herein administered to a subject orally that is available at the site of action following the administration.

As used herein, the term “Cmax” refers to the maximum observed plasma concentration of an active ingredient following administration to a subject.

As used herein, the term “Tmax” refers to the time point of maximum observed plasma concentration of an active ingredient following administration to a subject.

As used herein, the term “AUC” refers to the “area under the curve” of a plot of concentration of an active ingredient present in a tissue sample (e.g., a blood sample) obtained from a subject following administration of the active ingredient versus time. As is understood by the skilled artisan, AUC is determined using mathematical approaches that calculate the concentration from time interval zero to infinity (“AUC total”) or a predetermined time point (e.g., calculation of concentration over a time interval of zero to 7 days provides “AUC0-7”).

As used herein, the term “percent bioavailability” (also referred to as “F”) represents the fraction of the amount of an active ingredient measured in circulation when orally administered as compared to the amount of the active ingredient measured in circulation when administered systemically (e.g., by intravenous injection). The calculation of F is determined by measuring the AUC (e.g., total AUC) for the active ingredient when orally administered (the “test route”) compared to the AUC when administered systemically (“systemic route”). For example, in some embodiments, the following equation is used to calculate percent bioavailability: F (%)=AUCₜₒₜₐₗ (test route)/AUCₜₒₜₐₗ (systemic route). This is an important term that establishes the relative fraction of the active ingredient entering circulation via the test route compared to the maximum possible amount entering circulation via the systemic route.

As used herein, the term “peak concentration” refers to the highest concentration of an active ingredient measured in a subject following administration. In some embodiments, the concentration of the active ingredient is measured in blood (e.g., serum or plasma) obtained from the subject at regular time intervals following the administration, wherein the highest concentration measured is the peak concentration.

As used herein, the term “time to peak” or “time to peak concentration” refers to the time at which peak concentration of an active ingredient is measured in a subject following administration. In some embodiments, the concentration of the active ingredient is measured in blood (e.g., serum or plasma) obtained from the subject at regular time intervals following the administration, wherein the time at which the highest concentration measured is the time to peak concentration.

As used herein, the term “subject,” “patient,” “individual,” are used interchangeably and refer to a mammal to having a condition or disorder to be prevented, ameliorated, or treated by ingesting a solid dosage form described herein. In some embodiments, the subject is a human. In some embodiments, the subject is an adult human.

As used herein, the term “disorder” refers to a state of health in a subject in which the subject maintains homeostasis, but that is less favorable than in the absence of the disorder.

As used herein, the term “disease” refers to a state of health in a subject in which the subject cannot maintain homeostasis, and wherein if the disease is not ameliorated, the subject's health will deteriorate.

As used herein, the term “rate controlling polymer” in reference to a solid dosage form described herein (e.g., a solid dosage form comprising a plurality of tablets in a capsule, the tablets of the plurality comprising an active ingredient) is a polymer that upon hydration of the solid dosage form will swell and form an expanded network through which the diffusion path of an active ingredient contained therein becomes longer. Such polymers may also reduce the rate of water uptake by the formulation and thus enable a more prolonged dissolution and release of the active ingredient. In some embodiments, the rate controlling polymer is capable of binding molecularly to the active ingredient via physical (and therefore reversible) interactions, thus increasing the effective molecular weight of the active ingredient and thus further modifying its permeation (diffusion) characteristics through the epithelial and basal membranes of the sublingual mucosa. Such binding is reversible in nature and does not involve any chemical modifications of the active ingredient. In some embodiments, the rate controlling polymer upon hydration forms a three-dimensional structure that entraps the active ingredient and prolongs its release from the dosage form.

As used herein, the term “therapeutically effective” amount of an active refers to an amount sufficient to produce a therapeutically (e.g., clinically) desirable result.

As used herein, the term “retinol activity equivalent” or “RAE” refers to a unit of measurement for vitamin A. In some embodiments, 1 microgram (mcg) RAE is equivalent to 1 mcg retinol. In some embodiments, 1 mcg RAE is equivalent to about 12-16 mcg of a pro-vitamin A carotenoid other than beta-carotene. In some embodiments, 1 mcg RAE is equivalent to about 6 mcg beta-carotene.

As used herein, the term “nocturnal sleep period” refers to a sleep period occurring at night. In some embodiments, the sleep period is characterized by a state of altered consciousness, e.g., including a decline in consciousness, a decrease motor output, and/or minimal responsiveness to the environment.

As used herein, the term “baseline nocturnal sleep period” refers to a nocturnal sleep period characterized by an average sleep parameter(s) (e.g., sleep efficiency, sleep quality, sleep latency, and/or sleep duration) measured over at least 3 nocturnal sleep periods (e.g., at least 3 consecutive nocturnal sleep periods) prior to administration of a solid dosage form described herein. For example, in some embodiments, the baseline sleep period is characterized by the average sleep parameter(s) measured over about 7 consecutive nocturnal sleep periods prior to administration of a solid dosage form described herein.

As used herein, the term “sleep latency” refers to the amount of time from “lights out,” or bedtime, to falling asleep.

As used herein, the term “sleep efficiency” refers to the proportion of time during a sleep episode that is actually spend sleeping. It is calculated by dividing total sleep time by total time in bed.

As used herein, the terms “deep sleep” or “slow-wave sleep” each refer to the last stage of non-rapid eye movement (NREM) sleep. As appreciate by the skilled artisan, sleep is characterized by a cycle having two phases: rapid eye movement (REM) sleep and NREM sleep. On average in adult humans, the cycle has a duration of about 80 to about 100 minutes and occurs about 4 to about 6 times per nocturnal sleep period (see, e.g., word wide web: nhlbi.nih.gov/health/sleep/stages-of-sleep #: ˜: text=When % 20you %20sleep %2C %20you %20cycle,wake %20up %20briefly %20between % 20cycles). NREM sleep is characterized by three sleep stages. The first stage is the transition between wakefulness and sleep; the second stage is the state of being asleep; and the third stage is deep sleep.

As used herein, the term “light sleep” refers to the first stage, or the first and second stage, of NREM sleep.

As used herein, the term “sleep duration” refers to the quantity of time that a person sleeps. In some embodiments, the sleep duration is measured per 24-hour day. In some embodiments, the sleep duration is measured per sleep period.

As used herein, the term “sleep tracker” refers to a device that tracks a person's sleep. A “wearable sleep tracker” refers to a device that is worn on the body to collect sleep biometrics (e.g., sleep quality and duration). Exemplary wearable sleep trackers include Fitbit, OuraRing, Whoop, and AppleWatch.

### Multi-Tablet-in-Capsule Solid Dosage Form

Provided herein are solid dosage forms comprising a plurality of tablets contained in a single capsule. In some embodiments, the tablet comprises a coating. For example, in some embodiments, the tablet comprises a coating that dissolves upon ingestion or upon contact with diluent. In some embodiments, the coating is characterized by immediate or substantially immediate dissolution upon the ingestion or the contact. In some embodiments, the coating is characterized by delayed dissolution following the ingestion or the contact. In some embodiments, the plurality of tablets are surrounded by air enclosed in the capsule. In some embodiments, the capsule comprises the plurality of tablets and an oil. In some embodiments, the plurality of tablets are surrounded by the oil. In some embodiments, the oil comprises an active ingredient described herein. In some embodiments, the capsule comprises the plurality of tablets and a powder. In some embodiments, the plurality of tablets are surrounded by the powder. In some embodiments, both the plurality of tablets and the powder are encapsulated by the capsule. In some embodiments, the powder comprises an active ingredient described herein.

In some embodiments, each tablet of the plurality comprises an active ingredient (e.g., 1, 2, 3, 4 or more active ingredients). In some embodiments, the active ingredient is a small molecule. In some embodiments, the active ingredient is an inorganic compound or alloy thereof. In some embodiments, the active ingredient is a carbohydrate. In some embodiments, the active ingredient is a lipid or an oil. In some embodiments, the active ingredient is a peptide or protein. In some embodiments, the active is a naturally occurring substance or a derivative thereof. In some embodiments, the active is a synthetic (i.e., chemically synthesized) substance.

In some embodiments, each tablet of the plurality comprises the same active ingredient(s). In some embodiments, the active ingredient(s) are present in substantially the same dose in each tablet of the plurality. In some embodiments, each tablet of the plurality comprises a different active ingredient. In some embodiments, each tablet of the plurality comprises the same active ingredient in a dose, wherein the dose is different in each tablet of the plurality.

In some embodiments, the solid dosage form comprises at least two tablets contained in a single capsule. In some embodiments, the solid dosage form comprises at least three tablets contained in a single capsule. In some embodiments, the solid dosage form comprises at least four tablets contained in a single capsule. In some embodiments, the solid dosage form comprises at least five tablets contained in a single capsule. In some embodiments, the single capsule comprises the tablets surrounded by air. In some embodiments, the single capsule comprises the tablets surrounded by an oil. In some embodiments, the oil comprises an active ingredient. In some embodiments, the active ingredient contained in the oil is the same or different than the active ingredient contained in the tablets. In some embodiments, the single capsule comprises the tablets surrounded by a powder. In some embodiments, the powder comprises an active ingredient. In some embodiments, the active ingredient contained in the powder is the same or different than the active ingredient contained in the tablets.

In some embodiments, at least one tablet of the plurality is formulated for immediate release, e.g., under gastrointestinal dissolution conditions. In some embodiments, at least one tablet of the plurality is formulated for extended release, e.g., under gastrointestinal dissolution conditions. In some embodiments, at least one tablet of the plurality is formulated for immediate release and at least one tablet of the plurality is formulated for extended release, e.g., under gastrointestinal dissolution conditions. In some embodiments, at least two tablets of the plurality are formulated for extended release, e.g., under gastrointestinal dissolution conditions. In some embodiments, a tablet of the plurality is formulated for immediate release and at least two tablets of the plurality are formulated for extended release, e.g., under gastrointestinal dissolution conditions. In some embodiments, the at least two tablets formulated for extended release have a substantially similar time to dissolution (e.g., a substantially similar time to achieve at least about 85% dissolution of an active ingredient under gastrointestinal dissolution conditions). In some embodiments, the at least two tablets formulated for extended release have a different time to dissolution (e.g., a substantially different time to achieve at least about 85% dissolution of an active ingredient under gastrointestinal dissolution conditions). For example, in some embodiments, the at least two tablets formulated for extended release comprise a tablet comprising an intermediate release profile and a tablet comprising an extended release profile, wherein the tablet comprising the intermediate release profile provides a more rapid release of the active ingredient contained therein as compared to the tablet comprising the extended release profile, while still providing extended release of the active ingredient (e.g., as measured under gastrointestinal dissolution conditions).

In some embodiments, the solid dosage form or a component thereof (e.g., a capsule or a tablet thereof) formulated for immediate release is characterized by release of at least about 85% of an active ingredient contained therein in less than about 60 minutes under gastrointestinal dissolution conditions. In some embodiments, an immediate release solid dosage form or a component thereof (e.g., a capsule or a tablet thereof) undergoes rapid dissolution following oral administration to release the active ingredient contained therein for gastrointestinal absorption. In some embodiments, the solid dosage form or a component thereof (e.g., a capsule or a tablet thereof) formulated for immediate release is characterized by at least about 70%, about 75%, about 80%, or higher release of an active ingredient contained therein within about 1 hour following oral administration.

In some embodiments, a capsule and/or a tablet of a solid dosage form described herein is formulated to have extended release following oral administration. In some embodiments, the solid dosage form or a component thereof (e.g., a capsule or a tablet thereof) formulated for extended release is characterized by release of an active ingredient contained therein at a predetermined rate following oral administration. In some embodiments, the solid dosage form or a component thereof (e.g., a capsule or a tablet thereof) formulated for extended release is characterized by release of an active ingredient contained therein at a predetermined location in the gastrointestinal tract following oral administration. In some embodiments, the solid dosage form or a component thereof (e.g., a capsule or a tablet thereof) formulated for extended release is characterized by release of at least about 85% of an active ingredient contained therein in greater than about 60 minutes under gastrointestinal dissolution conditions. In some embodiments, the solid dosage form or a component thereof (e.g., a capsule or a tablet thereof) formulated for extended release is characterized by release of at least about 85% of an active ingredient contained therein in about 2 hours to about 4 hours, about 2 hours to about 6 hours, about 4 hours to about 8 hours, about 6 hours to about 8 hours, or about 6 hours to about 10 hours. In some embodiments, the extended release is pulsatile.

In some embodiments, at least one tablet of the plurality comprises a spherical form. In some embodiments, each tablet of the plurality comprises a spherical form. In some embodiments, at least one tablet of the plurality comprises a spherical form and the single capsule comprises a cylindrical form. In some embodiments, each tablet of the plurality comprises a spherical form and the single capsule comprises a cylindrical form.

In some embodiments, the plurality of tablets fill a substantial majority of the volume of the capsule, i.e., wherein the void volume of the capsule has less than about 10%, about 5%, about 3%, or about 1% of the total capsule volume. In some embodiments, each tablet of the plurality comprises a diameter, wherein the diameter is at least about 90% to about 99% of a diameter of a circular cross-section transverse to the longest dimension of the capsule. In some embodiments, each tablet of the plurality comprises a diameter of about 5 mm to about 15 mm, wherein the diameter is at least about 90% to about 99% of a diameter of a circular cross-section transverse to the longest dimension of the capsule. In some embodiments, each tablet of the plurality comprises a diameter of about 6 mm to about 8 mm, wherein the diameter is at least about 90% to about 99% of a diameter of a circular cross-section transverse to the longest dimension of the capsule. In some embodiments, each tablet comprises a spherical form having a diameter of about 6 mm to about 8 mm, wherein the diameter is at least about 90% to about 99% of a diameter of a circular cross-section transverse to the longest dimension of the capsule.

In some embodiments, the tablets of the plurality each comprise a diameter that is substantially similar. In some embodiments, the tablets of the plurality each comprises a spherical form having a diameter that is substantially similar.

In some embodiments, the tablets of the plurality comprise a combined volume, wherein the combined volume is at least about 90% to about 99% of the volume of the capsule. In some embodiments, the tablets of the plurality comprise a spherical form, wherein the combined volume of the spherical forms is at least about 90% to about 99% of the volume of the capsule.

### Tablets

The solid dosage forms of the disclosure comprise a plurality of tablets encapsulated in a single capsule. In some embodiments, the tablet comprises a matrix and a coating. In some embodiments, the coating surrounds the matrix. In some embodiments, the tablet comprises a single active ingredient, e.g., in the matrix. In some embodiments, the tablet comprises more than one active ingredient, e.g., in the matrix. In some embodiments, the tablets in the plurality comprise the same ingredients (e.g., the same active ingredients and excipients). In some embodiments, the tablets in the plurality comprise different ingredients (e.g., different active ingredients and/or excipients).

In some embodiments, the matrix comprises an active ingredient (e.g., 1, 2, 3, 4, or more active ingredients) and an excipient (e.g., 1, 2, 3, 4, or more excipients). In some embodiments, the excipient is a rate controlling polymer. In some embodiments, the excipient is a diluent. In some embodiments, the excipient is a binder. In some embodiments, the excipient is a lubricant. In some embodiments, the excipient is a glidant. In some embodiments, the excipient is a colorant.

In some embodiments, the matrix comprises an active ingredient (e.g., 1, 2, 3, 4, or more active ingredients) and an excipient (e.g., 1, 2, 3, 4, or more excipients), wherein the excipient is selected from a diluent, a binder, a lubricant, a flow aid, a colorant, a rate-controlling polymer, a lubricant, a disintegrant, and a combination thereof.

In some embodiments, the tablet is formulated for immediate release. In some embodiments, the tablet is formulated for immediate release under gastrointestinal dissolution conditions. In some embodiments, the tablet dissolves in about 10 to about 60 minutes under gastrointestinal dissolution conditions. In some embodiments, the tablet dissolves in about 10 minutes, about 20 minutes, about 30 minutes, about 40 minutes, about 50 minutes, or about 60 minutes under gastrointestinal dissolution conditions.

In some embodiments, a tablet formulated for immediate release comprises a matrix and a coating, wherein the matrix comprises an active ingredient (e.g., 1, 2, 3, 4, or more active ingredients) and a disintegrant. In some embodiments, the disintegrant comprises psyllium husk, oat fiber, or a combination thereof. In some embodiments, the matrix of the tablet formulated for immediate release comprises an active ingredient (e.g., 1, 2, 3, 4, or more active ingredients), a disintegrant, a lubricant, a diluent, and a glidant. In some embodiments, the matrix of the tablet formulated for immediate release comprises an active ingredient (e.g., 1, 2, 3, 4, or more active ingredients), a disintegrant, a lubricant, a diluent, a glidant, and a colorant. In some embodiments, the coating comprises a rate controlling polymer.

In some embodiments, the tablet formulated for immediate release comprises a coating in an amount, wherein the amount is about 0.5% to about 10% by weight of the tablet. In some embodiments, the amount is about 5% to about 10% by weight of the tablet. In some embodiments, the amount is about 1% to about 5% by weight of the tablet.

In some embodiments, the coating of the tablet formulated for immediate release comprises a rate controlling polymer (e.g., 1, 2, 3, 4, or more rate controlling polymers). In some embodiments, the coating comprises a cellulose derivative. In some embodiments, the coating comprises hydroxypropylmethylcellulose (HPMC), hydroxypropyl cellulose (HPC), microcrystalline cellulose (MCC), and a combination thereof.

In some embodiments, the tablet formulated for immediate release comprises an active ingredient in an amount, wherein the amount is at least about 0.05% to about 80% by weight of the tablet. In some embodiments, the amount is at least about 5% to about 60% by weight of the tablet. In some embodiments, the amount is at least about 10% to about 50% by weight of the tablet. In some embodiments, the amount is at least about 20% to about 40% by weight of the tablet. In some embodiments, the amount is at least about 5% to about 20% by weight of the tablet.

In some embodiments, the tablet is formulated for extended release. In some embodiments, a tablet formulated for extended release comprises a matrix and a coating, wherein the matrix comprises an active ingredient (e.g., 1, 2, 3, 4, or more active ingredients) and a rate controlling polymer (e.g., 1, 2, 3, 4, or more rate controlling polymers). In some embodiments, the rate controlling polymer is a cellulose derivative. In some embodiments, the cellulose derivative is selected from hydroxypropylmethylcellulose (HPMC), hydroxypropyl cellulose (HPC), microcrystalline cellulose (MCC), and a combination thereof. In some embodiments, the matrix of the tablet formulated for extended release comprises the rate controlling polymer in an amount, wherein the amount is about 1% to about 30% by weight of the tablet. In some embodiments, the matrix of the tablet formulated for extended release comprises the rate controlling polymer in an amount, wherein the amount is about 1% to about 15% by weight of the tablet. In some embodiments, the matrix of the tablet formulated for extended release comprises the rate controlling polymer in an amount, wherein the amount is about 5% to about 15% by weight of the tablet. In some embodiments, the matrix of the tablet formulated for extended release comprises the rate controlling polymer in an amount, wherein the amount is about 2% to about 25% by weight of the tablet. In some embodiments, the matrix of the tablet formulated for extended release comprises the rate controlling polymer in an amount, wherein the amount is about 15% to about 25% by weight of the tablet. In some embodiments, the amount of rate controlling polymer present in the matrix of the tablet is altered (e.g., increased or decreased) in order to customize the dissolution time. In some embodiments, the amount of rate controlling polymer present in the matrix of the tablet is increased in order to increase the dissolution time. In some embodiments, the amount of rate controlling polymer present in the matrix of the tablet is decreased in order to decrease the dissolution time.

In some embodiments, the matrix of the tablet formulated for extended release comprises an active ingredient (e.g., 1, 2, 3, 4, or more active ingredients), a rate controlling polymer (e.g., 1, 2, 3, 4, or more rate controlling polymers), diluent, a lubricant, and a glidant. In some embodiments, the matrix of the tablet formulated for extended release comprises an active ingredient (e.g., 1, 2, 3, 4, or more active ingredients), a rate controlling polymer (e.g., 1, 2, 3, 4, or more rate controlling polymers), diluent, a lubricant, a glidant, and a colorant.

In some embodiments, the tablet formulated for extended release comprises a coating in an amount, wherein the amount is about 0.5% to about 10% by weight of the tablet. In some embodiments, the amount is about 5% to about 10% by weight of the tablet. In some embodiments, the amount is about 1% to about 5% by weight of the tablet. In some embodiments, the coating of the tablet formulated for extended release comprises a rate controlling polymer (e.g., 1, 2, 3, 4, or more rate controlling polymers). In some embodiments, the coating comprises a cellulose derivative. In some embodiments, the coating comprises hydroxypropylmethylcellulose (HPMC), hydroxypropyl cellulose (HPC), microcrystalline cellulose (MCC), and a combination thereof. In some embodiments, the coating comprises a flow agent. In some embodiments, the flow agent is an alginate salt. In some embodiments, the flow agent is an oil (e.g., sunflower oil).

In some embodiments, the tablet formulated for extended release comprises an active ingredient in an amount, wherein the amount is at least about 0.05% to about 80% by weight of the tablet. In some embodiments, the amount is at least about 5% to about 60% by weight of the tablet. In some embodiments, the amount is at least about 10% to about 50% by weight of the tablet. In some embodiments, the amount is at least about 20% to about 40% by weight of the tablet. In some embodiments, the amount is at least about 5% to about 20% by weight of the tablet.

In some embodiments, the tablet has a shape according to any tablet design known in the art. In some embodiments, the tablet has a shape selected from spherical, square, rectangle, capsule, almond, pentagon, oval, lozenge, diamond, and core rod. In some embodiments, the tablet has a profile that is convex. In some embodiments, the profile is shallow, standard, deep, compound cup, convex bevel edge, flat face radius edge, flat face bevel edge, or modified ball. In some embodiments, the tablet comprises a spherical shape. In some embodiments, the spherical shape comprises a modified ball profile.

In some embodiments, the tablet comprises an average longest diameter of about 0.5 mm to about 15 mm, about 0.5 mm to about 10 mm, about 0.5 mm to about 8 mm, about 0.5 mm to about 6 mm, about 2 mm to about 15 mm, about 2 mm to about 10 mm, about 2 mm to about 8 mm, about 2 mm to about 6 mm, about 4 mm to about 15 mm, about 4 mm to about 10 mm, about 4 mm to about 8 mm, about 4 mm to about 6 mm, about 6 mm to about 15 mm, about 6 mm to about 10 mm, or about 6 mm to about 8 mm.

In some embodiments, the tablet has a spherical shape (e.g., a modified ball shape), wherein the spherical shape has a diameter of about 0.5 mm to about 15 mm, about 0.5 mm to about 10 mm, about 0.5 mm to about 8 mm, about 0.5 mm to about 6 mm, about 2 mm to about 15 mm, about 2 mm to about 10 mm, about 2 mm to about 8 mm, about 2 mm to about 6 mm, about 4 mm to about 15 mm, about 4 mm to about 10 mm, about 4 mm to about 8 mm, about 4 mm to about 6 mm, about 6 mm to about 15 mm, about 6 mm to about 10 mm, or about 6 mm to about 8 mm. In some embodiments, the spherical shape has a diameter of about 6 mm, about 7 mm, about 8 mm, about 9 mm, or about 10 mm.

In some embodiments, the tablet is formulated for extended release under gastrointestinal dissolution conditions. In some embodiments, the tablet dissolves in about 1 hour to about 12 hours under gastrointestinal dissolution conditions. In some embodiments, the tablet dissolves in about 2 hours, about 3 hours, about 4 hours, about 5 hours, about 6 hours, about 7 hours, about 8 hours, about 9 hours, about 10 hours, about 11 hours, or about 12 hours under gastrointestinal dissolution conditions. In some embodiments, the tablet dissolves in about 2 hours to about 3 hours, about 2 hours to about 4 hours, about 3 hours to about 5 hours, about 3 hours to about 6 hours, about 4 hours to about 7 hours, about 4 hours to about 8 hours, about 6 hours to about 9 hours, or about 8 hours to about 10 hours, under gastrointestinal dissolution conditions.

In some embodiments, the tablet is substantially free of certain excipients. As used herein, the term “substantially free” when used in relation to a given component of a solid dosage form described herein refers to a dosage form to which essentially none of said component has been added, such that the dosage form comprises no more than about 0.001 wt %, about 0.0001 wt %, or about 0.00001 wt % of said component.

In some embodiments, the tablet is substantially free of titanium (such as titanium dioxide), polymers synthesized from petroleum or petroleum products, polymers such as methacrylic ester or acrylic ester polymers, polyethylene oxides, polyvinylpyrrolidine, polyethylene glycols, talc, disaccharides (such as sucrose), lubricants (such as magnesium stearate or stearic acid), disintegrants such as croscarmellose sodium, lac resin (shellac), or any combinations thereof. In some embodiments, a tablet described herein comprises less than 0.1 wt %, less than 0.01 wt %, less than 0.001 wt %, or less than 0.0001 wt % of titanium (such as titanium dioxide), polymers synthesized from petroleum or petroleum products, polyvinylpyrrolidine, polyethylene glycols, talc, disaccharides (such as sucrose), or lac resin (shellac), or any combinations thereof.

In some embodiments, the tablet comprise a natural colorant. In some embodiments, the tablet is substantially free of synthetic colorants. For example, the coating of a tablet described herein is substantially free of FD&C Blue No. 1, FD&C Blue No. 2, FD&C Green No. 3, FD&C Yellow No. 5 (tartrazine), FD&C Yellow No. 6, or FD&C Red No. 3, FD&C Red No. 40, or the lake pigments of any of these, or any combinations thereof. In some embodiments, a tablet of the disclosure comprises less than 0.1 wt %, less than 0.01 wt %, less than 0.001 wt %, less than 0.0001 wt %, or less than 0.00001 wt % of FD&C Blue No. 1, FD&C Blue No. 2, FD&C Green No. 3, FD&C Yellow No. 5 (tartrazine), FD&C Yellow No. 6, FD&C Red No. 3, or FD&C Red No. 40, or the lake pigments of any of these, or any combinations thereof.

### Nutraceutical Active Ingredients

In some embodiments, the active ingredient is a nutraceutical. In some embodiments, the nutraceutical comprises a vitamin, a stimulant, a mineral, a plant extract, a prebiotic, a probiotic, a postbiotic, a botanical extract, a botanical oil, a synthetic active, a botanical ingredient, a plant-based ingredient, an amino acid, a metabolite, an enzyme, a nootropic, and a combination thereof.

In some embodiments, the nutraceutical comprises a single active ingredient or multiple active ingredients that function together to provide the desired benefit. Nutraceuticals have many health benefits, such as providing adequate amounts of essential nutrients, preventing chronic health conditions (e.g., cardiovascular disease, stroke, type 2 diabetes), or decreasing the risk of birth defects when consumed by pregnant women.

### (1) Minerals, Vitamins, and Probiotics

In some embodiments, the nutraceutical comprises a vitamin or dietary mineral. In some embodiments, the nutraceutical is selected from the group consisting of B vitamins, boron, magnesium, zinc, probiotics, choline, iodine, chromium, selenium, Vitamin A, Vitamin C, and iron. In some embodiments, the nutraceutical is selected from the group consisting of B vitamins, boron, magnesium, calcium, Vitamin A, and iron. In some embodiments, the nutraceutical comprises iron.

In some embodiments, the nutraceutical comprises a B vitamin. In some embodiments, the nutraceutical comprises a B vitamin selected from vitamin B1, vitamin B2, vitamin B3, vitamin B5, vitamin B7 (biotin), vitamin B9 (folate), vitamin B12, or a combination thereof. In some embodiments, the vitamin B3 is nicotinamide riboside. In some embodiments, the vitamin B3 is selected from pyridoxal-5′-phosphate, a pyridoxal-5′-phosphate salt, pyridoxine hydrochloride, or any combinations thereof. In some embodiments, the nutraceutical comprises vitamin B12. In some embodiments, the vitamin B12 is methylcobalamin. In some embodiments, the nutraceutical comprises folate. In some embodiments, the folate is a (6S)-5-methyltetrahydrofolate (5-MTHF) salt. In some embodiments, the nutraceutical comprises 5-MTHF glucosamine salt. In some embodiments, the folate is not folic acid. In some embodiments, the nutraceutical comprises vitamin B1. In some embodiments, the nutraceutical comprises vitamin B6. In some embodiments, the nutraceutical comprises vitamin B3. In some embodiments, the nutraceutical comprises vitamin B12 and folate. In some embodiments, the nutraceutical comprises vitamin B1, vitamin B6, and vitamin B3.

In some embodiments, the nutraceutical comprises boron. The boron may be present in any suitable form, for example as a boron compound. In some embodiments, the nutraceutical comprises a carbohydrate-boron complex. This may include complexes of boron with a sugar and/or sugar alcohol molecules. The sugar molecule may be a monosaccharide, disaccharide, trisaccharide, oligosaccharide, or polysaccharide, and the sugar alcohol may be any alcohols of any of those. Suitable sugars and sugar alcohols may include those with three to six carbons, for example pentoses or hexoses. The carbohydrate-boron complex may include complexes between boron and fructose, glucose, mannose, sorbose, sorbulose, sorbitol, xylose, xylulose, or xylitol. For example, the boron carbohydrate-boron complex may be fructoborate, or borogluconate. In some embodiments, the boron is in the form of a salt of a carbohydrate-boron complex, for example a calcium, magnesium, manganese, iron, copper, zinc, chromium, or vanadium salt. In some embodiments, the boron is in the form of a fructoborate salt. In some embodiments, the nutraceutical comprises calcium fructoborate. In other embodiments, the boron is in the form of a borogluconate salt, such as calcium borogluconate. In some embodiments, the boron is not boric acid, sodium borate, or boric anhydride.

In some embodiments, the nutraceutical comprises magnesium. The magnesium may be present in any suitable form, for example as a magnesium compound. In some embodiments, the magnesium is in the form of a magnesium salt. Magnesium salts may include magnesium ascorbate, magnesium aspartate, magnesium citrate, magnesium gluconate, magnesium glycerophosphate, magnesium glycinate, magnesium lactate, magnesium levulinate, magnesium malate, magnesium orotate, magnesium pidolate, and magnesium taurate. In some embodiments, the magnesium is magnesium malate, for example dimagnesium malate. In some embodiments, the magnesium is in the form of magnesium oxide. In some embodiments, the nutraceutical comprises magnesium oxide encapsulated within a phospholipid membrane. For example, in some embodiments, the nutraceutical comprises a magnesium-sucrosome compound, wherein magnesium oxide is encapsulated within a sucrosome particle, or a plurality of sucrosome particles. The sucrosome particle may comprise sucrose esters of fatty acids. The magnesium, such as magnesium oxide, may be encapsulated within the particle, or plurality of particles. In some embodiments, the sucrosome particle further comprises a calcium phosphate, such as tricalcium phosphate. In some embodiments, the sucrosome particle further comprises lecithin. The use of sucrosome particles to encapsulate magnesium may improve absorption of the magnesium by the digestive system (e.g., the small intestine) compared to magnesium which is not encapsulated. One example of such an encapsulated magnesium compound is Sucrosomial® magnesium. In another embodiment, the magnesium is in the form of dimagnesium malate.

In some embodiments, the nutraceutical comprises iron. The iron may be present in any suitable form, for example as an iron compound. In some embodiments, the iron is in the form of an iron salt. Iron salts may include salts of iron and amino acids or organic acids. Iron salts may include ferrous bisglycinate, ferric bisglycinate, ferrous fumarate, ferrous gluconate, ferrous lactate, or ferrous succinate. For example, in some embodiments, the nutraceutical comprises ferrous bisglycinate. The use of ferrous bisglycinate may increase absorption of iron by the digestive system (e.g., the small intestine), reduce nausea and other adverse side effects associated with consuming iron, or both, as compared to other forms of iron. In some embodiments, the iron is present as an iron-sucrosome compound, wherein the iron is encapsulated within a sucrosome particle, or plurality of sucrosome particles, as described above. In some embodiments, the encapsulated iron is an iron salt.

In some embodiments, the nutraceutical comprises zinc. The zinc may be present in any suitable form, for example a zinc compound. In some embodiments, the zinc is in the form of a zinc salt. Zinc salts may include zinc acetate, zinc ascorbate, zinc carnosinate, zinc citrate, zinc glycerophosphate, zinc glycinate, zinc lactate, zinc mono-L-methionate, and zinc succinate. In some embodiments, the zinc is in the form of zinc bisglycinate.

In some embodiments, the nutraceutical comprises calcium. The calcium may be present in any suitable form, for example as a calcium chelate or calcium salt. Calcium salts may include calcium acetate, calcium ascorbate, calcium citrate, calcium glycerophosphate, calcium glycinate, calcium lactate, calcium malate, and calcium citrate-malate. In some embodiments, the calcium is in the form of a calcareous marine algae extract. In some embodiments, the calcium is in the form of Lithothamnium spp. calcareous marine algae. In other embodiments, the calcium is in the form of a calcium-sucrosome compound. In some embodiments, the calcium is calcium bisglycinate.

In some embodiments, the nutraceutical comprises choline. The choline may be from any suitable source, for example as a choline salt or as a phospholipid. In some embodiments, the choline is in the form of choline bitartrate, choline chloride, or phosphatidylcholine. In some embodiments, the choline is derived from a natural source.

In some embodiments, the nutraceutical comprises iodine. The iodine may be present in any suitable form, for example as an iodine compound. In some embodiments, the iodine is an iodine salt, for example potassium iodide. In some embodiments, the iodine, such potassium iodide, is derived from kelp.

In some embodiments, the nutraceutical comprises selenium. The selenium may be present in any suitable form, for example as a selenium compound. In some embodiments, the selenium is in the form of L-selenocysteine or L-selenomethionine.

In some embodiments, the nutraceutical comprises chromium. The chromium may be present in any suitable form, for example as a chromium compound. In some embodiments, the chromium is in the form of a compound comprising Cr³⁺. For example, chromium may be present in the form of dinicocysteinate, nicotinate, or picolinate.

In some embodiments the nutraceutical comprises Vitamin C. The vitamin C may be from any suitable source. In some embodiments, the vitamin C is derived from a natural source, for example from citrus fruit, tomatoes, red peppers or potatoes. The vitamin C may be in any suitable form, for example as ascorbic acid, an ascorbate salt, or an ascorbate chelate.

In some embodiments the nutraceutical comprises Vitamin A. The vitamin A may be from any suitable source. In some embodiments, the vitamin A is derived from a natural source, for example from sweet potato, carrots, spinach, pumpkin, or mango. The vitamin A may be in any suitable form, for example as retinol, a retinyl ester, a provitamin A (e.g., alpha-carotene or beta-carotene).

In some embodiments, the tablet comprises at least one, at least two, at least three, at least four, at least five, at least six, at least seven, or at least eight nutraceuticals selected from the group consisting of B vitamins, boron, magnesium, iron, zinc, iodine, selenium, choline, chromium, probiotics, vitamin A, and vitamin C. In some embodiments, the tablet comprises at least one, at least two, at least three, at least four, or at least five nutraceuticals selected from the group consisting of B vitamins, boron, magnesium, vitamin A, and iron. The B vitamins may be, for example, vitamin B1, vitamin B6, vitamin B3, vitamin B12, or folate, or a combination thereof. In some embodiments, the B vitamins are vitamin B12 or folate, or a combination thereof. In some embodiments, the nutraceutical comprises vitamin B12, folate, boron, magnesium and iron. In other embodiments, the nutraceutical comprises vitamin B1, vitamin B6, vitamin B3, boron, magnesium, zinc, selenium, and chromium.

### (2) Prebiotics, Probiotics, and Postbiotics

In some embodiments, the nutraceutical comprises a probiotic. In some embodiments, the probiotic comprises a bacteria, a yeast, or a combination thereof. In some embodiments, the probiotics comprises a spore-forming species, a non-spore-forming species, or any combinations thereof. In some embodiments, the probiotics are spore-forming species. In another embodiment, the probiotics are non-spore-forming species. Probiotics are defined by their specific strain, which includes the genus, species, a subspecies (if applicable), and an alphanumeric strain designation. In some embodiments, the probiotics comprise a Bacillus species, or a Bacillus strains, or a combination thereof (see Office of Dietary Supplements. Probiotics: Fact Sheet for Health Professionals. NIH DHHS, 2020). In some embodiments, the probiotic is a microorganism of a genus selected from Lactobacillus, Lactiplantibacillus, Bifidobacterium, Saccharomyces, Streptococcus, Enterococcus, Escherichia, and Bacillus. In some embodiments, the probiotics comprise a Lactiplantibacillus species, or a Lactiplantibacillus strain, or a combination thereof. In some embodiments, the probiotic comprises a Lactiplantibacillus plantarum (strain designation 299v, formerly known as Lactobacillus plantarum 299v). In some embodiments, a probiotic comprises a cocci species, a cocci strains, or a combination thereof.

In some embodiments, the nutraceutical comprises a prebiotic. In some embodiments, the prebiotic comprises a nondigestible food component that selectively stimulate the growth or activity of desirable microorganisms (see, e.g., NIH NCCIH world wide web: nccih.nih.gov/health/probiotics-what-you-need-to-know). In some embodiments, the prebiotic improves host health. In some embodiments, the prebiotic is a compound in food that microorganisms in the gastrointestinal tract of a host use as metabolic fuel (see Gibson, et al (2017) Nat Rev Gastroenterol Hepatol 14:491). In some embodiments, the prebiotic comprises a carbohydrate. In some embodiments, the carbohydrate comprises a fructan. In some embodiments, the fructan is selected from inulin and fructo-oligosaccharide. In some embodiments, the carbohydrate comprises a galacto-oligosaccharide. In some embodiments, the prebiotic comprises starch. In some embodiments, the prebiotic comprises a flavanol. In some embodiments, the prebiotic comprises a bacteriophage blend.

In some embodiments, the nutraceutical comprises a postbiotic. In some embodiments, the postbiotic comprises a microbial metabolite. In some embodiments, the postbiotic comprises a short-chain fatty acid. In some embodiments, the postbiotic comprises a microbial metabolite.

### (3) Botanicals

In some embodiments, the nutraceutical comprises a botanical.

In some embodiments, the botanical is synthesized to be substantially similar to a compound obtained from a plant. In some embodiments, the botanical is an extract from a plant.

In some embodiments, the extracted matter is separated from other constituents present in the starting material. For example, in some embodiments, an extract is prepared by lyophilizing, dehydrating, or desiccating a plant material; then pulverizing or grinding the material to obtain a fine powder that is further passed through a sieve or fine mesh to obtain a uniform particulate. In some embodiments, the extract is further purified or processed following preparation, such as by soaking and heating the preparation (e.g., in water and/or alcohol depending on solubility of compounds therein), agitating the preparation, cooling the resulting liquid, straining, filtering, and removing unwanted products, then evaporating the liquid to obtain a concentrate or using a spray dryer to create a purified dried powder. A starch or other carrier can be added to the purified powder to improve formulation properties.

In some embodiments, the botanical is derived from a source selected from one or any combination set forth in Table 1. In some embodiments, the botanical comprises curcumin. In some embodiments, the botanical comprises ginseng. In some embodiments, the botanical comprises rhodiola. In some embodiments, the botanical comprises saffron. In some embodiments the botanical comprises ashwagandha.

TABLE 1
Exemplary Sources of Botanical Ingredients

| | | | |
|---|---|---|---|
| Alfalfa Herb and | Broccoli | Cornsilk Style and | Ginkgo Leaf |
| Leaf | | Stigma | |
| Aloe Vera Leaf | Brussel Sprouts | Cranberry | Goldenseal Root |
| Anise Seed | Buckthorn Bark | Damiana Leaf | Green Tea Leaf |
| Apple Cider Vinegar | Burdock Root | Dandelion Root | Guarana Seed |
| Apple Fruit | Cape Aloe Leaf | Devil's Claw Root | Guayusa Leaf |
| Arugula Leaf | Capsicum Fruit | Dong Quai Root | Gymnema Sylvestre |
| | | | Leaf |
| Ashwagandha Root | Carrot Root | Dulse Seaweed | Hawthorn Berry |
| Asparagus Stem | Cascara Sagrada | Echinacea Herb and | Hemp Herb |
| | Bark | Root | |
| Astragalus Root | Cat's Claw Bark | Eleuthero Root | Hibiscus Flower |
| Beet Root | Chamomile Flower | Fennel Seed | Hops Flower |
| Black Cohosh Root | Chia Seed | Fenugreek Seed | Horseradish Root |
| Black Walnut Hulls | Chickweed Herb | Fo-Ti Root | Horsetail Herb |
| Bladderwrack Plant | Cinnamon Bark | Ginger Root | Juniper Berry |
| Kale Leaf | Mullein Leaf | Purple Tea | Sweet Potato |
| Kelp Seaweed | Nettle Stinging Leaf | Quinoa Seed | Turmeric Root |
| Kola Nut | Noni Fruit | Red Clover Top | Uva Ursi Leaf |
| Kombucha | Nopalactin Leaf | Red Raspberry Leaf | Valerian Root |
| Licorice Root | Papaya Fruit | Rhubarb Root | Watercress Herb |
| Lucuma Fruit | Parsley Leaf | Rose Hips Fruit | White Willow Bark |
| Maca Root | Passionflower | Sage Leaf | Wild Yam Root |
| Marshmallow Root | Pau D'Arco Bark | Sarsaparilla Root | Spinach Leaf |
| Pumpkin Seed | Peppermint Leaf | Senna Leaf | Psyllium Seed Husk |
| Slippery Elm Bark | Tart Cherry | Lemon Balm Leaf | Saffron Flower |
| Ashwagandha Root | Rhodiola rosea | Sceletium tortuosum | Bacopa monnieri |
| & Leaf | | | |
| Green oat | Panax ginseng | Larch | Billygoat weed |
| | | arabinogalactan | |
| Pomegranate juice | Banana flower | Millet seed | Saw palmetto |
| Melon juice | Spearmint | Grape Fruit (Vitis | Lowbush Blueberry |
| | | vinifera L.) | Fruit (Vaccinium |
| | | | angustifolium A.) |
| Elderberry Fruit | | | |

In some embodiments, the nutraceutical comprises a botanical extract. In some embodiments, the botanical extract is one or any combination set forth in Table 2.

TABLE 2
Exemplary Botanical Extracts

| | | | |
|---|---|---|---|
| Acai Berry P.E. 4:1 | Dong Quai Root P.E. | Guarana Seed P.E. | Tamarind Fruit P.E. |
| | 4:1 | 12%, 22%, 36%, and | and S.E. |
| | | 50% Caffeine | |
| Acerola Fruit P.E. | Elderberry P.E. | Guayusa Leaf P.E. | Valerian Root P.E. |
| 15% and 25% | | | 4:1 |
| Vitamin C | | | |
| Acerola Fruit P.E. | Eleuthero Root P.E. | Hemp P.E. (Full | Valerian Root P.E. |
| 4:1 | 4:1 and 5:1 | Spectrum) | 0.8% Valerenic |
| | | | Acids |
| Aloe Vera Leaf Gel | Ginger Root P.E. 4:1 | Hemp Soft Extract | White Vinegar |
| 200:1 Concentrate | 6% Volatile Oil | | (Distilled) P.E. |
| Apple Cider Vinegar | Ginseng (American) | Lucuma Fruit P.E. | White Willow Bark |
| P.E. | Root P.E. 5% | | P.E. 5:1 |
| | Ginsenosides | | |
| Apple Cider Vinegar | Ginseng (Panax) | Marshmallow Root | Yerba Mate Leaf P.E. |
| P.E. 1% and 35% | Root P.E. and P.E. | P.E. 4:1 | 4:1 8% Caffeine |
| Acetic Acid | 4:1 | | |
| Arugula Leaf P.E. | Ginseng (Panax) | Milk Thistle Seed | Passionflower P.E. |
| | Root P.E. 7% and 8% | P.E. 80% Silymarin | 4% Flavonoids |
| | Ginsenosides | | |
| Blueberry P.E. 5:1 | Grape Seed P.E. 95% | Rose Hips Fruit P.E. | Paw Paw Twig P.E. |
| | OPC | 4:1 | |
| Boswellia Serrata | Green Tea Leaf P.E. | Rosemary Leaf P.E. | Pomegranate Fruit |
| Oleo-Gum Resin P.E. | 15% EGCG 8% | 6% Carnosic Acid | P.E. 3:1 |
| 65% Boswellic Acid | Caffeine | (RoseOx ® Basic) | |
| Celery Seed P.E. 6:1 | Green Tea Leaf P.E. | Saw Palmetto Berry | Pomegranate Juice |
| 2.2% Volatile Oil | 60% EGCG | P.E. 45% Fatty Acids | P.E. |
| Acerola Fruit P.E. | Green Tea Leaf P.E. | St. John's Wort Herb | Green Tea Leaf P.E. |
| 15% and 25% | 80% Polyphenols | P.E. 0.3% Hypericin | 98% Polyphenols |
| Vitamin C | 60% Catechins 30% | | 75% Catechins 45% |
| | EGCG Decaffeinated | | EGCG |
| Chamomile Flower | Kola Nut P.E. 12% | Chickpea P.E. | Cinnamon Bark P.E. |
| P.E. 4:1 | Caffeine | | 4:1 |
| Kombucha P.E. | Lemon Balm Leaf | Damiana Leaf P.E. | *P.E. = powdered |
| | P.E. | | extract |

In some embodiments, the nutraceutical comprises a botanical oil. In some embodiments, the botanical oil comprises a nut oil. In some embodiments, the botanical oil comprises a citrus oil. In some embodiments, the citrus oil is selected from lemon oil, a grapefruit oil, and an orange oil. In some embodiments, the botanical oil comprises a herbal oil. In some embodiments, the herbal oil is selected from a peppermint oil, a thyme oil, a spearmint oil, a lavender oil, a cypress oil, a myrtle oil, a cumin oil, and a sage oil. In some embodiments, the botanical oil comprises a spice oil. In some embodiments, the spice oil is selected from anise oil, garlic oil, a coriander oil. In some embodiments, the botanical oil is selected from Rosalina oil, acai oil, argan oil, rosehip seed oil, and ginger oil.

### (4) Amino Acids

In some embodiments, the nutraceutical comprises an amino acid. In some embodiments, the amino acid is an essential amino acid. In some embodiments, the amino acid is an L-amino acid. In some embodiments, the amino acid is selected from histidine, isoleucine, leucine, lysine, methionine, phenylalanine, threonine, tryptophan, valine, and a combination thereof. In some embodiments, the amino acid is a non-essential amino acid. In some embodiments, the amino acid is selected from L-theanine, glycine, or a combination thereof.

### (5) Stimulants

In some embodiments, the nutraceutical comprises a stimulant. In some embodiments, the stimulant is selected from caffeine, guarana, and a combination thereof. In some embodiments, the stimulant is selected from caffeine, TeaCrine, or a combination thereof.

### (6) Nootropic

In some embodiments, the nutraceutical comprises a nootropic. In some embodiments, the benefit for cognition comprises increased attention. In some embodiments, the benefit for cognition comprises increased responsiveness to stimuli. In some embodiments, the benefit for cognition comprises increased rate of problem-solving. In some embodiments, the benefit for cognition comprises improved memory. In some embodiments, the improved cognition is observed in a user having impaired cognition.

In some embodiments, the nootropic functions by enhancing release of a neurotransmitter. In some embodiments, the nootropic functions by improving blood flow to the brain. In some embodiments, the nootropic functions by improving supply of glucose and/or oxygen to the brain. In some embodiments, the nootropic functions by stimulating metabolism in neurons. In some embodiments, the nootropic functions by stimulating production of neurohormones. In some embodiments, the nootropic functions by reducing oxygen free radicals in the central nervous system.

In some embodiments, the nutraceutical comprises a non-stimulant nootropic. In some embodiments, the nootropic (e.g., the non-stimulant nootropic) is selected from the group consisting of phosphatidylserine, green oat extract, citicoline, pycnogenol, sceletium tortuosum, bacopa monnieri, Rhodiola rosea, Ginkgo biloba, Panax ginseng, spearmint extract, French grape extract (e.g., Vitis vinifera L. extract), North-American wild blueberry extract (e.g., Vaccinium angustifolium A. extract), and a combination thereof.

In some embodiments, the nootropic (e.g., the non-stimulant nootropic) comprises a saffron extract, L-theanine, GABA, lemon balm extract, magnesium, or a combination thereof.

### Excipients

In some embodiments, the excipient comprises a binder, a lubricant, a flow aid, a colorant, a rate-controlling polymer, a lubricant, a disintegrant, a glidant, and a combination thereof.

In some embodiments, the excipient comprises a binder. In some embodiments, the binder is a natural polymer, e.g., a natural polymer selected from Arabic gum, gelatin, sodium alginate, pullulan, starch, pregelatinized starch, and tragant. In some embodiments, the binder is a semi-synthetic polymer, e.g., a semi-synthetic polymer selected from Carboxymethylcellulose Sodium, Dextrin, Hydroxyethylcellulose, Hydroxypropylcellulose, Hypromellose, Maltodextrin, and Methylcellulose. In some embodiments, the binder is a synthetic polymer, e.g., a synthetic polymer selected from copovidone, a macrogols, a polyvinyl alcohols (PVA), and povidone. In some embodiments, the binder is selected from a Fatty alcohols, a Fat, a Wax, a Hydrated Rizinius Oil, and Stearic Acid. In some embodiments, the binder is selected from Povidone (Polyvinylpyrrolidone, PVP), Hydroxypropyl cellulose (HPC), Microcrystalline cellulose (MCC), Polyethylene glycol (PEG), Gelatin, Starch, a Carbomers and Sodium carboxymethyl cellulose (NaCMC). In some embodiments, the binder is MCC.

In some embodiments, the excipient comprises a lubricant. In some embodiments, the lubricant is selected from Magnesium stearate, Stearic acid, Calcium stearate, Sodium stearyl fumarate, Polyethylene glycols, Silicone dioxide, Talc, Beeswax, and Hydrogenated vegetable oil. In some embodiments, the lubricant comprises ascorbyl palmitate powder. In some embodiments, the glidant or the flow aid comprises calcium palmitate, magnesium stearate, fumed silica, starch, talc, or a combination thereof. In some embodiments, the glidant or flow aid comprises silica. In some embodiments, the lubricant comprises a hydrogenated vegetable oil. In some embodiments, the lubricant comprises sunflower oil. In some embodiments, the lubricant comprises MCT oil.

In some embodiments, the excipient comprises a disintegrant. In some embodiments, the disintegrant is selected from alginic acid, bentonite, MCC, powdered cellulose, guargalactomannan, and a combination thereof. In some embodiments, the disintegrant comprises a starch (e.g., corn starch, potato starch, pregelatinized starch, sodium starch glycolate, and/or starch 1500). In some embodiments, the starch is corn starch, rice starch, pea starch, tapioca starch, or a combination thereof. In some embodiments, the disintegrant comprises a cellulose or a derivative thereof (e.g., microcrystalline cellulose, croscarmellose sodium, sodium carboxymethyl cellulose, and hydroxypropyl methylcellulose). In some embodiments, the disintegrant comprises a gum (e.g., guar gum, xanthan gum, and locust bean gum). In some embodiments, the disintegrant comprises a calcium silicate (e.g., dicalcium phosphate or tricalcium phosphate). In some embodiments, the disintegrant comprises sodium alginate, cross-linked polyvinylpyrrolidone, and/or chitosan. In some embodiments, the disintegrant is selected from Carboxymethylcellulose Calcium, Carmellose Sodium, Croscarmellose Sodium, Sodium Starch Glycolate Type A or Type B, Low-Substituted Carboxymethylcellulose Sodium, Low-Substituted Hydroxypropyl Cellulose, and crospovidon. In some embodiments, the disintegrant comprises psyllium husk, oat fiber, and a combination thereof.

In some embodiments, the excipient comprises a flavor agent or odorant agent, or a combination thereof. A flavor agent may comprise one or more compounds that impart a flavor to the dietary supplement composition. An odorant agent comprises one or more compounds that impart an odor to the dietary supplement composition. In some embodiments, a flavor agent may also act as an odor agent. Including one or more flavor agents and/or odorant agents in the oil may increase compliance with a dosing regimen. The flavor agent or odorant agent may be derived from natural sources. In some embodiments, the odorant agent is a plant oil, a compound derived from plants, or an oil comprising one or more compounds derived from a plant. In some embodiments, the flavor agent is a plant oil, a compound derived from plants, or an oil comprising one or more compounds derived from a plant. For example, in certain embodiments, the flavor agent or odorant agent comprises a flavor, oil, extract, or scent selected from the group consisting of mint, vanilla, ginger, grapefruit, rosemary, lemon, lime, and orange. For example, the flavor agent or odorant agent may be vanilla flavor, mint flavor, mint oil, ginger flavor, ginger oil, grapefruit flavor, grapefruit oil, rosemary flavor, rosemary oil, lemon flavor, lemon oil, orange flavor, or orange oil. In one embodiment, the flavor agent or odorant agent comprises a mint oil. In one example, the mint oil is peppermint oil. In another embodiment, the flavor agent or odorant agent comprises vanilla flavor. In one embodiment, the vanilla flavor is derived from natural sources.

In some embodiments, the excipient comprises a colorant. In some embodiments, the colorant is selected from any known in the art (see, e.g., Biswal, et al (2015) IJPCBS 5:1004). In some embodiments, the colorant is naturally derived. In some embodiments, the colorant is selected from a Polyphenol derivative, an isoprene derivative, a Tetrapyrrole derivative, a Ketone derivative, a Quinone derivative, and a combination thereof. In some embodiments, the colorant comprises a carotenoid (e.g., capsanthin, crocin, lutein, bixin), an indol derivative (e.g., indigotin, bromoindigotin), a oxyindol glycoside (e.g., betanin), a diaryl heptanoid (e.g., curcumin), a benzopyrone (e.g., hacmatin). In some embodiments, the colorant comprises beta-carotene. In some embodiments, the colorant comprises indigotine. In some embodiments, the colorant comprises spirulina extract. In some embodiments, the colorant comprises saffron extract. In some embodiments, the colorant comprises riboflavin. In some embodiments, the colorant comprises turmeric. In some embodiments, the colorant comprises purple carrot.

In some embodiments, the excipient comprises a rate-controlling polymer. In some embodiments, the rate-controlling polymer comprises polyethylene glycol (PEG). In some embodiments, the PEG comprises a molecular weight of about 400 to about 6000 Da. In some embodiments, the rate controlling polymer comprises poly (lactic co-glycolic acid). In some embodiments, the rate controlling polymer comprises cellulose acetate. In some embodiments, the rate controlling polymer comprises polyacrylic acid. In some embodiments, the rate controlling polymer comprises polylactic acid. In some embodiments, the rate controlling polymer comprises polyethylene oxide. In some embodiments, the rate-controlling polymer is selected from hydroxy propyl methyl cellulose (HMPC), hydroxypropyl cellulose (HPC), microcrystalline cellulose (MCC), and a combination thereof. In some embodiments, the rate controlling polymer comprises HMPC. In some embodiments, the HMPC is any grade known in the art. In some embodiments, the rate controlling polymer comprises HPMC grade K4M. In some embodiments, the rate controlling polymer comprises HPMC grade K100M. In some embodiments, the rate controlling polymer comprises HPMC grade K15M.

### Coating

In some embodiments, the tablets of the disclosure comprise a coating surrounding the matrix. In some embodiments, the coating comprises an excipient (e.g., 1, 2, 3, 4, or more excipients).

In some embodiments, the coating comprises up to 95 wt %, up to 98 wt %, up to 99 wt %, up to 99.5 wt %, up to 99.9 wt %, or up to 99.99 wt % of an excipient (e.g., 1, 2, 3, 4, or more excipients). In some embodiments, the coating does not comprise a nutraceutical.

In some embodiments, the coating comprises a polymer (e.g., a rate controlling polymer). In some embodiments, the polymer comprises a cellulose derivative (e.g., sodium carboxymethylcellulose, cellulose acetate phthalate, HPMC, HPC, cellulose acetate trimellate, HPMC acetate succinate, and/or ethyl cellulose). In some embodiments, the cellulose derivative is hydroxyethyl cellulose. In some embodiments, the cellulose derivative is HPC. In some embodiments, the cellulose derivative is HPMC. In some embodiments, the cellulose derivative is HPC. In some embodiments, the polymer comprises polyvinyl pyrrolidone. In some embodiments, the polymer comprises zein. In some embodiments, the polymer comprises an Eudragit polymer (e.g., Eudragit L-100-55).

In some embodiments, the coating comprises a vegetable oil. In some embodiments, the oil is sunflower oil. In some embodiments, the oil is MCT oil.

In some embodiments, the coating comprises an alginate. In some embodiments, the coating comprises sodium alginate.

In some embodiments, the polymer comprises a colorant. In some embodiments, the coating comprises a natural colorant (e.g., beta-carotene, riboflavin, spirulina, carmine lake). In some embodiments, the coating comprises an inorganic pigment, a water soluble dye, a D&C lake, and/or a FD&C lake.

### Capsules

In some embodiments, the solid dosage form comprises an extended release capsules that delays or extends the release of the plurality of tablets encapsulated therein. For example, an extended release capsule may prevent dissolution of the tablets in the stomach, such that the majority or all of the contents of the tablets are released in the digestive system after the capsule passes through the stomach. The use of a delayed release capsule may increase absorption of the nutraceutical(s) contained in the tablet(s), thereby decreasing unwanted interactions of the nutraceutical(s) contained therein by relating them where they are optimally absorbed in the gastrointestinal tract. Releasing nutraceuticals past the stomach may prevent exposure of the nutraceuticals to the low pH of the stomach, which can damage certain nutraceuticals or cause them to undergo one or more chemical reactions. In addition, stomach acid can cause unwanted interactions between one or more nutraceuticals or excipients that do not occur at the pH of the small intestine.

In some embodiments, the capsule is configured to bypass the stomach of the subject. In some embodiments, dissolution of the capsule is time dependent, releasing the ingredients after at least 10, 20, 30, 40, 50, 60, 70, 80, 90, or 100 or more minutes after ingestion. In some embodiments, the capsule is configured to release its contents after entering the intestine of the subject. In some embodiments, the capsule is configured to release its contents after entering the small intestine of the subject. In some embodiments, the capsule is configured to bypass the stomach of the subject and release its contents after entering the intestine of the subject.

In some embodiments, the solid dosage form comprises an immediate release capsule.

In some embodiments, the capsule comprises a polymers, such as hydroxypropyl methylcellulose (hypromellose), gellan gum, pullalan, or any combinations thereof. In some embodiments, the capsule comprises an excipient, such as one or more binders, fillers, diluents, lubricants, or any combinations thereof. In some embodiments, the capsule comprises hypromellose, gellan gum, and silica. In some embodiments, the capsule consists essentially of hypromellose, gellan gum, and silica.

The outer capsule may be any size suitable for containing the plurality of tablets described herein (e.g., 2, 3, 4, or more tablets). In some embodiments, the capsule is size 0. In some embodiments, the capsule is size 00. In some embodiments, the capsule is size 1. In some embodiments, the capsule is size 2.

In some embodiments, the capsule is essentially free of ingredients derived from animals. For example, the one or more capsules may comprise less than 1 wt %, less than 0.1 wt %, or less than 0.01 wt % gelatin. In some embodiments, the capsule is essentially free of gelatin.

In some embodiments, the capsule is translucent. In some embodiments, the capsule is semi-opaque. In some embodiments, the capsule is opaque.

### Exemplary Solid Dosage Forms

### (1) Formulations for Improving Sleep

In some embodiments, the disclosure provides a solid dosage form formulated to improve sleep. In some embodiments, the solid dosage form comprises a plurality of spherical tablets (e.g., 2, 3, 4, or more spherical tablets) in a single capsule, wherein at least one spherical tablet of the plurality comprises a dose of melatonin. In some embodiments, the solid dosage form comprises three spherical tablets in a single capsule, wherein at least one of the spherical tablets comprises a dose of melatonin (e.g., a dose of about 0.5 mg to about 5 mg melatonin). In some embodiments, the solid dosage form comprises three spherical tablets in a single capsule, wherein each of the spherical tablets comprises a dose of melatonin (e.g., a dose of about 0.5 mg to about 5 mg melatonin). In some embodiments, the solid dosage form is formulated for consumption in a dosing regimen comprising one capsule taken once per day. In some embodiments, the solid dosage form is formulated for consumption about 10 minutes, about 20 minutes, about 30 minutes, about 40 minutes, about 50 minutes, about 60 minutes, about 90 minutes, or about 120 minutes prior to a nocturnal sleep period. In some embodiments, the solid dosage form is formulated for consumption about 1 hour prior to a nocturnal sleep period.

Melatonin is a hormone produced by the pineal gland in humans and functions to regulate circadian rhythm. Melatonin supplementation in humans has been found to reduce jet lag (see, e.g., Herxheimer, et al (2002) Cochrane Database Systematic Rev 2: CD001520) and reduce sleep onset latency (see, e.g., Brzezinski et al (2005) Sleep Medicine Review 9:41). However, available melatonin supplements typically have release profiles that don't match the endogenous melatonin circadian rhythm. As appreciated by the skilled artisan, the endogenous melatonin circadian rhythm on average in a healthy human adult increases shortly after dark, reaches a peak in the middle of the night (e.g., between 2-4 AM), and declines throughout the rest of the night to reach a low point shortly before an individual awakens (see Grivas T B and Savvidou O D. Scoliosis. 2007 2:6).

Supplements formulated for instant release melatonin provide a rapid burst of melatonin that reduces time required for an individual to fall asleep. However, and without being bound by theory, as exogenously administered melatonin is rapidly processed and removed from the body (see Harpsoe N, et al. Eur J Clin Pharmacol. 2015 71 (8): 901-909), the bioavailability of melatonin is insufficient to support restful sleep throughout the duration of the night. Extended release melatonin supplements are typically formulated to provide extended bioavailability of melatonin, but blood melatonin levels may remain elevated beyond the typical sleep period (see Gooneratne N, et al. J Pincal Res. 2012 52 (4): 437-45). This may result in the user waking up with supraphysiological levels of melatonin in their blood in the morning, causing unwanted side effects, e.g., difficulty waking up and grogginess. As described herein, the disclosure provides melatonin-containing dosage forms designed to release melatonin following consumption in a manner that mimics an endogenous circadian rhythm for melatonin release, thereby promoting sleep support without substantial side effects upon waking.

In some embodiments, the solid dosage form comprises a plurality of spherical tablets (e.g., 2, 3, 4, or more spherical tablets) in a single capsule, wherein each spherical tablet of the plurality comprises a dose of melatonin (e.g., a dose of about 0.5 mg to about 5 mg melatonin), and wherein at least one spherical tablet of the plurality is formulated for immediate release and at least one spherical tablet of the plurality is formulated for extended release, wherein the at least one spherical tablet formulated for extended release comprises a rate-controlling polymer. In some embodiments, the solid dosage form comprises three spherical tablets in a single capsule, wherein each spherical tablet comprises a dose of melatonin (e.g., a dose of about 0.5 mg to about 5 mg melatonin), and wherein at least one spherical tablet of the plurality is formulated for immediate release, and wherein at least one spherical tablet of the plurality is formulated for extended release, said spherical tablet formulated for extended release comprising a rate-controlling polymer. In some embodiments, the solid dosage form comprises three spherical tablets in a single capsule, wherein each spherical tablet comprises a dose of melatonin (e.g., a dose of about 0.5 mg to about 5 mg melatonin), wherein a first spherical tablet is formulated for immediate release, a second spherical tablet is formulated for extended release, and a third spherical tablet is formulated for extended release, wherein the second spherical tablet and the third spherical tablet each comprise a rate-controlling polymer. In some embodiments, the second spherical tablet and the third spherical tablet are characterized by the same or different rates of dissolution of their respective doses of melatonin.

In some embodiments, the dose of melatonin is about 0.5 mg to about 1 mg, about 0.5 mg to about 1.5 mg, about 0.5 mg to about 2 mg, about 0.5 mg to about 2.5 mg, about 0.5 mg to about 3 mg, about 0.5 mg to about 3.5 mg, about 0.5 mg to about 4 mg, about 0.5 mg to about 4.5 mg, about 0.5 mg to about 5 mg, about 1 mg to about 1.5 mg, about 1 mg to about 2 mg, about 1 mg to about 2.5 mg, about 1 mg to about 3 mg, about 1 mg to about 3.5 mg, about 1 mg to about 4 mg, about 1 mg to about 4.5 mg, about 1 mg to about 5 mg, about 2 mg to about 2.5 mg, about 2 mg to about 3 mg, about 2 mg to about 3.5 mg, about 2 mg to about 4 mg, about 2 mg to about 4.5 mg, about 2 mg to about 5 mg, about 3 mg to about 3.5 mg, about 3 mg to about 4 mg, about 3 mg to about 4.5 mg, about 3 mg to about 5 mg, about 4 mg to about 4.5 mg, or about 4 mg to about 5 mg. In some embodiments, the dose of melatonin is about 0.5 mg to about 2 mg. In some embodiments, the dose of melatonin is about 2 mg to about 4 mg. In some embodiments, the dose of melatonin is about 1 mg. In some embodiments, the dose of melatonin is about 3 mg.

In some embodiments, the solid dosage form comprises three spherical tablets in a single capsule, wherein each spherical tablet comprises a dose of melatonin (e.g., a dose of about 0.5 mg to about 5 mg melatonin), wherein a first spherical tablet is formulated for immediate release and comprises a dose of about 0.5 mg to about 2 mg melatonin (e.g., about 1 mg melatonin), a second spherical tablet is formulated for extended release and comprises a dose of about 2 mg to about 4 mg (e.g., about 3 mg melatonin), and a third spherical tablet is formulated for extended release and comprises a dose of about 0.5 mg to about 2 mg melatonin (e.g., about 1 mg melatonin), wherein the second spherical tablet and the third spherical tablet each comprise a rate-controlling polymer. In some embodiments, the solid dosage form comprises three spherical tablets (e.g., 2, 3, 4, or more spherical tablets) in a single capsule, wherein each spherical tablet comprises a dose of melatonin (e.g., a dose of about 0.5 mg to about 5 mg melatonin), wherein a first spherical tablet is formulated for immediate release and comprises a dose of about 1 mg melatonin, a second spherical tablet is formulated for extended release and comprises a dose of about 3 mg melatonin, and a third spherical tablet is formulated for extended release and comprises a dose of about 1 mg melatonin, wherein the second spherical tablet and the third spherical tablet each comprise a rate-controlling polymer.

It should be understood that if the solid dosage form comprises, for example, a dose of 3 mg of melatonin, the amount of melatonin present in the solid dosage form may exceed 3 mg to account for e.g., loss during manufacturing. Accordingly, in some embodiments, the spherical tablet of the foregoing solid dosage form comprises an amount of melatonin that is greater than the dose of melatonin. In some embodiments, the spherical tablet comprises an amount of melatonin that is about 1.1-fold, about 1.2-fold, about 1.3-fold, about 1.4-fold, or about 1.5-fold greater than the dose of melatonin.

In some embodiments, the spherical tablet formulated for extended release releases the dose of melatonin in about 1 hour to about 12 hours under gastrointestinal dissolution conditions. In some embodiments, the spherical tablet formulated for extended release releases the dose of melatonin in about 3 hours to about 12 hours under gastrointestinal dissolution conditions. In some embodiments, the spherical tablet formulated for extended release releases the dose of melatonin in about 3 hours to about 9 hours under gastrointestinal dissolution conditions. In some embodiments, the spherical tablet formulated for extended release releases the dose of melatonin in about 2 hours to about 6 hours under gastrointestinal dissolution conditions. In some embodiments, the spherical tablet formulated for extended release releases the dose of melatonin in about 4 hours to about 8 hours under gastrointestinal dissolution conditions. In some embodiments, the spherical tablet formulated for extended release releases the dose of melatonin in about 6 hours to about 8 hours under gastrointestinal dissolution conditions. In some embodiments, the spherical tablet formulated for extended release releases the dose of melatonin in about 5 hours to about 7 hours under gastrointestinal dissolution conditions. In some embodiments, the spherical tablet formulated for extended release releases the dose of melatonin in about 5 hours, about 6 hours, about 7 hours, or about 8 hours under gastrointestinal dissolution conditions. In some embodiments, the spherical tablet formulated for extended release releases the dose of melatonin in about 2 hours, about 3 hours, about 4 hours, or about 5 hours under gastrointestinal dissolution conditions.

In some embodiments, the spherical tablet formulated for immediate release releases the dose of melatonin in less than about 1 hour under gastrointestinal dissolution conditions. In some embodiments, the spherical tablet formulated for immediate release releases the dose of melatonin in about 5 minutes to about 60 minutes under gastrointestinal dissolution conditions. In some embodiments, the spherical tablet formulated for immediate release releases the dose of melatonin in about 5 minutes, about 10 minutes, about 15 minutes, about 20 minutes, about 25 minutes, about 30 minutes, about 35 minutes, about 40 minutes, about 45 minutes, about 50 minutes, about 55 minutes or about 60 minutes under gastrointestinal dissolution conditions.

In some embodiments, the spherical tablet formulated for extended release comprises a rate controlling polymer in an amount. In some embodiments, the amount of the rate controlling polymer of the tablet formulated for extended release is about 5% to about 15% by weight of the tablet. In some embodiments, the amount is about 5%, about 6%, about 7%, about 8%, about 9%, about 10%, about 11%, about 12%, about 13%, about 14%, or about 15% by weight of the tablet. In some embodiments, the spherical tablet formulated for extended release comprises a coating, wherein the coating comprises a rate controlling polymer in an amount. In some embodiments, the amount of the rate controlling polymer of the coating formulated for extended release is about 2% to about 10% by weight of the tablet. In some embodiments, the amount is about 5%, about 6%, about 7%, about 8%, about 9%, or about 10% by weight of the tablet. In some embodiments, the rate controlling polymer comprises HPMC, HPC, MCC, and a combination thereof. In some embodiments, the rate controlling polymer comprises HPMC, HPC, and MCC. In some embodiments, the HPMC is a grade E15 HPMC, a grade K4M HPMC, or a combination thereof.

In some embodiments, the spherical tablet formulated for immediate release comprises a disintegrant in an amount. In some embodiments, the amount of the disintegrant of the tablet formulated for immediate release is about 0.1% to about 5% by weight of the tablet. In some embodiments, the amount of the disintegrant of the tablet formulated for immediate release is about 0.1%, about 0.5%, about 1%, about 1.5%, about 2%, about 2.5%, about 3%, about 3.5%, about 4%, about 4.5%, or about 5% by weight of the tablet. In some embodiments, the disintegrant comprises psyllium husk, oat fiber, or a combination thereof.

In some embodiments, at least one spherical tablet of the solid dosage form comprises a colorant. In some embodiments, at least one spherical tablet of the solid dosage form does not comprise a colorant. In some embodiments, the spherical tablets of the solid dosage form are substantially similar in color. In some embodiments, the spherical tablets of the solid dosage form are substantially different in color. In some embodiments, the colorant comprises spirulina extract.

In some embodiments, at least one spherical tablet of the solid dosage form comprises a color. In some embodiments, at least two spherical tablets of the solid dosage form comprise a color, wherein the color is the same or different. In some embodiments, at least two spherical tablets of the solid dosage form comprise a color, wherein the color is the same, and wherein the shade of the at least two spherical tablets is the same or different. In some embodiments, at least two spherical tablets of the solid dosage form comprise a color, wherein the color is the same, wherein at least one spherical tablet is a darker shade of the color and at least one spherical tablet is a lighter shade of the color. In some embodiments, at least one spherical tablet of the dosage form is a darker shade of the color, at least one spherical tablet of the dosage form is a lighter shade of the color, and at least one spherical tablet of the dosage form lacks a colorant. In some embodiments, the color is blue. In some embodiments, at least one spherical tablet of the dosage form is a darker shade of blue, at least one spherical tablet of the dosage form is a lighter shade of blue, and at least one spherical tablet is white.

In some embodiments, the solid dosage form comprises three spherical tablets in a single capsule, wherein each spherical tablet comprises a dose of melatonin, wherein the spherical tablets comprise an immediate release spherical tablet, a first extended release spherical tablet, and a second extended release spherical tablet; wherein the immediate release spherical tablet comprising a dose of about 0.5 mg to about 2 mg melatonin (e.g., about 1 mg melatonin), the first extended release spherical tablet comprising a dose of about 2 mg to about 4 mg (e.g., about 3 mg melatonin), and the second extended release spherical tablet comprising a dose of about 0.5 mg to about 2 mg melatonin (e.g., about 1 mg melatonin), wherein the immediate release spherical tablet is formulated to release at least about 85% of the dose of melatonin contained therein in less than about 2 hours, the first extended release spherical tablet is formulated to release at least about 85% of the dose of melatonin contained therein in about 6 hours to about 8 hours (e.g., about 6 hours), and the second extended release spherical tablet is formulated to release at least about 85% of the dose of melatonin contained therein in about in about 2 hours to about 4 hours (e.g., about 3 hours), each as measured upon exposure to gastrointestinal dissolution conditions or following oral administration. In some embodiments, the first extended release spherical tablet and the second extended release spherical tablet each comprise a rate-controlling polymer. In some embodiments, the first extended release spherical tablet and the second extended release spherical tablet each comprise an amount of the rate controlling polymer that is about 5% to about 15% by weight of the tablet (e.g., about 8% to about 12% by weight of the tablet). In some embodiments, the first extended release spherical tablet and the second extended release spherical tablet each comprise HPMC. In some embodiments, the first extended release spherical tablet and the second extended release spherical tablet each comprise an amount of the HPMC that is about 5% to about 15% by weight of the tablet (e.g., about 8% to about 12% by weight of the tablet). In some embodiments, the first extended release spherical tablet and the second extended release spherical tablet each comprise an about 2:1 to about 1:2 ratio (e.g., an about 1:1 ratio) of grade E15 HPMC and a grade K4M HPMC. In some embodiments, the first extended release spherical tablet and the second extended release spherical tablet each comprise an amount of grade E15 HPMC that is about 2% to about 10% by weight of the tablet (e.g., about 4% to about 6% by weight of the tablet) and an amount of grade K4M HPMC that is about 2% to about 10% by weight of the tablet (e.g., about 4% to about 6% by weight of the tablet), wherein the ratio of grade E15 HPMC to grade K4M HPMC is about 2:1 to about 1:2 (e.g., about 1:1). In some embodiments, the immediate release spherical tablet, the first extended release spherical tablet, and the second extended release spherical tablet are the same or different colors. Iin some embodiments, the immediate release spherical tablet is a darker shade of blue, the first extended release spherical tablet is a lighter shade of blue, and the second extended release spherical tablet is white.

In some embodiments, the solid dosage form comprises three spherical tablets in a single capsule, wherein each spherical tablet comprises a dose of melatonin, wherein the spherical tablets comprise an immediate release spherical tablet, an extended release spherical tablet, and an intermediate release spherical tablet; wherein the immediate release spherical tablet comprising a dose of about 0.5 mg to about 2 mg melatonin (e.g., about 1 mg melatonin), the extended release spherical tablet comprising a dose of about 2 mg to about 4 mg (e.g., about 3 mg melatonin), and the intermediate release spherical tablet comprising a dose of about 0.5 mg to about 2 mg melatonin (e.g., about 1 mg melatonin), wherein the immediate release spherical tablet is formulated to release at least about 85% of the dose of melatonin contained therein in less than about 2 hours, the extended release spherical tablet is formulated to release at least about 85% of the dose of melatonin contained therein in about 6 hours to about 8 hours (e.g., about 6 hours), and the intermediate release spherical tablet is formulated to release at least about 85% of the dose of melatonin contained therein in about in about 2 hours to about 4 hours (e.g., about 3 hours), each as measured upon exposure to gastrointestinal dissolution conditions or following oral administration. In some embodiments, the extended release spherical tablet and the intermediate release spherical tablet each comprise a rate-controlling polymer. In some embodiments, the extended release spherical tablet and the intermediate release spherical tablet each comprise an amount of the rate controlling polymer that is about 5% to about 15% by weight of the tablet (e.g., about 8% to about 12% by weight of the tablet). In some embodiments, the extended release spherical tablet and the intermediate release spherical tablet each comprise HPMC. In some embodiments, the extended release spherical tablet and the intermediate release spherical tablet each comprise an amount of the HPMC that is about 5% to about 15% by weight of the tablet (e.g., about 8% to about 12% by weight of the tablet). In some embodiments, the extended release spherical tablet and the intermediate release spherical tablet each comprise an about 2:1 to about 1:2 ratio (e.g., an about 1:1 ratio) of grade E15 HPMC and a grade K4M HPMC. In some embodiments, the extended release spherical tablet and the intermediate release spherical tablet each comprise an amount of grade E15 HPMC that is about 2% to about 10% by weight of the tablet (e.g., about 4% to about 6% by weight of the tablet) and an amount of grade K4M HPMC that is about 2% to about 10% by weight of the tablet (e.g., about 4% to about 6% by weight of the tablet), wherein the ratio of grade E15 HPMC to grade K4M HPMC is about 2:1 to about 1:2 (e.g., about 1:1). In some embodiments, the immediate release spherical tablet, the extended release spherical tablet, and the intermediate release spherical tablet are the same or different colors. In some embodiments, the immediate release spherical tablet is a darker shade of blue, the extended release spherical tablet is a lighter shade of blue, and the intermediate release spherical tablet is white.

In some embodiments, the capsule of any of the foregoing solid dosage forms is formulated for instant release. In some embodiments, the capsule is formulated for delayed release. In some embodiments, the capsule is translucent. In some embodiments, the capsule comprises a rate controlling polymer and gellan gum. In some embodiments, the capsule comprises HPMC and gellan gum. In some embodiments, the capsule lacks an animal-derived ingredient. In some embodiments, the capsule is standard size 0. In some embodiments, the capsule is standard size 00. In some embodiments, the capsule is elongated size 0. In some embodiments, the capsule is elongated size 00. In some embodiments, the capsule is standard size 1. In some embodiments, the capsule is standard size 2.

In some embodiments, the spherical tablets of the foregoing dosage forms are substantially similar in size and/or shape. In some embodiments, the spherical tablets are a modified ball shape. In some embodiments, the modified ball shape comprises a diameter of about 6 mm to about 10 mm. In some embodiments, the modified ball shape comprises a diameter of about 6 mm to about 8 mm. In some embodiments, the modified ball shape comprises a diameter of about 6 mm to about 7 mm. In some embodiments, the modified ball shape comprises a diameter of about 6 mm, about 6.4 mm, about 6.5 mm, about 6.7 mm, about 6.9 mm, about 7 mm, or about 7.5 mm. In some embodiments, the modified ball shape comprises a diameter of about 6.4 mm. In some embodiments, the modified ball shape comprises a diameter of about 6.7 mm. In some embodiments, the modified ball shape comprises a diameter of about 6.9 mm. In some embodiments, the spherical tablet comprises a diameter of about 6 mm, about 6.4 mm, about 6.7 mm, or about 6.9 mm and the capsule is standard size 00.

In some embodiments, the capsule of any of the foregoing solid dosage forms is substantially filled by the spherical tablets. In some embodiments, the spherical tablets comprise a diameter and the capsule comprises a circular cross-section transverse to a longest dimension, wherein the diameter of the spherical tablets is about 90% to about 99% of the diameter of the circular cross-section. In some embodiments, the spherical tablets of the solid dosage form comprise a combined volume, wherein the combined volume is at least about 90% to about 99% of the volume of the capsule.

### (2) Formulations for Reducing Stress

In some embodiments, the disclosure provides a solid dosage form formulated to reduce stress. As understood by the skilled artisan, endogenous cortisol levels are regulated by a circadian rhythm. In healthy human adults, cortisol levels rise early in the morning, peak shortly after waking, decline throughout the rest of the day, and reach a low point in the middle of the night (see Jones C and Gwennin C. Physiol Rep. 2021 8 (24): e14644; Russel G and Lightman S. Nat Rev Endocrinol. 2019 15 (9): 525-534; Azmi et al (2021) Int J Environ Res Public Health 18:676).

As described herein, the disclosure provides solid dosage forms comprises one or more active ingredients for altering endogenous cortisol levels, wherein release of the active ingredients is designed to alter endogenous cortisol levels following consumption in a manner that mimics the circadian rhythm for endogenous cortisol. In some embodiments, the solid dosage form is formulated for consumption in a dosing regimen comprising one capsule taken once per day. In some embodiments, the capsule is consumed in the morning. Without being bound by theory, consumption of the capsule in the morning when cortisol levels are highest functions to normalize these levels in a manner that controls stress levels through the day.

In some embodiments, the solid dosage form comprises a plurality of spherical tablets (e.g., 2, 3, 4, or more spherical tablets) in a single capsule, wherein at least one spherical tablet of the plurality comprises a dose of a nutraceutical selected from L-theanine, saffron, ashwagandha, and a combination thereof. In some embodiments, the solid dosage form comprises a plurality of spherical tablets (e.g., 2, 3, 4, or more spherical tablets) in a single capsule, wherein each spherical tablet of the plurality comprises a dose of a nutraceutical selected from L-theanine, saffron, ashwagandha, and a combination thereof, wherein at least one spherical tablet of the plurality is formulated for immediate release and at least one spherical tablet of the plurality is formulated for extended release, and wherein the at least one spherical tablet formulated for extended release comprises a rate-controlling polymer. In some embodiments, the nutraceutical in the spherical tablets of the plurality is the same. In some embodiments, the nutraceutical in the spherical tablets of the plurality is different. In some embodiments, the dose of the nutraceutical in the spherical tablets of the plurality is the same. In some embodiments, the dose of the nutraceutical in the spherical tablets of the plurality is different. In some embodiments, at least one spherical tablet of the plurality comprises a dose of L-theanine. In some embodiments, at least one spherical tablet of the plurality comprises a dose of saffron. In some embodiments, at least one spherical tablet of the plurality comprises a dose of ashwagandha. In some embodiments, at least one spherical tablet of the plurality comprises a dose of L-theanine, a dose of saffron, and a dose of ashwagandha.

L-Theanine is an amino acid that has been shown to reduce stress in humans (see, e.g., Kimura et al (2007) Biol Psychol 74:39; Hidese, et al (2019) Nutrients 11:2362; Hidese, et al (Nutrients. 2019 Oct. 3; 11 (10): 2362). Results from several clinical trials suggest that ashwagandha extracts may help reduce stress and anxiety, as well as support normal cortisol levels (see, e.g., Lopresti, et al (2021) J. Herb Med 28:100434; Lopresti, et al (Medicine (Baltimore). 2019 Scp; 98 (37): e17186)). Saffron has been shown in several studies to improve mood and wellbeing in adults experiencing low mood and anxiety (see, e.g., Marx, et al (2019) Nutr Rev 77:557; Kell, et al (Complement Ther Med. 2017 August; 33:58-64); Lopresti, et al (2019) J Psychopharmacol 33:1415; and Lopresti, et al (2021) J Menopausal Med 27:66-78).

In some embodiments, the solid dosage form comprises three spherical tablets in a single capsule, wherein at least one of the spherical tablets comprises a dose of a nutraceutical selected from L-theanine, saffron, ashwagandha, and a combination thereof. In some embodiments, the solid dosage form comprises three spherical tablets in a single capsule, wherein each of the spherical tablets comprises a dose of a nutraceutical selected from L-theanine, saffron, ashwagandha, and a combination thereof. In some embodiments, the solid dosage form comprises three spherical tablets in a single capsule, wherein each spherical tablet comprises a dose of a nutraceutical selected from L-theanine, saffron, ashwagandha, and a combination thereof, and wherein at least one spherical tablet is formulated for immediate release, and wherein at least one spherical tablet of the plurality is formulated for extended release, said spherical tablet formulated for extended release comprising a rate-controlling polymer. In some embodiments, the solid dosage form comprises three spherical tablets in a single capsule, wherein each spherical tablet comprises a dose of a nutraceutical selected from L-theanine, saffron, ashwagandha, and a combination thereof, wherein a first spherical tablet is formulated for immediate release, a second spherical tablet is formulated for extended release, and a third spherical tablet is formulated for extended release, wherein the second spherical tablet and the third spherical tablet each comprise a rate-controlling polymer. In some embodiments, the solid dosage form comprises three spherical tablets in a single capsule, wherein each spherical tablet comprises a dose of a nutraceutical selected from L-theanine, saffron, ashwagandha, and a combination thereof, wherein a first spherical tablet is an immediate release tablet, a second spherical tablet is an intermediate release tablet, and a third spherical tablet is an extended release tablet, wherein the second spherical tablet and the third spherical tablet each comprise a rate-controlling polymer. In the embodiments, the nutraceutical in the three spherical tablets is the same. In the embodiments, the nutraceutical in the three spherical tablets is different. In the embodiments, the dose of the nutraceutical in the three spherical tablets is the same. In the embodiments, the dose of the nutraceutical in the three spherical tablets is different. In some embodiments, at least one of the three spherical tablets comprises a dose of L-theanine. In some embodiments, at least one of the three spherical tablets comprises a dose of saffron. In some embodiments, at least one of the three spherical tablets comprises a dose of ashwagandha. In some embodiments, at least one of the three spherical tablets comprises a dose of L-theanine, a dose of saffron, and a dose of ashwagandha. In some embodiments, each of the three spherical tablets comprises a dose of L-theanine, a dose of saffron, and a dose of ashwagandha.

In some embodiments, the dose of L-theanine is about 50 mg to about 100 mg, about 50 mg to about 90 mg, about 50 mg to about 80 mg, about 50 mg to about 70 mg, about 50 mg to about 60 mg, about 60 mg to about 100 mg, about 60 mg to about 90 mg, about 60 mg to about 80 mg, about 60 mg to about 70 mg, about 70 mg to about 100 mg, about 70 mg to about 90 mg, about 70 mg to about 80 mg, about 80 mg to about 100 mg, about 80 mg to about 90 mg, or about 90 mg to about 100 mg. In some embodiments, the dose of L-theanine is about 60 mg to about 100 mg. In some embodiments, the dose of L-theanine is about 60 mg to about 70 mg. In some embodiments, the dose of L-theanine is about 61 mg, about 62 mg, about 63 mg, about 64 mg, about 65 mg, about 66 mg, about 67 mg, about 68 mg, or about 69 mg.

In some embodiments, the dose of saffron is about 1 mg to about 25 mg, about 1 mg to about 20 mg, about 1 mg to about 15 mg, about 1 mg to about 10 mg, about 1 mg to about 8 mg, about 1 mg to about 5 mg, about 1 mg to about 4 mg, about 1 mg to about 3 mg, about 1 mg to about 2 mg, about 2 mg to about 25 mg, about 2 mg to about 20 mg, about 2 mg to about 15 mg, about 2 mg to about 10 mg, about 2 mg to about 8 mg, about 2 mg to about 5 mg, about 2 mg to about 4 mg, about 2 mg to about 3 mg, about 3 mg to about 25 mg, about 3 mg to about 20 mg, about 3 mg to about 15 mg, about 3 mg to about 10 mg, about 3 mg to about 8 mg, about 3 mg to about 5 mg, or about 3 mg to about 4 mg. In some embodiments, the dose of saffron is about 2 mg to about 10 mg. In some embodiments, the dose of saffron is about 8 mg to about 12 mg. In some embodiments, the dose of saffron is about 15 mg to about 20 mg.

In some embodiments, the dose of ashwagandha is about 20 mg to about 100 mg, about 20 mg to about 90 mg, about 20 mg to about 80 mg, about 20 mg to about 70 mg, about 20 mg to about 60 mg, about 20 mg to about 50 mg, about 20 mg to about 40 mg, about 30 mg to about 100 mg, about 30 mg to about 90 mg, about 30 mg to about 80 mg, about 30 mg to about 70 mg, about 30 mg to about 60 mg, about 30 mg to about 50 mg, or about 30 mg to about 40 mg. In some embodiments, the dose of ashwagandha is about 20 mg to about 40 mg. In some embodiments, the dose of ashwagandha is about 25 mg to about 35 mg. In some embodiments, the dose of ashwagandha is about 20 mg, about 21 mg, about 22 mg, about 23 mg, about 24 mg, about 25 mg, about 26 mg, about 27 mg, about 28 mg, or about 29 mg.

In some embodiments, the solid dosage form comprises three spherical tablets in a single capsule, wherein each spherical tablet comprises a dose of L-theanine, saffron, ashwagandha, wherein a first spherical tablet is formulated for immediate release and comprises a dose of about 60 mg to about 100 mg L-theanine (e.g., about 67 mg L-theanine), a dose of about 2 mg to about 10 mg saffron (e.g., about 5 mg saffron), and a dose of about 20 mg to about 40 mg ashwagandha (e.g., about 27 mg ashwagandha), wherein a second spherical tablet is formulated for extended release and comprises a dose of about 60 mg to about 100 mg L-theanine (e.g., about 67 mg L-theanine), a dose of about 8 mg to about 12 mg saffron (e.g., about 10 mg saffron), and a dose of about 20 mg to about 40 mg ashwagandha (e.g., about 27 mg ashwagandha), wherein a third spherical tablet is formulated for extended release and comprises a dose of about 60 mg to about 100 mg L-theanine (e.g., about 67 mg L-theanine), a dose of about 10 mg to about 20 mg saffron (e.g., about 15 mg saffron), and a dose of about 20 mg to about 40 mg ashwagandha (e.g., about 27 mg ashwagandha), wherein the second spherical tablet and the third spherical tablet each comprise a rate-controlling polymer.

In some embodiments, the solid dosage form comprises three spherical tablets in a single capsule, wherein each spherical tablet comprises a dose of L-theanine, saffron, and ashwagandha, wherein a first spherical tablet is formulated for immediate release and comprises a dose of about 67 mg L-theanine, a dose of about 4 mg saffron, and a dose of about 27 mg ashwagandha, wherein a second spherical tablet is formulated for extended release and comprises a dose of about 67 mg L-theanine, a dose of about 9 mg saffron, and a dose of about 27 mg ashwagandha, wherein a third spherical tablet is formulated for extended release and comprises a dose of about 67 mg L-theanine, a dose of about 15 mg saffron, and a dose of about 27 mg ashwagandha, wherein the second spherical tablet and the third spherical tablet each comprise a rate-controlling polymer.

It should be understood that if the solid dosage form comprises, for example, a dose of 66 mg of L-theanine, a dose of 15 mg saffron, and/or a dose of 27 mg ashwagandha, the amount of L-theanine, saffron, and/or ashwagandha present in the solid dosage form may exceed 66 mg, 15 mg, and 27 mg respectively to account for e.g., loss during manufacturing. In some embodiments, the spherical tablet comprises an amount of L-theanine that is greater than the dose of L-theanine. In some embodiments, the spherical tablet comprises an amount of L-theanine that is about 1.1-fold, about 1.2-fold, about 1.3-fold, about 1.4-fold, or about 1.5-fold greater than the dose of L-theanine. In some embodiments, the spherical tablet comprises an amount of saffron that is greater than the dose of saffron. In some embodiments, the spherical tablet comprises an amount of saffron that is about 1.1-fold, about 1.2-fold, about 1.3-fold, about 1.4-fold, or about 1.5-fold greater than the dose of saffron. In some embodiments, the spherical tablet comprises an amount of ashwagandha that is greater than the dose of ashwagandha. In some embodiments, the spherical tablet comprises an amount of ashwagandha that is about 1.1-fold, about 1.2-fold, about 1.3-fold, about 1.4-fold, or about 1.5-fold greater than the dose of ashwagandha.

In some embodiments, the spherical tablet formulated for extended release releases the dose of L-theanine, saffron, and/or ashwagandha in about 1 hour to about 12 hours under gastrointestinal dissolution conditions. In some embodiments, the spherical tablet formulated for extended release releases the dose of L-theanine, saffron, and/or ashwagandha in about 3 hours to about 12 hours under gastrointestinal dissolution conditions. In some embodiments, the spherical tablet formulated for extended release releases the dose of L-theanine, saffron, and/or ashwagandha in about 3 hours to about 9 hours under gastrointestinal dissolution conditions. In some embodiments, the spherical tablet formulated for extended release releases the dose of L-theanine, saffron, and/or ashwagandha in about 5 hours to about 10 hours under gastrointestinal dissolution conditions. In some embodiments, the spherical tablet formulated for extended release releases the dose of L-theanine, saffron, and/or ashwagandha in about 6 hours to about 9 hours under gastrointestinal dissolution conditions. In some embodiments, the spherical tablet formulated for extended release releases the dose of L-theanine, saffron, and/or ashwagandha in about 6 hours, about 7 hours, about 8 hours, or about 9 hours under gastrointestinal dissolution conditions.

In some embodiments, the spherical tablet formulated for immediate release releases the dose of L-theanine, saffron, and/or ashwagandha in less than about 2 hours under gastrointestinal dissolution conditions. In some embodiments, the spherical tablet formulated for immediate release releases the dose of L-theanine, saffron, and/or ashwagandha in about 30 minutes to about 90 minutes under gastrointestinal dissolution conditions. In some embodiments, the spherical tablet formulated for immediate release releases the dose of L-theanine, saffron, and/or ashwagandha in about 5 minutes, about 10 minutes, about 15 minutes, about 20 minutes, about 25 minutes, about 30 minutes, about 35 minutes, about 40 minutes, about 45 minutes, about 50 minutes, about 55 minutes about 60 minutes, about 65 minutes, about 70 minutes, about 75 minutes, about 80 minutes, about 85 minutes, or about 90 minutes under gastrointestinal dissolution conditions.

In some embodiments, the spherical tablet formulated for extended release comprises a rate controlling polymer in an amount. In some embodiments, the amount of the rate controlling polymer of the tablet formulated for extended release is about 5% to about 15% by weight of the tablet. In some embodiments, the amount is about 5%, about 6%, about 7%, about 8%, about 9%, about 10%, about 11%, about 12%, about 13%, about 14%, or about 15% by weight of the tablet. In some embodiments, the amount of the rate controlling polymer of the tablet formulated for extended release is about 15% to about 25% by weight of the tablet. In some embodiments, the amount is about 15%, about 16%, about 17%, about 18%, about 19%, about 20%, about 21%, about 22%, about 23%, about 24%, or about 25% by weight of the tablet. In some embodiments, the spherical tablet formulated for extended release comprises a coating, wherein the coating comprises a rate controlling polymer in an amount. In some embodiments, the amount of the rate controlling polymer of the coating formulated for extended release is about 0.5% to about 5% by weight of the tablet. In some embodiments, the amount is about 1%, about 2%, about 3%, about 4%, or about 5% by weight of the tablet. In some embodiments, the rate controlling polymer comprises HPMC, HPC, MCC, and a combination thereof. In some embodiments, the rate controlling polymer comprises HPMC, HPC, and MCC. In some embodiments, the HPMC is a grade E15 HPMC, a grade K4M HPMC, or a combination thereof.

In some embodiments, the spherical tablet formulated for immediate release comprises a disintegrant in an amount. In some embodiments, the amount of the disintegrant of the tablet formulated for immediate release is about 0.5% to about 12% by weight of the tablet. In some embodiments, the amount of the disintegrant of the tablet formulated for immediate release is about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 10.5%, about 11%, about 11.5%, or about 12% by weight of the tablet. In some embodiments, the disintegrant comprises psyllium husk, oat fiber, or a combination thereof.

In some embodiments, at least one spherical tablet of the solid dosage form comprises a colorant. In some embodiments, at least one spherical tablet of the solid dosage form does not comprise a colorant. In some embodiments, the spherical tablets of the solid dosage form are substantially similar in color. In some embodiments, the spherical tablets of the solid dosage form are substantially different in color. In some embodiments, saffron present in at least one spherical tablet of the solid dosage form yields a color. In some embodiments, at least two spherical tablets of the solid dosage form comprise a different dose of saffron, wherein the different dose yields a difference in color between the at least two spherical tablets. In some embodiments, at least one spherical tablet is a brown in color due to the presence of saffron and ashwagandha. In some embodiments, at least two spherical tablets of the solid dosage form comprise a dose of saffron and/or ashwagandha, wherein the dose is different, and wherein the spherical tablet comprising an increased dose of saffron and/or ashwagandha is darker in color compared to the spherical tablet with a lower dose of saffron.

In some embodiments, the solid dosage form comprises three spherical tablets in a single capsule, wherein each spherical tablet comprises a dose of L-theanine, saffron, and ashwagandha, wherein the spherical tablets comprise an immediate release spherical tablet, an intermediate release spherical tablet, and an extended release spherical tablet; wherein the immediate release spherical tablet a dose of about 67 mg L-theanine, a dose of about 4 mg saffron, and a dose of about 27 mg ashwagandha, the intermediate release spherical tablet comprises a dose of about 67 mg L-theanine, a dose of about 9 mg saffron, and a dose of about 27 mg ashwagandha, and the extended release spherical tablet comprising a dose of about 67 mg L-theanine, a dose of about 15 mg saffron, and a dose of about 27 mg ashwagandha, wherein the immediate release spherical tablet is formulated to release at least about 85% of the dose contained therein in less than about 2 hours, the intermediate release spherical tablet is formulated to release at least about 85% of the dose contained therein in about 2 hours to about 6 hours (e.g., about 4 hours), and the extended release spherical tablet is formulated to release at least about 85% of the dose contained therein in about in about 4 hours to about 12 hours (e.g., about 8 hours), each as measured upon exposure to gastrointestinal dissolution conditions or following oral administration.

In some embodiments, the intermediate release spherical tablet and the extended release spherical tablet each comprise a rate-controlling polymer. In some embodiments, the intermediate release spherical tablet comprises an amount of the rate controlling polymer that is about 5% to about 15% by weight of the tablet (e.g., about 8% to about 12% by weight of the tablet). In some embodiments, the extended release spherical tablet comprises an amount of the rate controlling polymer that is about 15% to about 25% by weight of the tablet (e.g., about 15% to about 20% by weight of the tablet). In some embodiments, the intermediate release spherical tablet and the extended release spherical tablet each comprise HPMC. In some embodiments, the intermediate release spherical tablet comprises an amount of the HPMC that is about 5% to about 15% by weight of the tablet (e.g., about 8% to about 12% by weight of the tablet) and the extended release spherical tablet comprises an amount of the HPMC that is about 15% to about 25% by weight of the tablet (e.g., about 15% to about 20% by weight of the tablet). In some embodiments, the intermediate release spherical tablet and the extended release spherical tablet each comprise an about 2:1 to about 1:2 ratio (e.g., an about 1:1 ratio) of grade E15 HPMC and a grade K4M HPMC. In some embodiments, the intermediate release spherical tablet comprises an amount of grade E15 HPMC that is about 2% to about 10% by weight of the tablet (e.g., about 4% to about 6% by weight of the tablet) and an amount of grade K4M HPMC that is about 2% to about 10% by weight of the tablet (e.g., about 4% to about 6% by weight of the tablet), wherein the ratio of grade E15 HPMC to grade K4M HPMC is about 2:1 to about 1:2 (e.g., about 1:1); and the extended release tablet comprises an amount of grade E15 HPMC that is about 5% to about 12% by weight of the tablet (e.g., about 6% to about 9% by weight of the tablet) and an amount of grade K4M HPMC that is about 5% to about 12% by weight of the tablet (e.g., about 6% to about 9% by weight of the tablet), wherein the ratio of grade E15 HPMC to grade K4M HPMC is about 2:1 to about 1:2 (e.g., about 1:1). In some embodiments, the immediate release spherical tablet, the intermediate release spherical tablet, and the extended release spherical tablet are different colors, wherein the extended release spherical tablet is darker in color than the intermediate release spherical tablet, and wherein the intermediate release spherical tablet is darker in color than the immediate release spherical tablet.

In some embodiments, the capsule of any of the foregoing solid dosage forms is formulated for instant release. In some embodiments, the capsule is formulated for delayed release. In some embodiments, the capsule is translucent. In some embodiments, the capsule comprises a rate controlling polymer and gellan gum. In some embodiments, the capsule comprises HPMC and gellan gum. In some embodiments, the capsule lacks an animal-derived ingredient. In some embodiments, the capsule is standard size 0. In some embodiments, the capsule is standard size 00. In some embodiments, the capsule is elongated size 0. In some embodiments, the capsule is elongated size 00. In some embodiments, the capsule is standard size 1. In some embodiments, the capsule is standard size 2.

In some embodiments, the spherical tablets of the foregoing dosage forms are substantially similar in size and/or shape. In some embodiments, the spherical tablets are a modified ball shape. In some embodiments, the modified ball shape comprises a diameter of about 6 mm to about 10 mm. In some embodiments, the modified ball shape comprises a diameter of about 6 mm to about 8 mm. In some embodiments, the modified ball shape comprises a diameter of about 6 mm to about 7 mm. In some embodiments, the modified ball shape comprises a diameter of about 6 mm, about 6.4 mm, about 6.5 mm, about 6.7 mm, about 6.9 mm, about 7 mm, or about 7.5 mm. In some embodiments, the modified ball shape comprises a diameter of about 6.4 mm. In some embodiments, the modified ball shape comprises a diameter of about 6.7 mm. In some embodiments, the modified ball shape comprises a diameter of about 6.9 mm. In some embodiments, the spherical tablet comprises a diameter of about 6 mm, about 6.4 mm, about 6.7 mm, or about 6.9 mm and the capsule is standard size 00.

In some embodiments, the capsule of any of the foregoing solid dosage forms is substantially filled by the spherical tablets. In some embodiments, the spherical tablets comprise a diameter and the capsule comprises a circular cross-section transverse to a longest dimension, wherein the diameter of the spherical tablets is about 90% to about 99% of the diameter of the circular cross-section. In some embodiments, the spherical tablets of the solid dosage form comprise a combined volume, wherein the combined volume is at least about 90% to about 99% of the volume of the capsule.

### (3) Formulations for Supplementing Dietary Iron

In some embodiments, the disclosure provides a solid dosage form formulated to supplement dietary iron. In some embodiments, the solid dosage form comprises a plurality of spherical tablets (e.g., 2, 3, 4, or more spherical tablets) in a capsule, wherein each spherical tablet of the plurality comprises a dose of iron. In some embodiments, the solid dosage form is formulated to provide release of iron in a manner that promotes absorption following consumption without inducing substantial gastrointestinal discomfort. In some embodiments, at least one spherical tablet of the plurality further comprises a dose of vitamin C, vitamin A, or both. Without being bound by theory, inclusion of vitamin C and/or vitamin A provides the benefit(s) of antioxidant support and/or promoting iron absorption following consumption. In some embodiments, the solid dosage form is formulated for consumption in a dosing regimen comprising one capsule taken once per day every 1 day, 2 days, or 3 days. In some embodiments, the solid dosage form is formulated for consumption in a dosing regimen comprising one capsule taken once per day every 2 days. In some embodiments, the solid dosage form is formulated for consumption in the morning.

Iron absorption from the GI tract is regulated, at least in part, by the hormone hepcidin. Hepcidin functions to inhibit iron transport from the small intestine to the bloodstream (see, e.g., Collins, et al (2008) J Nutr 138:2284). Endogenous hepcidin levels are lowest in the morning and peak around midday (e.g., about 3 PM to about 5 PM; see, e.g., Moretti et al (2015) Blood 126:1981; Kemna, et al (2007) Clin Chem 53:620). In some embodiments, the solid dosage form is formulated for customized release of iron following consumption that promotes gastrointestinal absorption based upon a circadian rhythm of hepcidin.

In some embodiments, the disclosure provides a solid dosage form comprising (i) at least one immediate release spherical tablet comprising a dose of iron, (ii) at least one intermediate release spherical tablet comprising a dose of iron, and (iii) at least one extended release spherical tablet comprising a dose of iron. In some embodiments, the solid dosage form comprises three spherical tablets, wherein the three spherical tablets comprise (i) an immediate release spherical tablet comprising a dose of iron, (ii) an intermediate release spherical tablet comprising a dose of iron, and (iii) an extended release spherical tablet comprising a dose of iron. In some embodiments, the immediate release spherical tablet is formulated to release at least about 85% of the dose of iron contained therein in less than about 2 hours (e.g., less than about 1 hour or less than about 0.5 hours), as measured upon exposure to gastrointestinal dissolution conditions or following oral administration. In some embodiments, the intermediate release spherical tablet is formulated to release at least about 85% of the dose of iron contained therein in about 3 hours to about 6 hours (e.g., about 4 hours), as measured upon exposure to gastrointestinal dissolution conditions or following oral administration. In some embodiments, the extended release spherical tablet is formulated to release at least about 85% of the dose of iron contained therein in about 4 hours to about 8 hours (e.g., about 6 hours), as measured upon exposure to gastrointestinal dissolution conditions or following oral administration.

Conventional iron supplements are characterized by providing release of large quantities of iron in the GI tract following consumption, which in turn results in poor iron absorption and presence of unabsorbed iron in the GI tract that is associated with GI discomfort, constipation, and bloating (see, e.g., Bloor, et al (2021) Microbiol Res 12:491). Without being bound by theory, consumption of the solid dosage form provides release of iron in a manner that promotes absorption and prevents gastrointestinal discomfort associated with iron retention in the GI tract. For example, in some embodiments, following consumption of the solid dosage form in the morning, the immediate release spherical tablet releases the dose of iron contained therein when endogenous hepcidin levels are lowest, the intermediate release spherical tablet releases the dose of iron contained therein over a duration of about 2-6 hours (e.g., about 4 hours) prior to peaking of endogenous hepcidin levels (thereby promoting GI absorption of iron without substantial GI discomfort due to iron retention in the GI tract), and the extended release spherical tablet release the dose of iron contained therein over a duration of about 4-8 hours (thereby promoting GI comfort throughout the day following the consumption).

In some embodiments, the solid dosage form comprises a plurality of spherical tablets (e.g., 2, 3, 4, or more spherical tablets) in a single capsule, wherein at least one spherical tablet of the plurality comprises a dose of iron (e.g., about 0.9 mg to about 65 mg). In some embodiments, the solid dosage form comprises three spherical tablets in a single capsule, wherein at least one of the spherical tablets comprises a dose of iron (e.g., about 0.9 mg to about 65 mg). In some embodiments, the solid dosage form comprises three spherical tablets in a single capsule, wherein each of the spherical tablets comprises a dose of iron (e.g., about 0.9 mg to about 65 mg).

In some embodiments, the solid dosage form comprises a plurality of spherical tablets (e.g., 2, 3, 4, or more spherical tablets) in a single capsule, wherein each spherical tablet of the plurality comprises a dose of iron (e.g., about 0.9 mg to about 65 mg), and wherein at least one spherical tablet of the plurality is formulated for immediate release and at least one spherical tablet of the plurality is formulated for extended release, wherein the at least one spherical tablet formulated for extended release comprises a rate-controlling polymer. In some embodiments, the solid dosage form comprises a plurality of spherical tablets (e.g., 2, 3, 4, or more spherical tablets) in a single capsule, wherein each spherical tablet of the plurality comprises a dose of iron (e.g., about 0.9 mg to about 65 mg), and wherein at least one spherical tablet of the plurality is formulated for immediate release, at least one spherical tablet of the plurality is formulated for intermediate release, and at least one spherical tablet of the plurality is formulated for extended release, wherein the at least one spherical tablet formulated for intermediate release and the at least one spherical tablet formulated for extended release each comprise a rate-controlling polymer. In some embodiments, the solid dosage form comprises three spherical tablets (e.g., 2, 3, 4, or more spherical tablets) in a single capsule, wherein each spherical tablet comprises a dose of iron (e.g., about 0.9 mg to about 65 mg), and wherein at least one spherical tablet is formulated for immediate release, and wherein at least one spherical tablet of the plurality is formulated for extended release, said spherical tablet formulated for extended release comprising a rate-controlling polymer. In some embodiments, the solid dosage form comprises three spherical tablets (e.g., 2, 3, 4, or more spherical tablets) in a single capsule, wherein each spherical tablet comprises a dose of iron (e.g., about 0.9 mg to about 65 mg), wherein a first spherical tablet is formulated for immediate release, a second spherical tablet is formulated for extended release, and a third spherical tablet is formulated for extended release, wherein the second spherical tablet and the third spherical tablet each comprise a rate-controlling polymer. In some embodiments, the solid dosage form comprises three spherical tablets (e.g., 2, 3, 4, or more spherical tablets) in a single capsule, wherein each spherical tablet comprises a dose of iron (e.g., about 0.9 mg to about 65 mg), wherein a first spherical tablet is formulated for immediate release, a second spherical tablet is formulated for intermediate release, and a third spherical tablet is formulated for extended release, wherein the second spherical tablet and the third spherical tablet each comprise a rate-controlling polymer.

In some embodiments, the dose of iron is about 0.9 mg to about 65 mg, about 6 mg to about 65 mg, about 8 mg to about 65 mg, about 10 mg to about 65 mg, about 12 mg to about 65 mg, about 14 mg to about 65 mg, about 4 mg to about 50 mg, about 6 mg to about 50 mg, about 8 mg to about 50 mg, about 10 mg to about 50 mg, about 12 mg to about 50 mg, about 14 mg to about 50 mg, about 16 mg to about 50 mg, about 18 mg to about 50 mg, about 4 mg to about 40 mg, about 6 mg to about 40 mg, about 8 mg to about 40 mg, about 10 mg to about 40 mg, about 12 mg to about 40 mg, about 14 mg to about 40 mg, about 16 mg to about 40 mg, about 4 mg to about 20 mg, about 6 mg to about 20 mg, about 8 mg to about 20 mg, about 10 mg to about 20 mg, about 12 mg to about 20 mg, about 14 mg to about 20 mg, about 16 mg to about 20 mg, about 4 mg to about 12 mg, about 6 mg to about 11.2 mg, about 6 mg to about 10.64 mg, about 6 mg to about 10 mg, about 7.2 mg to about 10.64 mg, about 7.2 mg to about 10 mg, about 7.2 mg to about 8.8 mg, about 7 mg to about 9 mg, or about 8 mg, or about 18 mg. In some embodiments, the dose of iron is about 7 mg to about 9 mg iron. In certain embodiments, the dose of iron is about 7.2 mg to about 10.64 mg iron. In other embodiments, the dose of iron is about 7.2 mg to about 10 mg iron. In still other embodiments, the dose of iron is about 7.2 mg to about 8.8 mg iron. In one embodiment, the composition comprises about 8 mg iron. In other embodiments, the dose of iron is about 16 mg to about 27 mg iron. In other embodiments, the dose of iron is about 16 mg to about 23 mg iron. In still other embodiments, the dose of iron is about 16 mg to about 22 mg iron. In other embodiments, the dose of iron is about 16 mg to about 20 mg iron. In one embodiment, the composition comprises about 18 mg iron. In one embodiment, the composition comprises about 20 mg iron. In certain embodiments, the iron is present in the form of an iron salt. In other embodiments, the iron is in the form of ferrous bisglycinate. It should be understood that if the solid dosage form comprises, for example, a dose of 8 mg of iron, and the iron is present in the form of a chemical compound comprising iron, the dosage form may comprise greater than 8 mg of the iron compound such that the total dose of iron present in the solid dosage form is 8 mg. Further, it should be understood that if the solid dosage form comprises, for example, a dose of 8 mg of iron, the amount of iron present in the solid dosage form may exceed 8 mg to account for e.g., loss during manufacturing.

In some embodiments, at least one spherical tablet of the solid dosage form further comprises a dose of vitamin A, a dose of vitamin C, and/or a dose of probiotics. In some embodiments, at least one spherical tablet of the solid dosage form further comprises a dose of vitamin A (e.g., a dose of a provitamin A, e.g., a dose of beta-carotene) and/or a dose of vitamin C (e.g., a dose of ascorbic acid). In some embodiments, at least one spherical tablet of the solid dosage form further comprises a dose of vitamin A (e.g., a dose of a provitamin A, e.g., a dose of beta-carotene), but not a dose of vitamin C. In some embodiments, at least one spherical tablet of the solid dosage form further comprises a dose of vitamin C (e.g., a dose of ascorbic acid), but not a dose of vitamin A. In some embodiments, each of the spherical tablets of the solid dosage form further comprise a dose of vitamin A, a dose of vitamin C, and/or a dose of probiotics. In some embodiments, at least one spherical tablet of the solid dosage form further comprises a dose of vitamin A, a dose of vitamin C, and a dose of probiotics. In some embodiments, each of the spherical tablets of the solid dosage form further comprise a dose of vitamin A, a dose of vitamin C, and a dose of probiotics.

In some embodiments, the dose of vitamin A is about 45 μg to about 6 mg, about 45 μg to about 5 mg, about 45 μg to about 4 mg, about 45 μg to about 3 mg, about 45 μg to about 2 mg, about 45 μg to about 1 mg, about 45 μg to about 0.5 mg, about 45 μg to about 0.25 mg, about 45 μg to about 0.1 mg, about 100 μg to about 6 mg, about 100 μg to about 5 mg, about 100 μg to about 4 mg, about 100 μg to about 3 mg, about 100 μg to about 2 mg, about 100 μg to about 1 mg, about 100 μg to about 0.5 mg, about 100 μg to about 0.25 mg, about 500 μg to about 6 mg, about 500 μg to about 5 mg, about 500 μg to about 4 mg, about 500 μg to about 3 mg, about 500 μg to about 2 mg, or about 500 μg to about 1 mg. In some embodiments, the dose is in RAE. In some embodiments, the vitamin A is present as a pro-vitamin A (e.g., a carotenoid). In some embodiments, the vitamin A is present as beta-carotene. In some embodiments, the pro-vitamin A is beta-carotene.

In some embodiments, the dose of vitamin C is about 4.5 mg to about 2 g, about 4.5 mg to about 1.5 g, about 4.5 mg to about 1 g, about 4.5 mg to about 0.5 g, about 4.5 mg to about 250 mg, about 4.5 mg to about 100 mg, about 4.5 mg to about 50 mg, about 100 mg to about 2 g, about 100 mg to about 1.5 g, about 100 mg to about 1 g, about 100 mg to about 0.5 g, or about 100 mg to about 250 mg. In some embodiments, the vitamin C is present as ascorbic acid.

In some embodiments, the dose of probiotics is about 0.5 billion CFU to about 50 billion CFU, about 0.5 billion CFU to about 40 billion CFU, about 0.5 billion CFU to about 30 billion CFU, about 0.5 billion CFU to about 20 billion CFU, about 0.5 billion CFU to about 10 billion CFU, about 0.5 billion CFU to about 5 billion CFU, about 0.5 billion CFU to about 3 billion CFU, about 0.5 billion CFU to about 1 billion CFU, 10 billion CFU to about 50 billion CFU, about 10 billion CFU to about 40 billion CFU, about 10 billion CFU to about 30 billion CFU, or about 10 billion CFU to about 20 billion CFU. In some embodiments, the probiotic comprises a microorganism of a genus selected from Lactobacillus. In some embodiments, the probiotic comprises a Lactobacillus species, a Lactobacillus strains, or a combination thereof. In some embodiments, the probiotic comprises Lactiplantibacillus plantarum (strain designation 299v).

In some embodiments, at least one spherical tablet of the solid dosage form comprises a colorant. In some embodiments, at least one spherical tablet of the solid dosage form does not comprise a colorant. In some embodiments, the spherical tablets of the solid dosage form are substantially similar in color. In some embodiments, the spherical tablets of the solid dosage form are substantially different in color. In some embodiments, the capsule is translucent. In some embodiments, the capsule is semi-opaque. In some embodiments, the capsule is opaque. In some embodiments, the capsule comprises a colorant. In some embodiments, the capsule lacks a colorant.

In some embodiments, the intermediate release spherical tablet and the extended release spherical tablet each comprise a matrix comprising a rate controlling polymer In some embodiments, the intermediate release spherical tablet comprises a matrix comprising a rate controlling polymer in an amount of about 1% to about 15% by weight of the tablet. In some embodiments, the extended release spherical tablet comprises a matrix comprising a rate controlling polymer in an amount of about 2% to about 25% by weight of the tablet. In some embodiments, the intermediate release spherical tablet and the extended release spherical tablet each comprise a coating comprising a rate controlling polymer. In some embodiments, the rate controlling polymer present in the coating and/or the matrix comprise HPMC, HPC, MCC, or a combination thereof. In some embodiments, the rate controlling polymer comprises HPMC, HPC, and MCC. In some embodiments, the HPMC is a grade E15 HPMC, a grade K4M HPMC, or a combination thereof.

In some embodiments, the spherical tablet formulated for immediate release comprises a disintegrant. In some embodiments, the amount of the disintegrant of the tablet formulated for immediate release is about 0.5% to about 12% by weight of the tablet. In some embodiments, the amount of the disintegrant of the tablet formulated for immediate release is about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 10.5%, about 11%, about 11.5%, or about 12% by weight of the tablet. In some embodiments, the disintegrant comprises psyllium husk, oat fiber, or a combination thereof.

In some embodiments, the solid dosage form comprises three spherical tablets in a single capsule, wherein each spherical tablet comprises a dose of iron, wherein the dose of iron is about 1 mg to about 50 mg (e.g., about 20 mg), wherein the spherical tablets comprise an immediate release spherical tablet, an intermediate release spherical tablet, and an extended release spherical tablet, wherein the immediate release spherical tablet is formulated to release at least about 85% of the dose of iron contained therein in less than about 2 hours, the intermediate release spherical tablet is formulated to release at least about 85% of the dose of iron contained therein in about 2 hours to about 6 hours (e.g., about 4 hours), and the extended release spherical tablet is formulated to release at least about 85% of the dose of iron contained therein in about in about 4 hours to about 8 hours (e.g., about 6 hours), each as measured upon exposure to gastrointestinal dissolution conditions or following oral administration. In some embodiments, the immediate release tablet, the intermediate release tablet, and the extended release tablet each further comprise a dose of vitamin C (e.g., a dose of about 5 mg to about 20 mg vitamin C) and a dose of vitamin A (e.g., a dose of about 50 mcg to about 150 mcg RAE vitamin A). In some embodiments, the immediate release tablet and the intermediate release tablet each further comprise a dose of vitamin C (e.g., a dose of about 5 mg to about 20 mg vitamin C), but not a dose of vitamin A; and the extended release tablet further comprises a dose of vitamin A (e.g., a dose of about 50 mcg to about 150 mcg RAE vitamin A), but not a dose of vitamin C. In some embodiments, the immediate release tablet, the intermediate release tablet, and the extended release tablet each further comprise a dose of vitamin C (e.g., a dose of about 5 mg to about 20 mg vitamin C), but not a dose of vitamin A. In some embodiments, the immediate release spherical tablet, the intermediate release spherical tablet, and the extended release spherical tablet are different colors, wherein the extended release spherical tablet is darker in color than the intermediate release spherical tablet, and wherein the intermediate release spherical tablet is darker in color than the immediate release spherical tablet. In some embodiments, the extended release spherical tablet is lighter in color than the intermediate release spherical tablet, and the intermediate release spherical tablet is lighter in color than the immediate release spherical tablet.

In some embodiments, the capsule of any of the foregoing solid dosage forms is formulated for instant release. In some embodiments, the capsule is formulated for delayed release. In some embodiments, the capsule is translucent. In some embodiments, the capsule comprises a rate controlling polymer and gellan gum. In some embodiments, the capsule comprises HPMC and gellan gum. In some embodiments, the capsule lacks an animal-derived ingredient. In some embodiments, the capsule is standard size 0. In some embodiments, the capsule is standard size 00. In some embodiments, the capsule is elongated size 0. In some embodiments, the capsule is elongated size 00.

In some embodiments, the spherical tablets of the solid dosage form are substantially similar in size and/or shape. In some embodiments, the spherical tablets are a modified ball shape. In some embodiments, the modified ball shape comprises a diameter of about 4 mm to about 10 mm. In some embodiments, the modified ball shape comprises a diameter of about 6 mm to about 10 mm. In some embodiments, the modified ball shape comprises a diameter of about 4 mm to about 8 mm. In some embodiments, the modified ball shape comprises a diameter of about 6 mm to about 8 mm. In some embodiments, the modified ball shape comprises a diameter of about 6 mm to about 7 mm. In some embodiments, the modified ball shape comprises a diameter of about 6 mm, about 6.4 mm, about 6.5 mm, about 6.7 mm, about 6.9 mm, about 7 mm, or about 7.5 mm. In some embodiments, the modified ball shape comprises a diameter of about 6.4 mm. In some embodiments, the modified ball shape comprises a diameter of about 6.7 mm. In some embodiments, the modified ball shape comprises a diameter of about 6.9 mm. In some embodiments, the spherical tablet comprises a diameter of about 4 mm to about 6.2 mm and the capsule is standard size 2. In some embodiments, the spherical tablet comprises a diameter of about 4 mm to about 6.7 mm and the capsule is standard size 1. In some embodiments, the spherical tablet comprises a diameter of about 4 mm to about 7.5 mm and the capsule is standard size 0. In some embodiments, the spherical tablet comprises a diameter of about 6 mm, about 6.4 mm, about 6.7 mm, or about 6.9 mm and the capsule is standard size 00.

In some embodiments, the capsule of the solid dosage form is substantially filled by the spherical tablets. In some embodiments, the spherical tablets comprise a diameter and the capsule comprises a circular cross-section transverse to a longest dimension, wherein the diameter of the spherical tablets is about 90% to about 99% of the diameter of the circular cross-section. In some embodiments, the spherical tablets of the solid dosage form comprise a combined volume, wherein the combined volume is at least about 90% to about 99% of the volume of the capsule.

### Methods of Producing

In some embodiments, the solid dosage forms described herein are produced using any suitable methods known in the art.

In some embodiments, a tablet described herein is produced using conventional tableting methods known in the art. Conventional methods for preparing solid dosage forms include, but are not limited to, dry mixing, direct compression, milling, dry or non-aqueous granulation, wet granulation, or fusion. Lachman et al., The Theory and Practice of Industrial Pharmacy (1986).

In some embodiments, the method comprises direct compression. In some embodiments, a tablet is produced by compressing, in a suitable machine, a mixture of an active ingredient described herein (e.g., 1, 2, 3, 4 or more active ingredients described herein) into a solid base forms. In some embodiments, the active ingredient and the excipient are combined to form a “bulk blend.” In some embodiments, the bulk blend is homogenous, i.e., the active ingredient is dispersed evenly throughout such that the bulk blend may be readily subdivided into equally effective unit dosage forms. In some embodiments, the bulk blend is filled into a dye mold and compressed.

In some embodiments, a plurality of solid base forms are produced at once (e.g., with a dye mold that has multiple mold forms), while in other embodiments, solid base forms are produced one at a time sequentially. In certain embodiments, the excipient and the active ingredient form the mixture that is compressed. In other embodiments, the excipient forms the mixture that is compressed, and the nutrient is applied as a coating. In some embodiments, the coating is applied to the plurality of solid base forms to produce the tablet. This may be done, for example, by rotating the plurality of solid base forms in a drum and spraying with a solution or slurry of an coating described herein, then suspending spraying and rotating the drum to dry the layer. In some embodiments, a plurality of coating layers are applied. In some embodiments, the coating is applied and then dried.

In some embodiments, the tablet is produced using a 3D printing method. Suitable equipment assemblies for 3D printing of a tablet are known in the art (see, e.g., U.S. Pat. Nos. 9,339,489; 9,314,429; 8,888,480; and 8,828,411, each of which are incorporated herein by reference).

In some embodiments, a solid dosage form described herein is produced by filling a capsule with a plurality of tablets. Methods for filling capsules are known in the art.

### Methods of Use

In some embodiments, the disclosure provides a method for increasing bioavailability of an active ingredient (e.g., nutraceutical) described herein in a subject, comprising administering (e.g., orally administering) to the subject a solid dosage form described herein comprising the active ingredient. In some embodiments, the amount of the active ingredient available at the site of action is considered to be equivalent to the amount in systemic circulation following the administration. In some embodiments, oral bioavailability is the fraction of the dose of the active ingredient administered to a subject orally that is in systemic circulation following the administration. Methods to measure bioavailability (e.g., oral bioavailability) are known in the art (see, e.g., Olivares-Morales A, et al. Pharm Res. 2014 31:720-30; Currie G M. Pharmacology, Part 2: Introduction to Pharmacokinetics. J Nucl Med Technol. 2018 September; 46 (3): 221-230; Herkenne C, et al. Pharm Res. 2008 January; 25 (1): 87-103; Chow S C. Wiley Interdiscip Rev Comput Stat. 2014; 6 (4): 304-312). In some embodiments, bioavailability is determined by measuring the proportion of the dose of the active ingredient present at the site of action following administration to a subject. In some embodiments, the measurement comprises obtaining a tissue sample from the subject and quantifying the amount of the active ingredient in the tissue sample. In some embodiments, the measurement comprises obtaining a fluid sample from the subject (e.g., a blood sample, a urine sample) and quantifying the amount of the active ingredient in the fluid sample. In some embodiments, bioavailability is calculated as the fraction of the dose of the active ingredient administered to the subject that reaches the systemic circulation in unchanged form. In some embodiments, this definition assumes that all or most of the dose amount administered to the subject that enters systemic circulation will reach the site of action.

In some embodiments, the bioavailability (e.g., oral bioavailability) of the active ingredient is at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98% or at least about 99% of the bioavailability of the active ingredient when administered systemically, e.g., by intravenous injection or infusion. In some embodiments, the bioavailability (e.g., oral bioavailability) of the active ingredient is substantially equivalent to the bioavailability of the active ingredient when administered systemically, e.g., by intravenous injection or infusion.

In some embodiments, the amount of the active ingredient measured in systemic circulation following administration according to a route described herein (e.g., oral administration) to a subject of a solid dosage form described herein is at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98% or at least about 99% of the total amount of the active ingredient in the composition.

In some embodiments, the disclosure provides a method for increasing Cmax of an active ingredient (e.g., nutraceutical) described herein in a subject, comprising administering (e.g., orally administering) to the subject a solid dosage form of the disclosure comprising the active ingredient. In some embodiments, the Cmax of the active ingredient is increased by at least about 1.5-fold, about 2-fold, about 3-fold, about 4-fold, about 5-fold, about 10-fold, about 15-fold, or about 20-fold compared to the Cmax of a control dosage form (e.g., a conventional solid dosage form comprising the active ingredient). In some embodiments, the Cmax of the active ingredient is substantially comparable to the Cmax of a control dosage form (e.g., a conventional solid dosage form comprising the active ingredient). In some embodiments, the Cmax of the active ingredient is decreased by at least about 1.5-fold, about 2-fold, about 3-fold, about 4-fold, about 5-fold, about 10-fold, about 15-fold, or about 20-fold compared to administration of a control dosage form (e.g., a conventional solid dosage form comprising the active ingredient).

In some embodiments, the disclosure provides a method for increasing peak concentration of an active ingredient (e.g., nutraceutical) described herein in a subject, comprising administering (e.g., orally administering) to the subject a solid dosage form of the disclosure comprising the active ingredient. In some embodiments, the peak concentration of the active ingredient is increased by at least about 1.5-fold, about 2-fold, about 3-fold, about 4-fold, about 5-fold, about 10-fold, about 15-fold, or about 20-fold compared to administering a control dosage form (e.g., a conventional solid dosage form comprising the active ingredient) to the subject. In some embodiments, the peak concentration of the active ingredient is substantially comparable to administering a control dosage form (e.g., a conventional solid dosage form comprising the active ingredient) to the subject. In some embodiments, the peak concentration of the active ingredient is decreased by at least about 1.5-fold, about 2-fold, about 3-fold, about 4-fold, about 5-fold, about 10-fold, about 15-fold, or about 20-fold as compared administering a control dosage form (e.g., a conventional solid dosage form comprising the active ingredient) to the subject.

In some embodiments, the disclosure provides a method for increasing Tmax of an active ingredient (e.g., nutraceutical) described herein in a subject, comprising administering (e.g., orally administering) to the subject a solid dosage form of the disclosure comprising the active ingredient. In some embodiments, the Tmax of the active ingredient is increased by at least about 1.5-fold, about 2-fold, about 3-fold, about 4-fold, about 5-fold, about 10-fold, about 15-fold, or about 20-fold compared to administration of a control dosage form (e.g., a conventional solid dosage form comprising the active ingredient).

In some embodiments, the disclosure provides a method for increasing time to peak concentration of an active ingredient (e.g., nutraceutical) described herein in a subject, comprising administering (e.g., orally administering) to the subject a solid dosage form of the disclosure comprising the active ingredient. In some embodiments, the time to peak concentration of the active ingredient is increased by at least about 1.5-fold, about 2-fold, about 3-fold, about 4-fold, about 5-fold, about 10-fold, about 15-fold, or about 20-fold as compared to administering a control dosage form (e.g., a conventional solid dosage form comprising the active ingredient) to the subject.

In some embodiments, the disclosure provides a method for increasing an average AUC of an active ingredient (e.g., nutraceutical) described herein in a subject, comprising administering (e.g., orally administering) to the subject a solid dosage form of the disclosure comprising the active ingredient. In some embodiments, administration of a solid dosage form of the disclosure comprising the active ingredient according to a route of administration described herein (e.g., oral administration) results in an average AUC of the active ingredient that is increased compared to the average AUC following systemic administration (e.g., via intravenous injection) of the composition or administration via the same route of a dosage form comprising a control composition (e.g., a conventional solid dosage form comprising the active ingredient).

In some embodiments, the AUC of a solid dosage form of the disclosure following administration according to a route of administration described herein (e.g., oral administration) is at least about 1.5-fold, about 2-fold, about 3-fold, about 4-fold, about 5-fold, about 10-fold, about 15-fold, or about 20-fold of the AUC of the active ingredient following systemic administration (e.g., via intravenous injection). In some embodiments, the AUC of a solid dosage form of the disclosure following administration according to a route of administration described herein (e.g., oral administration) is at least about 1.5-fold, about 2-fold, about 3-fold, about 4-fold, about 5-fold, about 10-fold, about 15-fold, or about 20-fold of the AUC of the composition following administration via the same route of a control dosage form (e.g., a conventional solid dosage form comprising the active ingredient).

In some embodiments, the disclosure provides a method for increasing percent bioavailability of an active ingredient (e.g., nutraceutical) described herein in a subject, comprising administering (e.g., orally administering) to the subject a solid dosage form of the disclosure comprising the active ingredient. In some embodiments, the percent bioavailability of the active ingredient is increased by at least about 1.5-fold, about 2-fold, about 3-fold, about 4-fold, about 5-fold, about 10-fold, about 15-fold, or about 20-fold compared to percent bioavailability of the active ingredient administered in a control dosage form (e.g., a conventional solid dosage form comprising the active ingredient).

In some embodiments, the disclosure provides a method for reducing a side effect associated with a nutraceutical in a subject, comprising administering to the subject a solid dosage form described herein, wherein the solid dosage form comprises a plurality of tablets in a single capsule, and wherein at least one tablet of the plurality comprises a dose of the nutraceutical. In some embodiments, at least one tablet of the plurality is formulated for immediate release and comprises a dose of the nutraceutical. In some embodiments, at least one tablet of the plurality is formulated for extended release and comprises a dose of the nutraceutical. In some embodiments, the administering results in a bioavailability of the nutraceutical that provides a desired health benefit, while maintaining a Cmax below a threshold associated with the side effect. In some embodiments, the side effect is reduced as compared to administering a control dosage form comprising the dose of the nutraceutical.

In some embodiments, the control dosage form comprises a formulation of the active ingredient, wherein the formulation is characterized by a different release profile than the solid dosage form. In some embodiments, the control dosage form comprises a formulation characterized by the same release profile as the solid dosage form, wherein the formulation comprises an active ingredient that is different than the active ingredient of the solid dosage form. In some embodiments, the control dosage form comprises an instant release formulation of the active ingredient. In some embodiments, the control dosage form comprises an extended release formulation of the active ingredient.

In some embodiments, the disclosure provides a method for customized release of a nutraceutical in a subject, comprising administering to the subject a solid dosage form described herein comprising the nutraceutical. In some embodiments, the customized release of the nutraceutical mimics an endogenous hormone release in the subject. In some embodiments, the customized release of the nutraceutical mimics an endogenous circadian rhythm. In some embodiments, the customized release of the nutraceutical promotes efficient cellular uptake of the nutraceutical following the administering. In some embodiments, the nutraceutical is released at a rate that promotes efficient cellular uptake of the nutraceutical following the administering. In some embodiments, the nutraceutical is released at a rate following that promotes efficient gastrointestinal absorption following the administering.

In some embodiments, the nutraceutical is a compound that is naturally produced in a human subject. In some embodiments, the compound is a hormone, a metabolite, a stimulant, a peptide, a protein, a prebiotic, a probiotic, a vitamin, a mineral, or an amino acid. In some embodiments, the nutraceutical is formulated in a solid dosage form of the disclosure to provide a customized release profile that mimics an endogenous level of the compound, e.g., as measured in serum of the subject. In some embodiments, the nutraceutical is formulated in a solid dosage form of the disclosure to provide a customized release profile that mimics an endogenous level of the compound, wherein the endogenous level of the compound fluctuates as part of a circadian rhythm or a physiological process. In some embodiments, administration of the solid dosage form to the subject increases a level of the compound above an endogenous level of the compound, e.g., as measured in serum of the subject. In some embodiments, the nutraceutical is a compound that alters the level of an endogenous compound when administered to a subject. In some embodiments, the nutraceutical is formulated in a solid dosage form of the disclosure to have a customized release profile that alters the level of the endogenous compound in a manner that mimics a circadian rhythm or a physiological process. In some embodiments, the nutraceutical is formulated in a solid dosage form of the disclosure to provide a customized release profile that promotes gastrointestinal absorption following administration, e.g., based upon endogenous levels of a biomarker that regulates gastrointestinal absorption of the nutraceutical.

### Exemplary Methods for Improving Sleep

The present disclosure relates, at least in part, to methods for improving sleep in a subject comprising administering to the subject a solid dosage form described herein (e.g., a solid dosage form described herein comprising a formulation for improving sleep). In some embodiments, the solid dosage form is one described herein comprising a dose of melatonin.

In some embodiments, method comprises administering the solid dosage form to the subject prior to a nocturnal sleep period. In some embodiments, the subject is administered the solid dosage form about 10 minutes, about 20 minutes, about 30 minutes, about 40 minutes, about 50 minutes, about 60 minutes, about 90 minutes, or about 120 minutes prior to a nocturnal sleep period. In some embodiments, the subject is administered the solid dosage form about 60 minutes prior to a nocturnal sleep period.

In some embodiments, the method comprises administering to the subject a solid dosage form described herein comprising a plurality of tablets (e.g., 2, 3, 4, or more spherical tablets) in a single capsule, wherein at least one tablet of the plurality comprises a dose of melatonin. In some embodiments, the method comprises administering to the subject a solid dosage form described herein comprising three spherical tablets in a single capsule, wherein at least one of the spherical tablets comprises a dose of melatonin (e.g., a dose of about 0.5 mg to about 5 mg melatonin). In some embodiments, the method comprises administering to the subject a solid dosage form described herein comprising three spherical tablets in a single capsule, wherein each of the spherical tablets comprises a dose of melatonin (e.g., a dose of about 0.5 mg to about 5 mg melatonin). In some embodiments, the method comprises administering to the subject a solid dosage form described herein, wherein the solid dosage form described herein comprises three spherical tablets in a single capsule, wherein each spherical tablet comprises a dose of melatonin (e.g., a dose of about 0.5 mg to about 5 mg melatonin), and wherein at least one spherical tablet of the plurality is formulated for immediate release, and wherein at least one spherical tablet of the plurality is formulated for extended release. In some embodiments, the method comprises administering to the subject a solid dosage form described herein, wherein the solid dosage form described herein comprises three spherical tablets in a single capsule, wherein each spherical tablet comprises a dose of melatonin (e.g., a dose of about 0.5 mg to about 5 mg melatonin), wherein one spherical tablet of the three spherical tablets is formulated for immediate release and two spherical tablets of the three spherical tablets are formulated for extended release. In some embodiments, the three spherical tablets comprise an immediate release spherical tablet, a first extended release spherical tablet, and a second extended release spherical tablet. In some embodiments, the immediate release spherical tablet comprises a dose of about 0.5 mg to about 2 mg melatonin (e.g., about 1 mg melatonin), the first extended release spherical tablet comprises a dose of about 2 mg to about 4 mg (e.g., about 3 mg melatonin), and the second extended release spherical tablet comprises a dose of about 0.5 mg to about 2 mg melatonin (e.g., about 1 mg melatonin), wherein the immediate release spherical tablet is formulated to release at least about 85% of the dose of melatonin contained therein in less than about 2 hours (e.g., about 1 hour), the first extended release spherical tablet is formulated to release at least about 85% of the dose of melatonin contained therein in about 6 hours to about 8 hours (e.g., about 6 hours), and the second extended release spherical tablet is formulated to release at least about 85% of the dose of melatonin contained therein in about in about 2 hours to about 4 hours (e.g., about 3 hours), each as measured upon exposure to gastrointestinal dissolution conditions or following oral administration.

In some embodiments, the administering results in improved sleep as compared to administering to the subject a control solid dosage form comprising the dose of melatonin (e.g., a conventional solid dosage form comprising the dose of melatonin). In some embodiments, the control solid dosage form is substantially identical (e.g., visually identical) to the solid dosage form comprising the dose of melatonin. In some embodiments, the control solid dosage form is not substantially identical (e.g., visually identical) to the solid dosage form comprising the dose of melatonin. In some embodiments, the administering results in improved sleep as compared to administering to the subject a control solid dosage form, wherein the control solid dosage form is visually identical to the solid dosage form comprising the dose of melatonin, and wherein the control solid dosage form does not contain a dose of melatonin.

In some embodiments, the administering decreases the time to fall to sleep. In some embodiments, the administering increases the total duration of sleep. In some embodiments, the administering reduces the number of nocturnal awakenings (e.g., the number of occurrences of wake after sleep onset). In some embodiments, the administering increases sleep efficiency (i.e., ratio of hours of sleep to hours attempting to sleep). In some embodiments, the administering increases the quality of sleep. Methods to evaluate quality of sleep in a subject are known in the art and include, e.g., conducting a survey of sleep quality (e.g., a restorative sleep questionnaire, a quality of life (WHO-5) questionnaire, a Next Morning Well-Being (REST-Q) survey, a Consensus Sleep Diary-Core (CSD) survey, or a Pittsburgh Sleep Quality Index (PSQI) survey), monitoring a wearable device configured for collecting sleep quality information (e.g., Fitbit).

In some embodiments, the administering reduces the period for sleep latency. In some embodiments, the period for sleep latency is reduced by at least about 10%, about 20%, about 30%, about 40%, about 50%, about 60%, about 70%, about 80%, about 90%, or about 100%, e.g., as compared to not administering a dosage form to the subject or administering a control dosage form (e.g., a control dosage form lacking a dose of melatonin) to the subject. In some embodiments, the period for sleep latency is reduced by about 30% to about 50%. In some embodiments, the period for sleep latency is reduced by about 40% to about 50%. In some embodiments, the period for sleep latency is reduced by at least about 5 minutes, about 10 minutes, about 15 minutes, or about 20 minutes, e.g., as compared to not administering a dosage form to the subject or administering a control dosage form (e.g., a control dosage form lacking a dose of melatonin) to the subject. In some embodiments, the period for sleep latency is reduce by about 5 minutes to about 20 minutes. In some embodiments, the period for sleep latency is reduced by about 12 minutes. In some embodiments, sleep latency is measured by a PSQI survey. In some embodiments, sleep latency is measured by a wearable sleep tracker (e.g., Fitbit).

In some embodiments, the administering is associated with an improved waking up fresh and rested score as measured by a WHO-5 survey. In some embodiments, the waking up fresh and rested score is increased as compared to not administering a dosage form to the subject or administering a control dosage form (e.g., a control dosage form lacking a dose of melatonin) to the subject.

In some embodiments, the administering is not substantially associated with an undesirable side effect. In some embodiments, the side effect is characterized by tiredness, grouchiness, and/or sleepiness upon waking. In some embodiments, the side effect is measured by a REST-Q survey.

In some embodiments, the administering is not substantially associated with a rebound effect following discontinued administration of the solid dosage form. In some embodiments, the rebound effect is selected from increased sleep onset, reduced total sleep time, and increased number of nocturnal awakenings, each as compared to a baseline nocturnal sleep period (e.g., a baseline nocturnal sleep period measured over about 7 consecutive days prior to administering the solid dosage form) and optionally, as measured by a wearable sleep tracker. In some embodiments, the rebound effect is worsening of well-being as compared to a baseline nocturnal sleep period, optionally as measured by a subjective survey, e.g., a REST-Q survey.

In some embodiments, the administering results in enhanced deep sleep during the second sleep cycle of a nocturnal sleep period, e.g., as compared to not administering a dosage form to the subject or administering a control dosage form (e.g., a control dosage form lacking a dose of melatonin) to the subject. In some embodiments, the second sleep cycle occurs about 1.5 hours to about 3 hours following onset of the nocturnal sleep period. In some embodiments, the administering is not associated with increased deep sleep during the final sleep cycle of a nocturnal sleep period, e.g., as compared to not administering a dosage form to the subject or administering a control dosage form (e.g., a control dosage form lacking a dose of melatonin) to the subject. In some embodiments, the final sleep cycle occurs about 5 to about 8 hours following onset of the nocturnal sleep period. In some embodiments, the administering results in increased light sleep at about 6 hours to about 8 hours following onset of the nocturnal sleep period, e.g., as compared to not administering a dosage form to the subject or administering a control dosage form (e.g., a control dosage form lacking a dose of melatonin) to the subject. In some embodiments, the light sleep comprises stage 1 NREM sleep. In some embodiments, the light sleep comprises stage 1 and stage 2 NREM sleep. Without being bound by theory, enhanced deep sleep during a second sleep cycle (about 1.5 hours to about 3 hours from nocturnal sleep onset) and light sleep occurring at the close of sleep (about 6 hours from nocturnal sleep onset to termination of the nocturnal sleep period) promotes increased well-being upon waking (e.g., increased waking feeling fresh and rested).

In some embodiments, the disclosure provides a method for improving sleep in a plurality of subjects, comprising administering to each subject of the plurality a solid dosage form described herein comprising a plurality of tablets in a single capsule, wherein at least one tablet of the plurality comprises a dose of melatonin. In some embodiments, the administering improves sleep in a proportion of subjects of the plurality. In some embodiments, the proportion of subjects in the plurality that experience improved sleep is compared to the proportion of subjects in a control group that experience improved sleep, wherein the subjects of the control group receive a placebo dosage form (e.g., a visually comparable dosage form lacking a dose of melatonin). In some embodiments, the proportion of subjects of the plurality that experience improved sleep following the administration is greater than the proportion of subjects in the control group that experience improved sleep. In some embodiments, at least about 70%, about 75, about 80%, about 85%, about 90%, or about 95% of the subjects experience waking up feeling fresh and rested following the administering, e.g., as measured by WHO-5 survey. In some embodiments, there is no substantial difference in the proportion of the subject that experience negative side effects (e.g., waking feeling tired, grouchy, and/or groggy) following the administering as compared to the control group, e.g., as measured by a REST-Q survey.

In some embodiments, the disclosure provides a method for improving sleep in a subject, comprising administering to the subject a solid dosage form described herein comprising a plurality of tablets in a single capsule, wherein the tablets each comprise a dose of melatonin, and wherein the release of melatonin mimics endogenous melatonin production associated with a circadian rhythm. In the typical circadian rhythm of healthy adults, endogenous melatonin production increases shortly after dark, reaches a peak in the middle of the night, and then declines throughout the rest of the night to reach a low point shortly before an individual awakens (Grivas T B and Savvidou O D. Scoliosis. 2007 2:6). In some embodiments, a first tablet of the plurality comprises a dose of melatonin in an immediate release formulation described herein and at least one second tablet of the plurality comprises a dose of melatonin in an extended release formulation. In some embodiments, the release of melatonin from the first tablet and the at least one second tablet mimics endogenous melatonin production associated with a circadian rhythm.

In some embodiments, the disclosure provides a method of increasing a nocturnal melatonin level in a subject, comprises administering to the subject a solid dosage form described herein (e.g., a solid dosage form described herein comprising a formulation for improving sleep), wherein the administering occurs prior to a nocturnal sleep period. In some embodiments, the administering occurs about 0.5 hours, about 1 hour, about 1.5 hours, or about 2 hours prior to the nocturnal sleep period. In some embodiments, the increased nocturnal melatonin level is associated with substantially no negative side effects (e.g., grogginess, tiredness, and/or grouchiness) upon waking from the nocturnal sleep period, decreased sleep latency, decreased sleep latency, and/or an improved waking up feeling fresh and rested score (e.g., as measured by a WHO-5 survey).

In some embodiments, the melatonin level is measured in a sample obtained from the subject following the administering. In some embodiments, the sample is a blood sample (e.g., a plasma or serum sample).

In some embodiments, the melatonin level is increased within about 10 minutes, about 15 minutes, about 20 minutes, about 30 minutes, about 40 minutes, about 50 minutes or about 60 minutes of the administering. In some embodiments, the increased melatonin level is maintained for up to about 5 hours, about 5.5 hours, about 6 hours, about 6.5 hours, about 7 hours, about 7.5 hours, or about 8 hours following the administering.

In some embodiments, the method provides a melatonin level characterized by an increased AUC over the nocturnal sleep period, e.g., as compared to a melatonin level measured in a subject not administered the dosage form or administered a control dosage form (e.g., a control dosage form lacking a dose of melatonin). In some embodiments, the AUC is increased by at least about 2-fold, about 3-fold, about 4-fold, about 5-fold, about 10-fold, about 15-fold, about 20-fold, about 25-fold, or about 30-fold over the nocturnal sleep period, e.g., as compared to the melatonin level measured in a subject not administered the dosage form or administered a control dosage form. In some embodiments, the AUC is increased by about 20-fold to about 30-fold over the nocturnal sleep period, e.g., as compared to the melatonin level measured in a subject not administered the dosage form or administered a control dosage form. In some embodiments, the nocturnal sleep period has a duration of about 6 hours, about 7 hours, about 8 hours, or about 9 hours.

In some embodiments, the method provides a melatonin level characterized by an increased peak concentration during the nocturnal sleep period e.g., as compared to a melatonin level measured in a subject not administered the dosage form or administered a control dosage form (e.g., a control dosage form lacking a dose of melatonin). In some embodiments, the peak concentration is increased by at least about 2-fold, about 3-fold, about 4-fold, about 5-fold, about 10-fold, about 15-fold, or about 20-fold, e.g., as compared to the melatonin level measured in a subject not administered the dosage form or administered a control dosage form. In some embodiments, the peak concentration is increased by about 10-fold to about 20-fold, e.g., as compared to the melatonin level measured in a subject not administered the dosage form or administered a control dosage form.

In some embodiments, the method provides a melatonin level characterized by a decreased time to peak concentration during the nocturnal sleep period e.g., as compared to a melatonin level measured in a subject not administered the dosage form or administered a control dosage form (e.g., a control dosage form lacking a dose of melatonin). In some embodiments, the time to peak concentration occurs about 0.5 hours, about 1 hour, about 1.5 hours, about 2 hours, about 2.5 hours, about 3 hours, about 3.5 hours, or about 4 hours earlier, e.g., as compared to the melatonin level measured in a subject not administered the dosage form or administered a control dosage form.

### Exemplary Methods for Reducing Stress

The present disclosure relates, at least in part, to methods for reducing stress, improving mood, and/or supporting normal cortisol levels in a subject comprising administering to the subject a solid dosage form described herein (e.g., a solid dosage form described herein comprising a formulation for reducing stress). In some embodiments, the solid dosage form is administered to the subject during a period characterized by a peak circadian cortisol levels. In some embodiments, the solid dosage form is administered to the subject in the morning. In some embodiments, the solid dosage form is administered to the subject is a dosing regimen. In some embodiments, the dosing regimen comprises one dosage form consumed once daily (e.g., once daily in the morning).

In some embodiments, the solid dosage form is one described herein comprising a dose of a nutraceutical selected from L-theanine, saffron, ashwagandha, and a combination thereof. In some embodiments, the method comprises administering to the subject a solid dosage form described herein comprising a plurality of spherical tablets (e.g., 2, 3, 4, or more spherical tablets) in a single capsule, wherein at least one spherical tablet of the plurality comprises a dose of a nutraceutical selected from L-theanine, saffron, ashwagandha, and a combination thereof. In some embodiments, the method comprises administering to the subject a solid dosage form described herein comprising a plurality of spherical tablets (e.g., 2, 3, 4, or more spherical tablets) in a single capsule, wherein each spherical tablet of the plurality comprises a dose of a nutraceutical selected from L-theanine, saffron, ashwagandha, and a combination thereof, and wherein at least one spherical tablet of the plurality is formulated for immediate release and at least one spherical tablet of the plurality is formulated for extended release.

In some embodiments, the disclosure provides a method for reducing stress and/or supporting normal cortisol levels in a subject, comprising administering to the subject a solid dosage form described herein comprising a plurality of tablets in a single capsule, wherein at least one tablet of the plurality comprises a dose of L-theanine, saffron, and/or ashwagandha. In some embodiments, the administering results in reduced stress and/or normalized cortisol levels as compared to administering a control solid dosage form comprising the dose of L-theanine, saffron, and/or ashwagandha. In some embodiments, the administering results in reduced stress and/or normalized cortisol levels as compared to administering to the subject a control solid dosage form, wherein the control solid dosage form does not contain a dose of L-theanine, saffron, or ashwagandha. In some embodiments, the control dosage form has an appearance that is the same as or different from the solid dosage form comprising the dose of L-theanine, saffron, and/or ashwagandha. In some embodiments, the control solid dosage form is visually identical to the solid dosage form comprising the dose of L-theanine, saffron, and/or ashwagandha.

In some embodiments, the subject has an abnormal metric associated with high stress, and the administering normalizes the metric. In some embodiments, the abnormal metric comprises elevated cortisol, high heart rate, elevated blood pressure, increased heart rate variability, and poor mental performance and/or accuracy during a stressful event. In some embodiments, stress in the subject is measured using a questionnaire. In some embodiments, the questionnaire is selected from the Perceived Stress Scale (PSS). In some embodiments, the self-perceived stress of the subject is improved following the administering as compared to prior to the administering. In some embodiments, the metric is normalized in the subject following the administration and compared to prior to the administration.

In some embodiments, the disclosure provides a method for reducing stress and/or supporting normal cortisol levels in a plurality of subjects, comprising administering to each subject of the plurality a solid dosage form described herein comprising a plurality of tablets in a single capsule, wherein at least one tablet of the plurality comprises a dose of L-theanine, saffron, and/or ashwagandha. In some embodiments, the subjects of the plurality have an abnormal metric associated with high stress. In some embodiments, the administering normalizes the metric in a proportion of subjects of the plurality. In some embodiments, the proportion of subjects in the plurality that experience a normalized metric is compared to the proportion of subjects in a control group that experience a normalized metric, wherein the subjects of the control group receive a placebo dosage form (e.g., a control dosage form lacking a dose of L-theanine, saffron, or ashwagandha, wherein the control dosage form has an appearance that is the same as or different from the solid dosage form), wherein the subjects of the control group have an abnormal metric associated with stress. In some embodiments, the proportion of subjects of the plurality that experience the normalized metric following the administration is greater than the proportion of subjects in the control group that experience the normalized metric.

In some embodiments, the disclosure provides a method for improving mood in a subject, comprising administering to the subject a solid dosage form described herein comprising a plurality of tablets in a single capsule, wherein at least one tablet of the plurality comprises a dose of L-theanine, saffron, and/or ashwagandha. In some embodiments, mood in the subject is measured using a questionnaire for depression, stress and/or anxiety symptoms. In some embodiments, the questionnaire is selected from the Depression Anxiety and Stress Scale-21 (DASS-21), Perceived Stress Scale (PSS), Hamilton Anxiety Rating Scale (HAM-A), Self-rating Depression Scale (SDS), the State-Trait Anxiety Inventory (STAI), Profile of Mood States (POMS), and The Positive and Negative Affect Schedule (PANAS). In some embodiments, the mood of the subject is improved following the administering as compared to prior to the administering.

In some embodiments, the disclosure provides a method for improving mood in a plurality of subjects, comprising administering to each subject of the plurality a solid dosage form described herein comprising a plurality of tablets in a single capsule, wherein at least one tablet of the plurality comprises a dose of L-theanine, saffron, and/or ashwagandha. In some embodiments, the administering improves mood in a proportion of subjects of the plurality. In some embodiments, the proportion of subjects in the plurality that experience an improved mood is compared to the proportion of subjects in a control group that experience improved mood, wherein the subjects of the control group receive a placebo dosage form (e.g., a control dosage form lacking a dose of L-theanine, saffron, or ashwagandha, wherein the control dosage form has an appearance that is the same as or different from the solid dosage form). In some embodiments, the proportion of subjects of the plurality that experience improved mood following the administration is greater than the proportion of subjects in the control group that experience improved mood.

In some embodiments, the disclosure provides a method for supporting normal cortisol levels in a subject, comprising administering to the subject a formulation for reducing stress described herein, wherein the administering alters endogenous cortisol levels in a manner that mimics endogenous cortisol release associated with a circadian rhythm. In the typical circadian rhythm of healthy adults, cortisol levels begin rising early in the morning, peak shortly after an individual wakes up, decline throughout the rest of the day, and reach a low point (baseline) in the middle of the night (see Jones C and Gwennin C. Physiol Rep. 2021 8 (24): e14644; Russel G and Lightman S. Nat Rev Endocrinol. 2019 15 (9): 525-534). In some embodiments, the administering occurs in the morning. In some embodiments, the administering occurs in a period of the day comprising a peak endogenous cortisol level. In some embodiments, the administering results in a reduction of the endogenous cortisol level. In some embodiments, the administering prevents a rise in an endogenous cortisol level.

### Exemplary Methods for Iron Supplementation

The present disclosure relates, at least in part, to methods for ameliorating or preventing iron deficiency in a subject comprising administering to the subject a solid dosage form described herein (e.g., a solid dosage form described herein comprising a formulation for supplementing dietary iron). In some embodiments, the solid dosage form is one described herein comprising a dose of iron. In some embodiments, the subject is a human. In some embodiments, the subject is a man. In some embodiments, the subject is a woman. In some embodiments, the woman is pregnant. In some embodiments the woman is not pregnant (i.e., non-natal).

In some embodiments, the method comprises administering the dosage form to the subject in a dosing regimen. In some embodiments, the dosing regimen comprises administering a single solid dosage form once every about 1 day, about 2 days, or about 3 days. In some embodiments, the dosing regimen comprises administering a single solid dosage form once every about 2 days. In some embodiments, the solid dosage form is administered in the morning.

In some embodiments, the method comprises administering to the subject a solid dosage form described herein comprising a plurality of tablets (e.g., 2, 3, 4, or more spherical tablets) in a single capsule, wherein at least one tablet of the plurality comprises a dose of iron (e.g., a dose of about 5 mg to about 50 mg iron). In some embodiments, the method comprises administering to the subject a solid dosage form described herein comprising three spherical tablets in a single capsule, wherein at least one of the spherical tablets comprises a dose of iron (e.g., a dose of about 5 mg to about 50 mg iron). In some embodiments, the method comprises administering to the subject a solid dosage form described herein comprising three spherical tablets in a single capsule, wherein each of the spherical tablets comprises a dose of iron (e.g., a dose of about 5 mg to about 50 mg iron). In some embodiments, the method comprises administering to the subject a solid dosage form described herein, wherein the solid dosage form described herein comprises three spherical tablets in a single capsule, wherein each spherical tablet comprises a dose of iron (e.g., a dose of about 5 mg to about 50 mg iron), wherein one spherical tablet of the three spherical tablets is formulated for immediate release, one of the three spherical tablets is formulated for intermediate release, and one of the three spherical tablets is formulated for extended release. In some embodiments, the immediate release spherical tablet, the intermediate release spherical tablet, and the extended release spherical tablet each comprises a dose of about 5 mg to about 50 mg iron (e.g., about 20 mg iron), wherein the immediate release spherical tablet is formulated to release at least about 85% of the dose of iron contained therein in less than about 2 hours, the intermediate release spherical tablet is formulated to release at least about 85% of the dose of iron contained therein in about 3 hours to about 6 hours (e.g., about 4 hours), and the extended release spherical tablet is formulated to release at least about 85% of the dose of iron contained therein in about in about 4 hours to about 8 hours (e.g., about 6 hours), each as measured upon exposure to gastrointestinal dissolution conditions or following oral administration. In some embodiments, at least one spherical tablet further comprises a dose of vitamin C, a dose of vitamin A, or a dose of vitamin C and a dose of vitamin A.

In some embodiments, the administering results in enhanced gastrointestinal absorption of iron as compared to administering to the subject a control solid dosage form comprising the dose of iron (e.g., a conventional solid dosage form comprising the dose of iron). In some embodiments, the administering increases an iron level in the subject. In some embodiments, the administering is not substantially associated with gastrointestinal discomfort.

### Administering and Dose

In some embodiments, a solid dosage form described herein is formulated for administration to a mammal. In some embodiments, the mammal is a human. In some embodiments, the solid dosage form is formulated for administration to a woman, a man, or both. In some embodiments, the solid dosage form is formulated for administration to a child, an adult, or both.

In some embodiments, the solid dosage form is formulated for oral administration.

In some embodiments, the solid dosage form comprises a therapeutically effective dose of an active ingredient described herein.

In some embodiments, the solid dosage form is administered daily. In some embodiments, the solid dosage form is administered more than once per day. In some embodiments, the solid dosage form is administered in the morning. In some embodiments, the solid dosage form is administered in the evening. In some embodiments, the solid dosage form is administered prior to bedtime.

In some embodiments, the solid dosage form is administered on multiple occasions, wherein intervals between single dosage are hourly, daily, weekly, monthly, or yearly. In some embodiments, the solid dosage form is administered for a duration lasting until a disease or disorder in a subject is ameliorated or treated. In some embodiments, the solid dosage form is administered following the duration to prevent recurrence of the disease or disorder.

For any active ingredient described herein, the therapeutically effective amount is estimated initially using activity assays in cell cultures and/or animals. For example, in some embodiments, a dose is formulated in an animal model to achieve a circulating concentration range that achieves an ECso as determined in an in vitro activity assay (e.g., the concentration of test compound, which achieves half-maximal effective activity). Such information is used to determine an effective dose in humans. In some embodiments, the dose is selected to provide a plasma level of the active that is sufficient to achieve a therapeutic effect.

### Kit

In some embodiments, the disclosure provides a kit comprising a plurality of solid dosage forms described herein.

In some embodiments, the plurality of solid dosage forms is packaged with a scented kit. Said scented insert may be, for example, included in a container with the plurality of solid dosage forms described herein. Including a scented insert may associate a desirable odor with the solid dosage forms or reduce the perception of an undesirable odor, which may increase a subject's compliance with a dosing regimen.

In some embodiments, the insert comprises polymer and an odorant agent. The odorant agent may be derived from natural sources. In some embodiments, the odorant agent comprises a plant oil, one or more compounds derived from plants, or an oil comprising one or more compounds derived from a plant. In some embodiments, the odorant agent is a plant oil, one or more compounds derived from plants, or an oil comprising one or more compounds derived from a plant. In some embodiments, the odorant agent comprises an oil, extract, or scent of berry, cherry, cinnamon, clove, ginger, grapefruit, lemon, lime, nutmeg, vanilla, lavender, or orange, or any combinations thereof. In certain embodiments, the odorant agent comprises a mint oil or mint extract. In one embodiment, the mint is peppermint. In one embodiment, the odorant agent is derived from Mentha piperita L. In some embodiments, the odorant agent comprises vanilla. In some embodiments, the odorant comprises a lemon oil. The scented insert may comprise up to 1%, up to 3%, up to 5%, up to 7%, up to 10%, up to 12%, up to 15%, or up to 20% of the odorant agent by weight. In certain embodiments, the scented insert comprises between 1% to 20%, between 5% to 15%, between 8% to 12%, or between 9% to 11% of the odorant agent by weight. In certain embodiments, the scented insert comprises about 10% of the odorant agent by weight.

In some embodiments, the polymer is a copolymer. The polymer may comprise vinyl acetate. The polymer may be a vinyl acetate copolymer. The polymer may comprise ethylene. In one embodiment, the polymer is ethylene vinyl acetate. The scented insert may comprise up to 99%, up to 97%, up to 95%, up to 92%, up to 90%, up to 88%, up to 85%, or up to 80% polymer by weight. In certain embodiments, the scented insert comprises from 99% to 80%, from 95% to 85%, from 92% to 88%, or from 91% to 89% polymer by weight. In certain embodiments, the scented insert comprises about 90% polymer by weight.

In one embodiment, the scented insert consists essentially of odorant agent and polymer.

The scented insert may be any suitable shape. The insert may generally have the shape of a polygon with three, four, five, six, seven, eight, nine, ten, eleven, twelve, thirteen, fourteen, fifteen, or more sides. The insert may be a triangle; quadrilateral, such as a square, rectangle, rhombus, parallelogram, trapezoid, rhomboid, kite; a pentagon; a hexagon; a heptagon; a octagon; a nonagon; a decagon; a ring; a prism, such as a cube; a sphere; a pyramid, such as a right pyramid, a rectangular pyramid, a rhombic pyramid, or a star pyramid; a cone; a torus; a cylinder; a letter; a symbol; or a word. The insert may be in the shape of a star, for example a three-pointed star, four-pointed star, five-pointed star, six-pointed star, seven-pointed star, eight-pointed star, nine-pointed star, ten-pointed star, eleven-pointed star, twelve-pointed star, thirteen-pointed star, fourteen-pointed star, fifteen-pointed star, or a star with greater than fifteen points. The star may be symmetrical, or may not have a plane of symmetry. The insert may be generally round, for example be circular or ovoid. In some embodiments, the insert is shaped to emulate another object, for example an animal, plant, or inanimate object. In some embodiments, the insert comprises one or more holes or cutouts.

The insert may have a generally consistent thickness, for example having a thickness that deviates less than 10%, less than 5%, less than 2%, less than 1%, or less than 0.5%. In other embodiments, the insert comprises at least one point that deviates greater than 15%, greater than 20%, greater than 25%, greater than 30%, or greater than 35% in thickness from at least one other point. In some variations, the insert is relatively flat. In other variations, the insert is generally not flat. The insert may comprise one or more letters or symbols, for example located on one or more surfaces of the insert. The one or more letters or symbols may be embossed, punched, or etched into the surface.

In some embodiments, the insert is translucent. In some embodiments, the insert is transparent.

In some embodiments, the plurality of solid dosage forms is packaged in any suitable container. In some embodiments, the container is, for example, a bottle or a jar. In some embodiments, the container is of any suitable material, such as plastic or glass. In some embodiments, the container comprises a lid, for example a cap, such as a snap-on cap or a screw top cap. In some embodiments, the lid is fully removable, for example, the lid is completely separated from the container. In some embodiments, the lid is connected to the container, for example by a piece of material that is attached to both the cap and the container, such that when the cap is removed from the opening of the container it is still connected to the container.

In some embodiments, the container comprises a flexible packaging material. In some embodiments, the flexible packaging material comprises post-consumer recycled material. In some embodiments, the container comprises the flexible packaging material and a closure. In some embodiments, the closure is rescalable. In some embodiments, the container comprises the flexible packaging material and a gusset flat bottom. In some embodiments, the container comprises the flexible packaging material, the closure (e.g., the resealable closure), and the gusset flat bottom, wherein the flexible packaging material comprises post-consumer recycled material.

In some embodiments, the container comprises one or more plastics. In some embodiments, the container is essentially free of one or more bisphenol compounds. In one embodiment, the container is essentially free of bisphenol A. In some embodiments, the container is any suitable color, or may comprise a plurality of colors, or is transparent or translucent. In some embodiments, at least a portion of the bottle is transparent or translucent. In one embodiment, the container is transparent. In some embodiments, the container comprises a bottle and a removable screw-top lid, wherein the bottle and lid comprise plastic and are essentially free of bisphenol A. In one embodiment, the bottle is transparent and the lid is white.

In some embodiments, the container comprises one or more markings, such as a label or design. In some embodiments, the markings comprise one or more words, symbols, instructions, slogans, or combinations thereof. In some embodiments, the one or more markings is any suitable color, or a plurality of colors. In one embodiment, the markings are white.

As described above, in some embodiments the container comprises a scented insert. For example, a scented insert is included inside the bottle, along with the composition.

In some embodiments, the container is further packaged in one or more additional containers. In some embodiments, the additional container is a box, such as a cardboard box, for example a corrugated cardboard box. For example, in some embodiments the composition is packaged in a container comprising a bottle and a removable screw-top lid, wherein the container is further packaged in an additional container comprising a cardboard box. In some embodiments, the additional container is any suitable color or shape, and may comprise one or more markings. In some embodiments, the additional container is a cardboard box comprising an attached lid that partially tucks into the base, wherein the box comprises a white exterior and a yellow interior, and one or more markings.

In some embodiments, the packaging further comprises instructions, for example one or more pamphlets, sheets, booklets, or other written media. In some embodiments, the instructions are made of any suitable material, such as paper.

Provided herein is a kit, comprising a plurality of solid dosage forms described herein, and a container. In some embodiments, the kit further comprises instructions. In some embodiments, the plurality of solid dosage forms is packaged in a container. In some embodiments, the container comprises a bottle and a removable screw-top lid, wherein the container comprises plastic. In some embodiments, the container further comprises a scented insert. The scented insert is translucent. In some embodiments, the container is further packaged within an additional container, wherein the additional container comprises a cardboard box. In certain embodiments, the kit further comprises instructions. In some embodiments, the instructions comprise daily administration. In some embodiments, the administration is oral.

### EXAMPLES

### Example 1: Effect of Tablet Geometry on Manufacture of a Multi-Tablet-In-Capsule Dosage Form

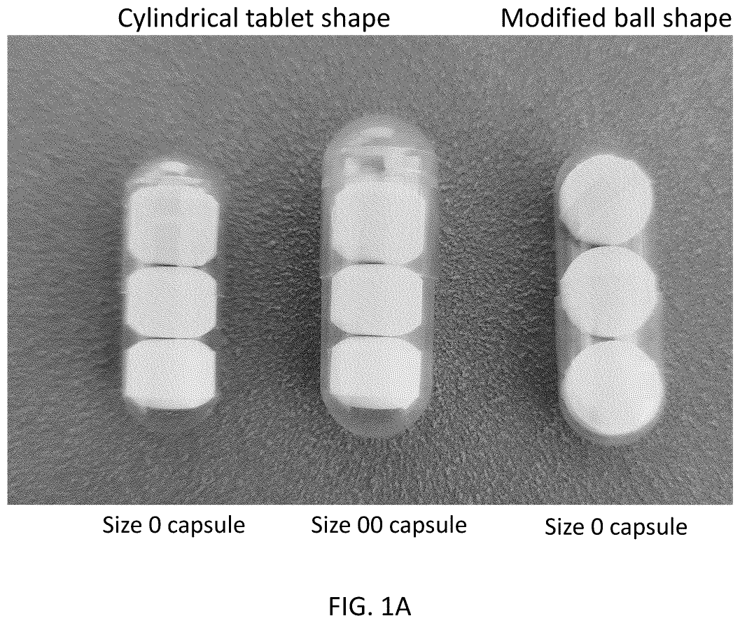

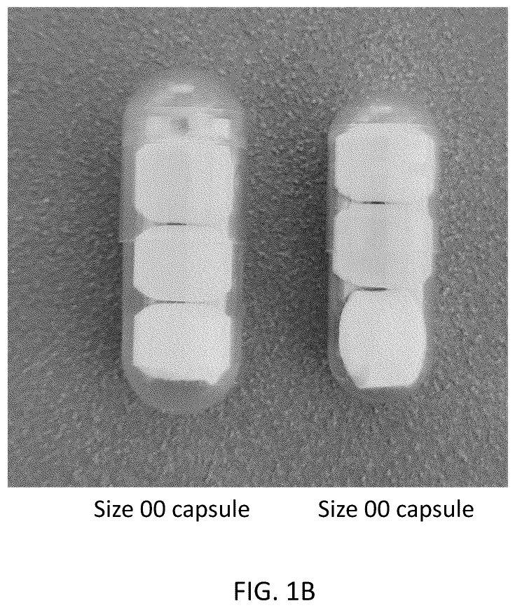

A tablet-in-capsule dosage form was prepared containing three tablets in a single capsule. The effect of tablet geometry on capsule filling was evaluated. Tablets were prepared having a cylindrical shape with a diameter of 7.04 mm or a modified ball shape with a diameter of 6.2 to 6.9 mm. The tablets were prepared by direct compression of powdered excipients using a tablet press. Different shapes of tablets were prepared by using different punches and dies in the tablet press. The tablet matrix contained MCC, ascorbyl palmitate powder, silica, Hypromellose, psyllium husk and oat fiber. The tablets were coated with a thin coating solution (AquaPolish® F clear 094.50 (HPMC, HPC, sunflower oil or Aquapolish F clear 299.13E sodium alginate, MCC, HPC, sunflower oil) using a tablet coating pan. The capsule was either a size 0 or a size 00 and made of hypromellose (Lonza Capsugel; VCaps® Plus). The size 0 capsule was selected as the volume was considered suitable for encapsulating three cylindrical tablets stacked with flat surfaces in-parallel or three spherical tablets and without substantial excess space. Although a larger capsule (size 00) also encapsulates the three tablets, it was observed to contain residual space once filled. See FIG. 1A.

Capsule filling was performed by a capsule filling machine that positioned multiple capsules vertically, removed the capsule cap, filled the capsule body with the tablet that are filled into the capsule from three different hoppers, appended the capsule cap to the capsule body, and expelled the filled capsule from the machine with in-line weight checks. When filling the capsule, it was observed that the cylindrical tablets would enter the capsule sometimes with the circular flat-end towards the bottom of the capsule and sometimes with the rounded side towards the bottom of the capsule. See FIG. 1B. In filling a capsule size 0, if one cylindrical tablet entered the capsule with rounded side towards the bottom, then the capsule would over-fill and be ejected without proper capping. If the larger size 00 capsule was used, the three tablets would fit even if one was stacked vertically. However, this was less desirable as it resulted in a mixture of capsules (i.e., a subset having tablets arranged with flat surfaces in-parallel and a subset having tablets arranged with at least one vertical relative to the others). In contrast, it was observed that the modified ball shape filled the size 0 capsule without risk of ejection regardless of which direction the tablet entered the capsule.

Thus, the modified ball shape was considered advantageous as it provided easier capsule filling (i.e., fewer ejected tablets) while maximally filling the volume of the capsule.

### Example 2: Formulation Parameters Impact Release Profile of Multi-Tablet-In-Capsule Dosage Form

A dosage form of a single spherical tablet in a single capsule was prepared and the effect of formulation parameters on release profile of the active ingredient in the tablets was evaluated. It was considered that including a rate controlling polymer in the tablet matrix or a thicker coating would extend or delay release of an encapsulated active ingredient. However, all ingredients selected were required to meet the criteria of plant-based (i.e., vegan) and qualifying as “clean” (e.g., recognizable by consumers, non-GMO ingredients, low in heavy metals, traceable to the final place of manufacture).

The tablet formulation contained a rate controlling polymer in the matrix and a coating that was a blend of rate controlling polymers (HPC and MCC), sodium alginate, and sunflower oil. The capsule formulation was made of hypromellose (VCap Plus, Lonza/Capsugel). The tablet compaction and capsule filling was performed as described in Example 1.

Several dosage forms were prepared having different formulation parameters as indicated in Table 3A. Formula A and B contained the same amount of melatonin. Formula B and C contained the same polymer levels. Formula C and D contained 1.25 mg of melatonin with different polymer levels. Formula E contained a coating.

TABLE 3A
formulation parameters for dosage forms containing melatonin

| Formula | A | B | C | D | E |
|---|---|---|---|---|---|
| Melatonin | 1 mg | 1 mg | 1.25 mg | 1.25 | 1 mg |
| Polymer | 10% | 15% | 15% | 17% | 17% |
| Coating | none | none | none | none | 1% |

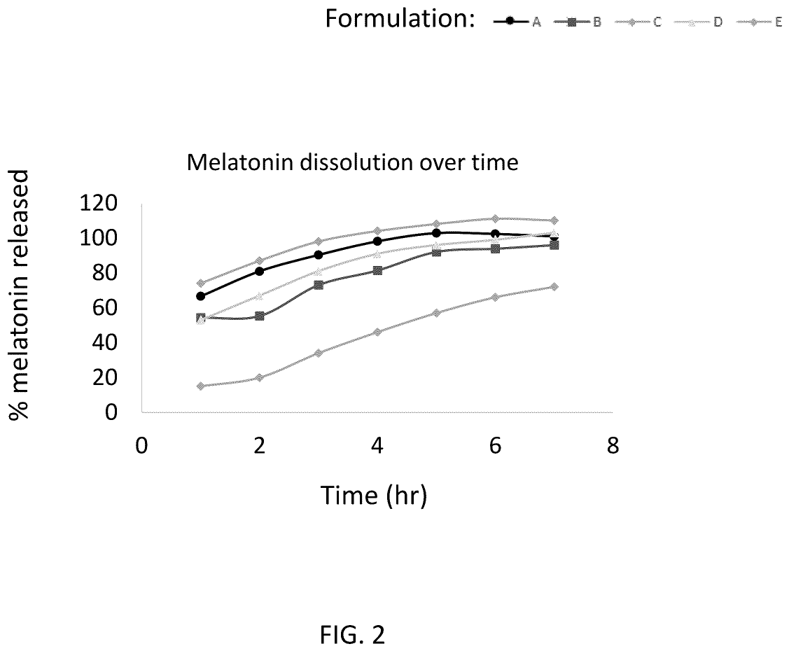

Dissolution of the tablets was evaluated using the following method. The tablets were placed in a stationary basket/paddle apparatus containing 1000 mL, 0.1N HCl at a speed of 100 rpm. After 1 hour, the dissolution media was changed to gastrointestinal phosphate buffer (pH 6.8). The amount of active in the dissolution media was measured by HPLC. The dissolution profiles as measured is provided in Table 3B as cumulative percent release over time and shown in FIG. 2.

TABLE 3B
dissolution profile for formulations of Table 3A shown as
cumulative percent release of dose of melatonin over time

| | | | | | |
|---|---|---|---|---|---|
| Time | A | B | C | D | E |

| | | | | | | |
|---|---|---|---|---|---|---|
| 30 | min | 66.6 | 54.3 | 74 | 53 | 15 |
| 1 | hr | 80.9 | 55.3 | 87 | 67 | 20 |
| 2 | hr | 90.3 | 73 | 98 | 81 | 34 |
| 3 | hr | 98.1 | 81.4 | 104 | 91 | 46 |
| 4 | hr | 102.8 | 92 | 108 | 96 | 57 |
| 5 | hr | 102.3 | 93.8 | 111 | 99 | 66 |
| 6 | hr | 100.9 | 95.9 | 110 | 103 | 72 |

Formulation B had a slower release of melatonin compared to formulation A and formulation D had slower release of melatonin compared to formulation C. Formulations A and B contained the same amount of melatonin, but formulation B contained a higher amount of rate controlling polymer. Similarly, formulations C and D contained the same amount of melatonin, but formulation D had an increased amount of rate controlling polymer. These results indicate a higher amount of polymer in the matrix slows release of an active ingredient. It was considered that the rate controlling polymer swells as it becomes hydrated over time and results in an increased diffusion time for encapsulated active that slows release at later time points.

Formulation B had slower release of melatonin compared to Formulation C. Formulations B and C contained the same amount of rate controlling polymer, but formulation C contained a higher amount of melatonin. This result indicates a higher amount of active ingredient in the matrix increases the rate of release. It was considered that a higher amount of active in the tablet results in more being present at the surface of the tablet, thereby resulting in faster release.

Formulation E had the slowest release of melatonin. This result indicates that including a coating slows release of active ingredient in the matrix, particularly at early time points.

Together, these data demonstrate that release properties of the tablets are tuned by altering the amount of rate controlling polymer in the tablet matrix, the amount of coating relative to matrix, and the amount of active ingredient present in the matrix.

### Example 3: Evaluation of Formulation Parameters on Release Profile for Tablets Containing Melatonin

The effect of rate controlling polymer and coating on dissolution of tablets containing 1 mg, 1.25 mg, or 3 mg melatonin was evaluated.

The tablets contained the ingredients shown in Table 4.

TABLE 4
Ingredients for melatonin tablets

| Ingredient | Function |
|---|---|
| Melatonin | Active |
| MCC | Diluent |
| CompactCel ® F 900.25 DIS | Disintegrant |
| (psyllium husk + oat fiber) | |
| Ascorbyl Palmitate Powder | Lubricant |
| Silica | Glidant |
| Spirulina extract | Color |
| Hypromellose (Methocel ™ | Rate controlling polymer |
| E15) | (RCP) |
| Hypromellose (Methocel ™ | RCP |
| K4M) | |
| HPMC, HPC, sunflower oil | Coating |
| with or without acetylated | |
| starch | |

The formulation for the tablets is shown in Table 5.

TABLE 5
Ingredients used in formulation of melatonin tablets (values are the mass percent of ingredient per tablet)

| | | | | | | 1.25 mg | | | | | | | | | |
|---|---|---|---|---|---|---|---|---|---|---|---|---|---|---|---|
| | | | | | | melatonin | | | | | | | | | |
| | 1 mg melatonin tablet | | | | | tablet | | 3 mg melatonin tablet | | | | | | | |
| | LB- | LB- | LB- | LB- | LB- | LB- | LB- | LB- | LB- | LB- | LB- | LB- | LB- | LB- | LB- |
| | 0362 | 041- | 117- | 060- | 1262 | 056- | 057- | 088- | 111- | 117- | 086- | 098- | 100- | 108- | 1272 |
| Ingredient | 2C | 22 | 22 | 22 | 2B | 22 | 22 | 22 | 22 | 22 | 22 | 22 | 22 | 22 | 2B |
| Melatonin | 1 mg | 1 mg | 1 mg | 1 mg | 1 mg | 1.25 | 1.25 | 3 | 3 | 3 | 3 | 3 | 3 | 3 | 3 |
| MCC | 20-30% MCC levels are adjusted | | | | | | | | | | | | | | |
| | to keep tablet weight same (before coating) | | | | | | | | | | | | | | |
| psyllium | — | — | — | — | — | — | — | — | — | — | — | — | — | — | — |
| husk + | | | | | | | | | | | | | | | |
| oat fiber | | | | | | | | | | | | | | | |
| Ascorbyl | 0.4% | 0.4% | 0.4% | 0.4% | 0.4% | 0.4% | 0.4% | 0.4% | 0.4% | 0.4% | 0.4% | 0.4% | 0.4% | 0.4% | 0.4% |
| Palmitate | | | | | | | | | | | | | | | |
| Powder | | | | | | | | | | | | | | | |
| Silica | 0.1% | 0.1% | 0.1% | 0.1% | 0.1% | 0.1% | 0.1% | 0.1% | 0.1% | 0.1% | 0.1% | 0.1% | 0.1% | 0.1% | 0.1% |
| Methocel ™ | — | — | 5% | — | 10% | | | 10% | 10% | 5% | 10% | 13% | 10% | 10% | 8% |
| E15 | | | | | | | | | | | | | | | |
| Methocel ™ | 10% | 15% | 15% | 17% | 8% | 15% | 17% | 10% | 12% | 15% | 10% | 12% | 8% | 8% | 8% |
| K4M | | | | | | | | | | | | | | | |
| Coating | — | — | — | 1% | 0.62% | — | — | — | — | — | — | — | — | 1% | 1% |
| Coating + | — | — | — | — | — | — | — | — | — | — | 1% | 1% | 1% | — | — |
| acetylated | | | | | | | | | | | | | | | |
| starch | | | | | | | | | | | | | | | |

Dissolution of the tablets was evaluated under gastrointestinal dissolution conditions using the following method. The tablets were placed in an apparatus with basket/paddle containing 1000 mL, 0.1N HCl at a speed of 100 rpm and temperature of 37° C. After 1 hour, the dissolution media was changed to gastrointestinal phosphate buffer (pH 6.8). The amount of melatonin in the dissolution media was measured by HPLC.

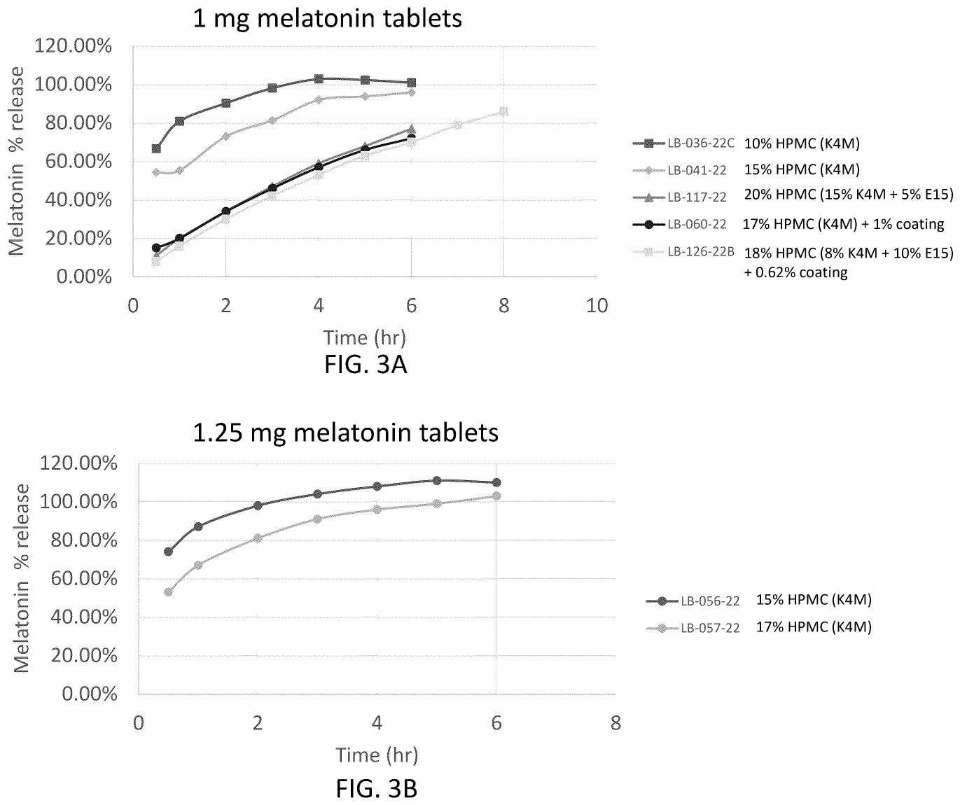

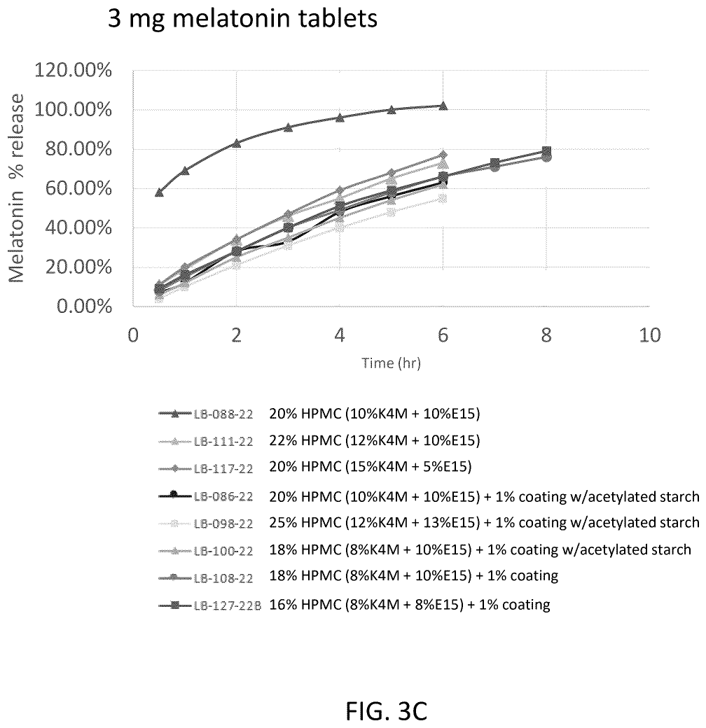

The dissolution profile for the tablets containing 1 mg, 1.25 mg, and 3 mg melatonin is shown in FIGS. 3A-3C respectively. Together, these data show the rate of dissolution was reduced for tablets having an increased amount of rate controlling polymer (Methocel™ E15 and/or Methocel™ K4M) in the matrix, while the initial burst release was reduced for tablets having a coating.

### Example 4: Formulation of a 3-Tablet-In-1-Capsule Dosage Form Containing Melatonin for Improving Sleep

A 3-tablet-in-1-capsule dosage form was developed to improve sleep in subjects following oral administration of a single capsule about 1 hour prior to bedtime. Each tablet was formulated to contain melatonin in an amount sufficient to provide a total of about 5 mg per capsule. The dose was selected as it is standard among commercially available melatonin products and has been shown to have a clinical effect (see Nagtegaal J E, et al J Sleep Res. 1998; 7 (2): 135-143; Sadeghniiat-Haghighi, K. et al. Journal of circadian rhythms vol. 6 10. 29 Oct. 2008; Kayumov L, et al Psychosom Med. 2001 63 (1): 40-8). The tablets each contained 20% overage of melatonin.

The tablets were designed to have a different release profile. Tablet 1 was designed for rapid release of melatonin (e.g., within about 1 hour following oral administration) by including a disintegrant and no rate controlling polymer in the matrix. Tablets 2 and 3 were designed for extended release of a moderate dose of melatonin (i.e., 3 mg melatonin) over a duration of about 6-8 hours and a lower dose of melatonin (i.e., 1 mg melatonin) over a duration of about 3 hours following oral administration respectively by including a mixture of grade E15 and grade K4M Hypromellose in the matrix. These grades differ in molecular weight. Tablet 2 was expected to have a higher initial burst release as compared to Tablet 3 due to the presence of a higher dose of active in the tablet. The tablets contained a different amount of a colorant or no colorant in order to have different colors. The tablets were prepared to have different colors in order to readily distinguish the tablets, which in turn improved quality control and efficiency during the manufacturing process.

The dosage form was designed to yield an instant release of melatonin to shorten sleep onset latency and an extended release of melatonin to mirror the circadian rhythm of endogenous melatonin production and release. In most healthy adults, a daily sleep pattern is regulated by endogenous melatonin production that begins increasing shortly after dark, reaches a peak in the middle of the night (e.g., between 2-4 AM), and then declines throughout the rest of the night to reach a low point shortly before an individual awakens (see, e.g., Grivas T B and Savvidou O D. Scoliosis. 2007 2:6). Without being bound by theory, the inclusion of an instant release component provides an amount of melatonin following ingestion that reduces the time required for an individual to fall asleep. However, as exogenously administered melatonin has poor bioavailability and a short half-life due to rapid processing and removal from the body (see Harpsoe N, et al. Eur J Clin Pharmacol. 2015 71 (8): 901-909), it was considered that the instant release component alone would not support restful sleep throughout the duration of a night. Thus, it was considered beneficial to include an extended release component to ensure that melatonin continues to be released over the course of several hours, thereby mirroring endogenous melatonin fluctuations. The extended release component of the dosage form was designed to mirror the endogenous melatonin release of a healthy individual by peaking around two hours after ingestion and then gradually tapering off within 6 hours after ingestion, to be metabolized by the body prior to the individual awakening for the day.

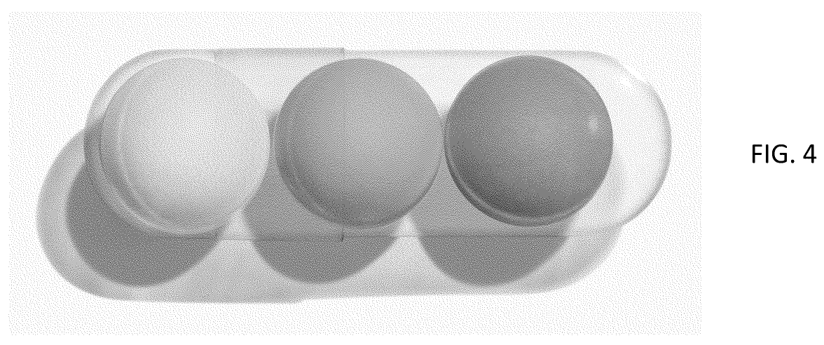

A visual of the dosage form is shown in FIG. 4. The dosage form contained a clear size 0 capsule with 3 spherical (modified ball shape) tablets. The specification for the three tablets is provided in Table 6. The capsule was made of hypromellose (VCap Plus, Lonza/Capsugel), which constituted 96 mg and 15.53% by weight of the capsule.

TABLE 6
Dosage form for improving sleep

| | | Tablet 1 -1 | | Tablet 2 - 3 | | | |
|---|---|---|---|---|---|---|---|
| | | mg immediate | | mg extended | | Tablet 3 - 1 | |
| | | release | | release | | mg extended | |
| | | (darkest blue, | | (medium blue, | | release (white, | |
| | | modified ball | | modified ball | | modified ball | |
| | | 6.40 mm) | | 6.40 mm) | | 6.40 mm) | |
| Raw Material Name | Function | Input | % | Input | % | Input | % |
| Melatonin | Active | 1.20 | 0.19 | 3.60 | 0.58 | 1.20 | 0.19 |
| MCC | Diluent | 159.10 | 25.74 | 138.72 | 22.45 | 141.40 | 22.88 |
| CompactCel ® F 900.25 DIS | Disintegrant | 6.00 | 0.97 | — | — | — | — |
| (psyllium husk + oat fiber) | | | | | | | |
| Ascorbyl Palmitate Powder | Lubricant | 2.60 | 0.42 | 2.60 | 0.42 | 2.50 | 0.40 |
| Silica | Glidant | 0.60 | 0.10 | 0.60 | 0.10 | 0.60 | 0.10 |
| Spirulina extract | Color | 0.50 | 0.08 | 0.18 | 0.03 | — | — |
| Hypromellose (grade E15) | RCP | — | — | 9.15 | 1.48 | 9.15 | 1.48 |
| Hypromellose (grade K4M) | RCP | — | — | 9.15 | 1.48 | 9.15 | 1.48 |
| AquaPolish ® F clear 094.50 | Coating | 4.00 | 0.65 | 10.00 | 1.62 | 10.00 | 1.62 |
| (HPMC, HPC, sunflower oil) | | | | | | | |
| Total per tablet | | 174.00 | 28.16 | 174.00 | 28.16 | 174.00 | 28.16 |

input = mg/capsule
% = percentage by weight per capsule

The dissolution profile for each tablet of the dosage form was evaluated as described in Example 3.

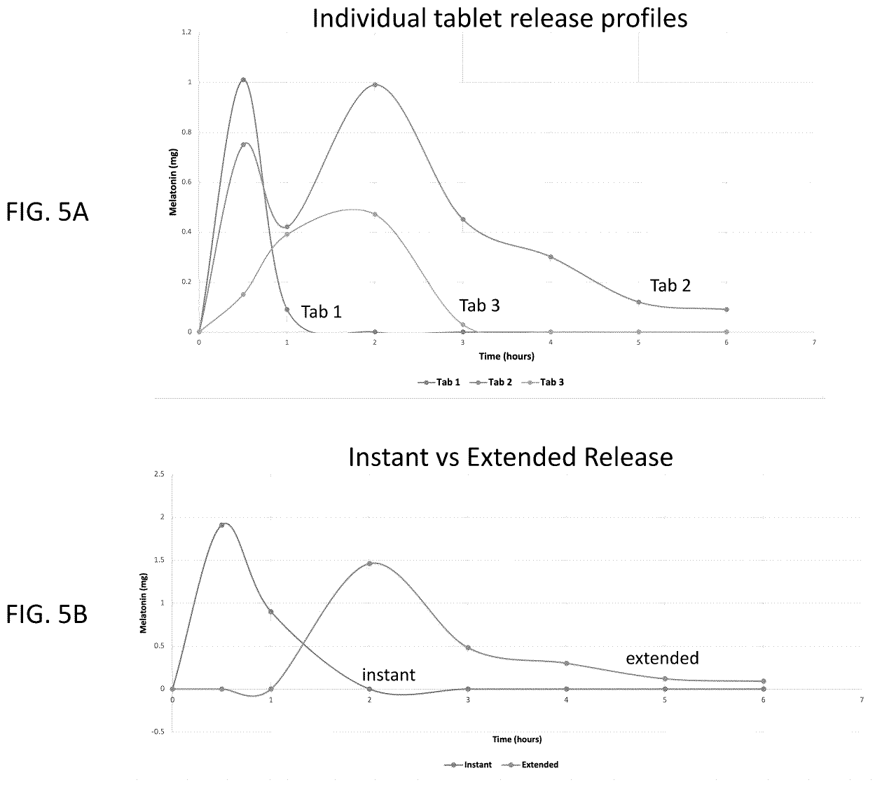

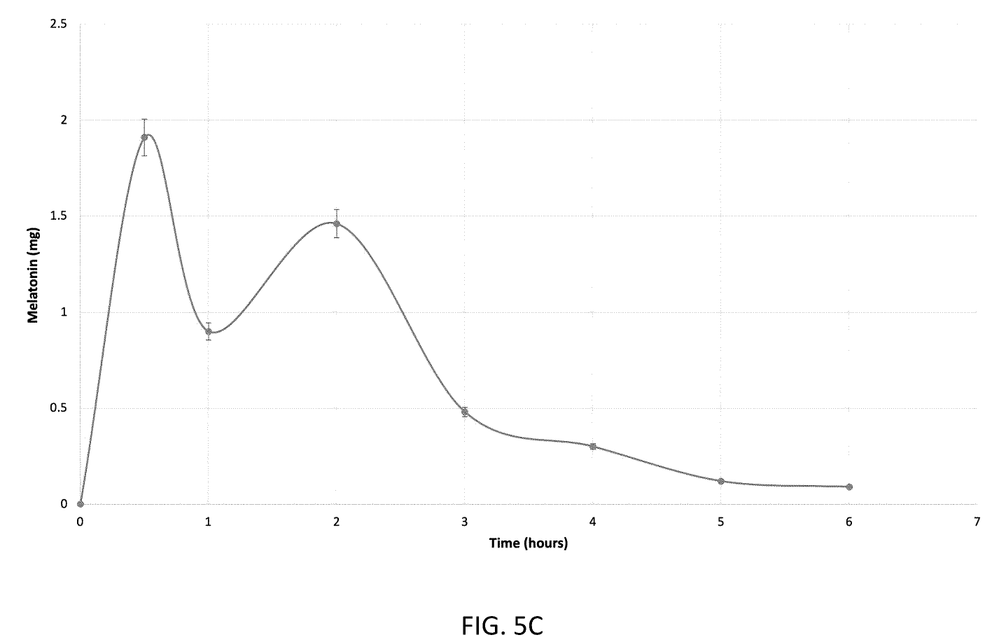

As shown in FIG. 5A, tablets 1, 2, and 3 resulted in complete release of encapsulated melatonin by about 1 hour (tablet 1), 3 hours (tablet 3), and 6-8 hours (tablet 2). The release profile for tablet 2 showed an initial burst release followed by a longer duration release of encapsulated melatonin over the duration of about 6-8 hours. The release profile for tablet 3 showed an extended release over the duration of about 3 hours. FIG. 5B provides the dissolution profile for tablet 1 as compared to the dissolution profile of tablets 2 and 3 combined. FIG. 5C provides the dissolution profile for the capsule.

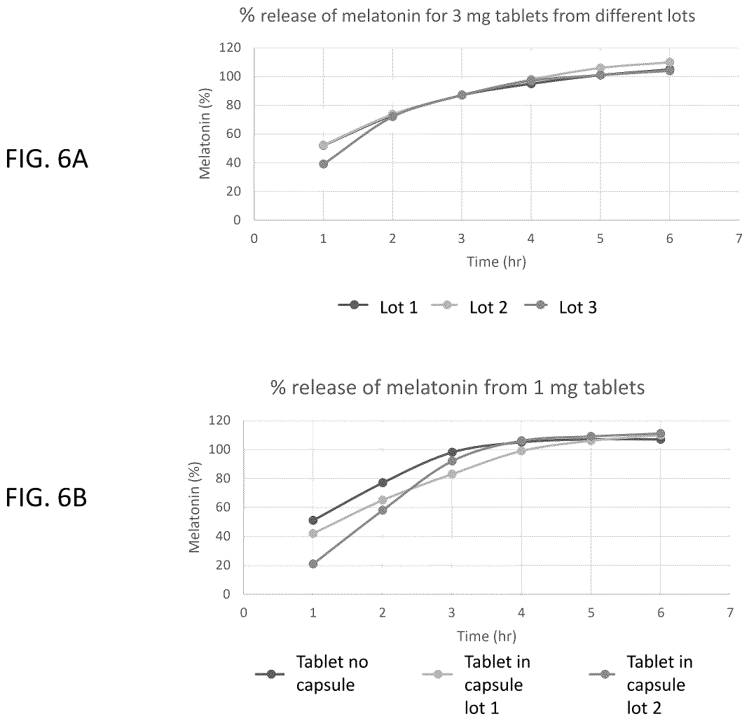

Dissolution of 3 mg tablets produced in three separate lots was evaluated. As shown in FIG. 6A, the dissolution profile was substantially the same (i.e., with less than 10% variability) for tablets from the separate lots, indicating consistent release properties between different lots of the dosage form.

Additionally, the dissolution profile of a 1 mg tablet compared to a 1 mg tablet in a capsule (2 separate lots) was evaluated. As shown in FIG. 6B, the dissolution profile was similar for the tablets whether in a capsule or not.

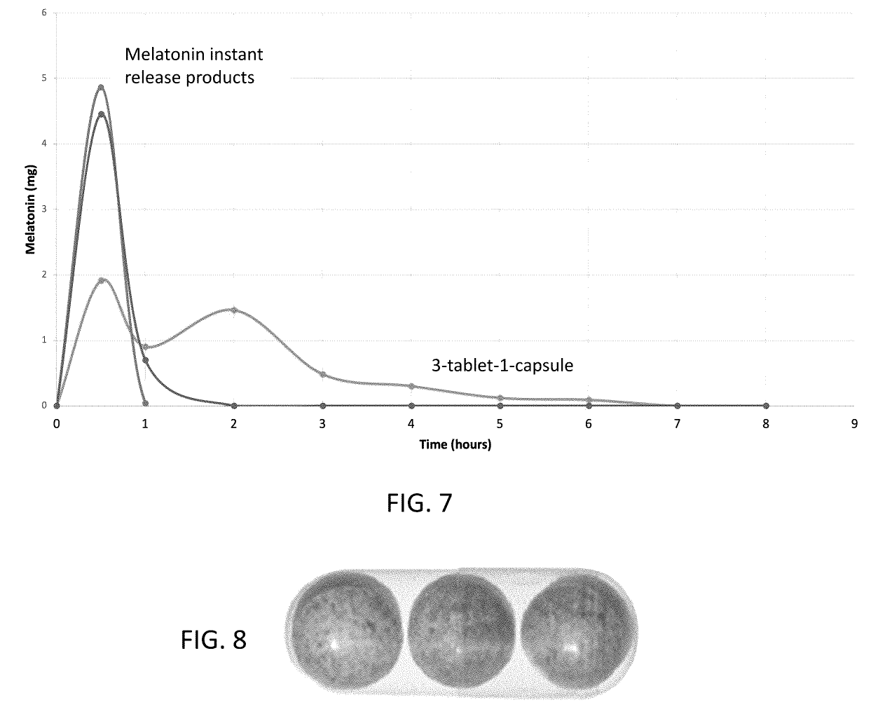

The dissolution profile for the dosage form was compared to two commercial melatonin products (a fast melt tablet and a standard tablet, each containing 5 mg melatonin). As shown in FIG. 7, the commercial melatonin products resulted in complete release of encapsulated melatonin within about 1-2 hours. In contrast, the dosage form resulted in an extended release profile over about 6 hours.

Together, these results show the excipients of each tablet in the 3-tablet-1-capsule dosage form (e.g., the amount of rate controlling polymer and active in the matrix) may be modified to provide a customized dissolution profile, such as for release of melatonin over a typical duration of nighttime sleep.

### Example 5: Formulation of a 3-Tablet-In-1-Capsule Dosage Form Containing Nutraceuticals for Stress Reduction

A 3-tablet-in-1-capsule dosage form was developed for administration of three nutraceuticals identified for reducing stress. The three nutraceuticals selected were saffron (Crocus sativus L.) stigma extract (affron®), ashwagandha (Withania somnifera) root/leaf extract (Shoden®), and L-theanine (Suntheanine®). The amount of the nutraceutical per capsule was 28 mg saffron, 80 mg ashwagandha, and 200 mg L-theanine. The dosage was selected to provide a subject with an effective dose if ingesting the capsule dosage form once per day (see Hidese, et al (Nutrients. 2019 Oct. 3; 11 (10): 2362); Lopresti, et al (Medicine (Baltimore). 2019 Scp; 98 (37): e17186)); Kell, et al (Complement Ther Med. 2017 August; 33:58-64). Additional ingredients in the dosage form included hypromellose, cellulose, ascorbyl palmitate, hydroxypropyl cellulose, silica, psyllium husk, a vegetable-based tablet coating, and oat fiber.

Formulation of the tablets encapsulated in the capsule was selected such that each tablet had a different dissolution profile for the nutraceuticals following oral administration. Tablet 1 was designed for rapid dissolution of the nutraceuticals, i.e., in about 1-2 hours, by including a disintegrant and no rate controlling polymer in the matrix (“immediate release”). Tablet 2 was designed for dissolution of the nutraceuticals over an intermediate duration, i.e., within about 4 hours (“intermediate release”). Tablet 3 was designed for dissolution of the nutraceuticals over the longest duration, i.e., about 8 hours (“extended release”). Tablet 2 and 3 contained E15 and K4M Hypromellose in the tablet matrix to slow dissolution of the encapsulated active ingredients, but the amount of Hypromellose was higher in the matrix of tablet 3 to produce the slowest dissolution profile of the three tablets. Additionally, the dose of saffron varied for the three tablets. This was done so the color of the tablets was distinct. A visual of the dosage form is shown in FIG. 8. The specification for the three tablets is provided in Table 7.

Tablet 1 was designed to deliver a dose per capsule of 66.67 mg L-theanine (115% overage), 4.0 mg saffron (120% overage), and 26.67 mg ashwagandha (110% overage); Tablet 2 was designed to deliver a dose per capsule of 66.67 mg L-theanine (115% overage), 9.0 mg saffron (120% overage), and 26.67 mg ashwagandha (110% overage); and Tablet 3 was designed to deliver a dose per capsule of 66.67 mg L-theanine (115% overage), 15.0 mg saffron (120% overage), and 26.67 mg ashwagandha (110% overage). The capsule was designed to be taken once a day (in the morning) to provide all day stress relief.

Cortisol levels are regulated in a circadian rhythm. In most individuals, the levels peak shortly after waking, decline throughout the rest of the day, and reach a low point in the middle of the night (see Jones C and Gwennin C. Physiol Rep. 2021 8 (24): e14644; Russel G and Lightman S. Nat Rev Endocrinol. 2019 15 (9): 525-534). The dosage form was designed for consumption in the morning and to have a release profile of the active ingredients that normalizes endogenous cortisol levels in a manner that mimics the endogenous circadian rhythm. In particular, a release profile was selected such that a large portion of the active ingredients are released in the first few hours after ingestion and the remaining contents continue to be released for up to eight hours. The “frontloaded” release of the active ingredients was designed to normalize cortisol levels during a period of the day in which they are the highest in most individuals. Without being bound by theory, a release of the majority of the dose active ingredients in the first few hours after morning ingestion of the dosage form functions to normalize cortisol levels in a manner that mimics endogenous circadian rhythm.

The dosage form contained a clear size 00 capsule with 3 spherical (modified ball shape) tablets. The capsule was made of hypromellose (VCap Plus, Lonza/Capsugel), which constituted 122 mg and 15.48% by weight of the capsule.

TABLE 7
Dosage form for reducing stress

| | | Tablet 1 - | | Tablet 2 - | | Tablet 3 - | |
|---|---|---|---|---|---|---|---|
| | | immediate | | intermediate | | extended | |
| | | release | | release | | release | |
| | | (modified ball | | (modified | | (modified | |
| | | 6.9 mm) | | ball 6.9 mm) | | ball 6.9 mm) | |
| Raw Material Name | Function | Input | % | Input | % | Input | % |
| Suntheanine L-theanine (98%) | Active | 78.24 | 9.9. | 78.24 | 9.93 | 78.24 | 9.93 |
| Affron saffron | Active | 4.80 | 0.61 | 10.80 | 1.37 | 18.00 | 2.28 |
| Shoden ashwagandha | Active | 29.34 | 3.72 | 29.34 | 3.72 | 29.34 | 3.72 |
| MCC | Diluent | 66.42 | 8.43 | 59.92 | 7.60 | 33.32 | 4.23 |
| MCC CEOLUS | Diluent | 2.00 | 0.25 | 6.60 | 0.84 | 6.60 | 0.84 |
| CompactCel ® F 900.25 DIS | Disintegrant | 18.00 | 2.28 | — | — | — | — |
| (psyllium husk + oat fiber) | | | | | | | |
| Ascorbyl Palmitate Powder | Lubricant | 8.00 | 1.02 | 5.00 | 0.63 | 5.00 | 0.63 |
| Silica | Glidant | 5.70 | 0.72 | 4.20 | 0.53 | 4.20 | 0.53 |
| Hydroxypropyl cellulose (HPC | | 2.00 | 0.25 | 6.60 | 0.84 | 6.60 | 0.84 |
| SSL-SPF) | | | | | | | |
| Hypromellose (Methocel ™ | RCP | — | — | 9.40 | 1.19 | 13.50 | 1.71 |
| E15) | | | | | | | |
| Hypromellose (Methocel ™ | RCP | — | — | 9.40 | 1.19 | 26.70 | 3.39 |
| K4M) | | | | | | | |
| AquaPolish ® F clear 094.50 | Coating | 3.00 | 0.38 | 3.00 | 0.38 | 3.00 | 0.38 |
| (HPMC, HPC, sunflower oil) | | | | | | | |
| Sunflower oil | | 0.50 | 0.06 | 0.50 | 0.06 | 0.50 | 0.06 |
| Total per tablet | | 218 | | 223 | | 225 | |

input = mg/capsule
% = percentage by weight per capsule

The dissolution profile for each tablet of the dosage form was evaluated under gastrointestinal dissolution conditions. Dissolution was measured using the method described in Example 3. The amount of L-theanine release over time was quantified. L-theanine in the release media was measured by HPLC.

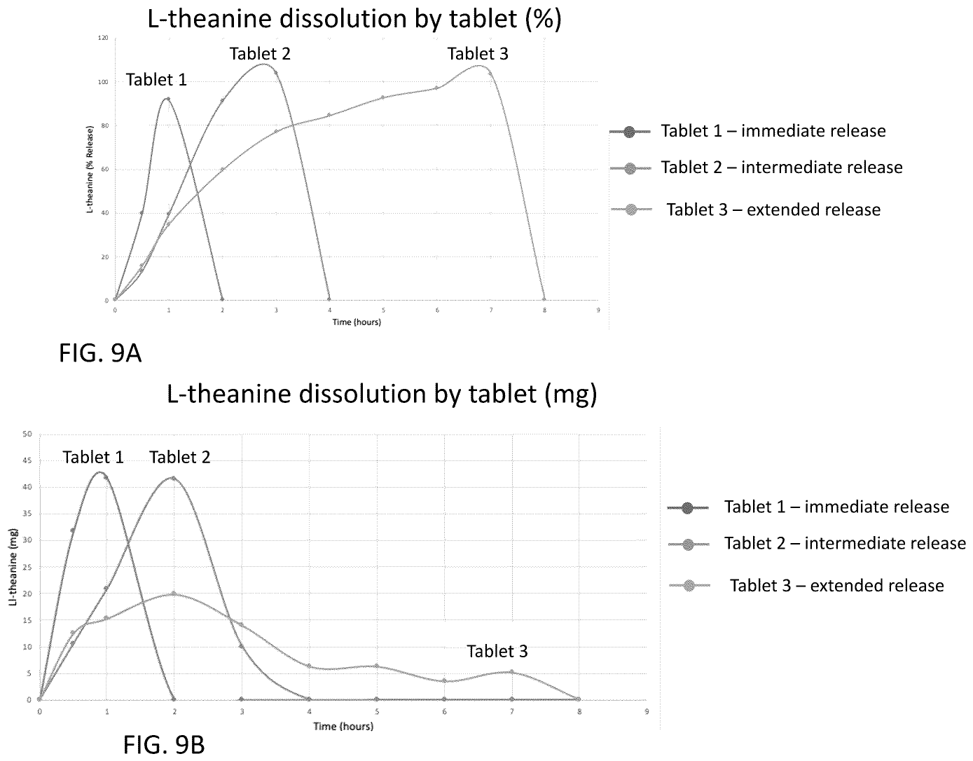

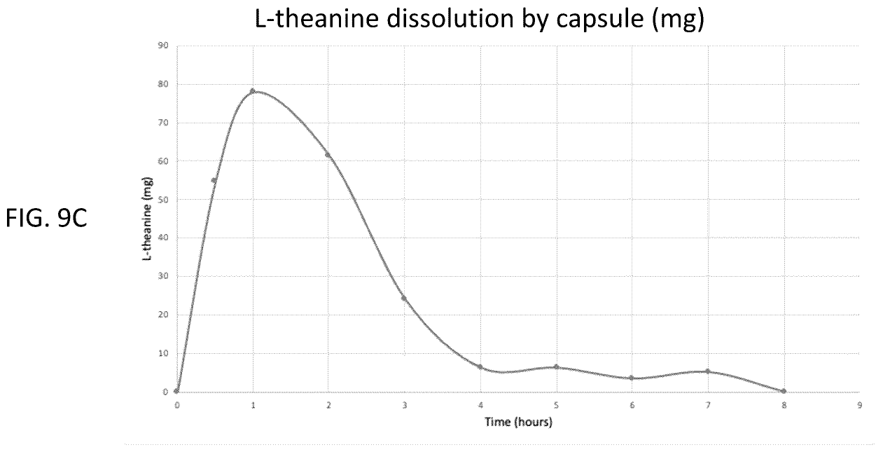

As shown in FIGS. 9A-9B, tablet 1 reached complete dissolution in about 2 hours, tablet 2 in about 4 hours, and tablet 3 in about 8 hours. The total amount of L-theanine released over time for the capsule under the dissolution conditions is shown in FIG. 9C.

Together, these results show the 3-tablet-1-capsule dosage form is amenable to encapsulation of multiple active ingredients and the excipients of each tablet (e.g., the amount of rate controlling polymer in the matrix) may be modified to provide a customized dissolution profile

### Example 6: Packaged Tablet-In-Capsule Dosage Form Remains Shelf-Stable for an Extended Duration

The stability of the multi-tablet-in-capsule dosage forms described in Example 4 (containing melatonin) and Example 5 (containing saffron, ashwagandha, and L-theanine) was evaluated over an extended duration.

The stability of the melatonin dosage form was evaluated as follows. The 3-tablets-in-1-capsule dosage form was stored in a clear 60 cc bottle with an induction seal and cap and an insert placed inside the bottle (30 count per bottle). The bottle contained an insert infused with natural vanilla flavors and medium chain triglycerides and imprinted with the words “This stays in the bottle, k?” The bottles were maintained at 25° C.±2° C. and room humidity 60%±5%. A sample was obtained at 3 months, 6 months, and 9 months and the capsules were evaluated for appearance (i.e., one tablet a blue color, one tablet a light blue color, and one tablet a white color, each ball shaped and filled in a clear capsule), weight per capsule, amount of melatonin per capsule, dissolution of melatonin per capsule, and microbial contamination. Dissolution was measured as described in Example 3.

Characterization data is provided in Table 8. Additionally, the capsules were observed to maintain appearance at each time point tested (3, 6, and 9 months). No microbial contamination was observed.

TABLE 8
Stability results for melatonin 3-tablet-1-capsule dosage form

| | Expected | | | | |
|---|---|---|---|---|---|
| Test | Range | Initial | 3 month | 6 month | 9 month |
| Weight | 556.2 mg-679.8 | 617.1-628.4 | 609.8-633.7 | 620.6-631.1 | 621.0-633.1 |
| variation | mg | | | | |
| Melatonin | 5 mg/tablet | 5.8 | 5.9 | 5.9 | 5.8 |
| (mg/capsule | (100-140%) | | | | |
| Dissolution | After 60 min | 54% | 58.4% | 57.6% | 53.8% |
| per capsule | After 120 min | 91% | 86.6% | 80.4% | 80.4% |
| (melatonin | After 180 min | 103% | 96.4% | 91.2% | 91.2% |
| release/5 mg | After 240 min | 110% | 101.8% | 99.0% | 99.0% |
| per capsule)* | After 300 min | 115% | 106.2% | 103.4% | 103.4% |
| | After 360 min - | 113% | 109.6% | 106.4% | 106.4% |
| | NLT 90% | | | | |
| Water activity | | 0.4262@25.02° C. | 0.4843@25.06° C. | 0.4942@25.02° C. | 0.5227@25.14° C. |

The stability of tablet 2 or tablet 3 described in Example 5 was evaluated as follows. A single tablet was encapsulated per capsule. The capsules were stored in a clear 100 cc bottle with a liner and cap (30 count per bottle). The bottle contained a citrus-scented insert imprinted with the words “This stays in the bottle, k?”. The bottles were maintained at 40° C.±2° C. and room humidity 75%±5%. The capsules were evaluated at 1 month for appearance (tablet 2: beige to yellow color ball shaped coated tablet with speckles filled in clear capsule; tablet 3: yellowish orange color ball shaped coated tablet with speckles filled in clear capsule), amount of L-theanine per capsule, and dissolution of L-theanine per capsule. Dissolution was measured as described in Example 5. Characterization data is provided in Table 9 (tablet 2) and Table 10 (tablet 3). The capsules were observed to maintain appearance.

TABLE 9
Stability results for stress 1-tablet-1-capsule
dosage form (intermediate release tablet)

| Test | Expected Range | Initial | 1 month |
|---|---|---|---|
| L-theanine | 66.7-100 mg | 81.51 | 78.27 |
| (mg/capsule) | | | |
| Dissolution per | % After 30 min | 25.5 | 28.5 |
| capsule* | % After 60 min | 56.7 | 53.7 |
| | % After 120 min | 91.0 | 81.7 |
| | % After 180 min | 110.8 | 103.4 |
| | % After 240 min | 119.1 | 113.2 |

TABLE 10
Stability results for stress 1-tablet-1-capsule
dosage form (extended release tablet)

| Test | Expected Range | Initial | 1 month |
|---|---|---|---|
| L-theanine | 66.7-100 mg | 79.82 | 75.34 |
| (mg/capsule) | | | |
| Dissolution per | % After 30 min | NA | 19.5 |
| capsule* | % After 60 min | 37.5 | 41.8 |
| | % After 120 min | 67.2 | 68.3 |
| | % After 180 min | 86.0 | 84.2 |
| | % After 240 min | 99.0 | 97.0 |
| | % After 300 min | 107.3 | 105.3 |
| | % After 360 min | 115.5 | 109.1 |
| | % After 420 min | 114.9 | 107.1 |
| | % After 480 min | 114.3 | 109.4 |

Together, these data show stability of the tablet-in-capsule dosage form is maintained following storage for an extended duration.

### Example 7: Study Design for Assessing Sleep Benefits of 3-Tablet-In-1-Capsule Supplement Containing Melatonin in Humans

The supplement described in Example 4 (referred to in this Example as the “sleep supplement”) is evaluated in healthy human subjects in a randomized, double blinded, and placebo-controlled crossover clinical trials to investigate the pharmacokinetics of the sleep supplement and its effects on sleep quality parameters.

The clinical study is performed with healthy young (age 18-40) male and female participants with poor sleep, as identified by a Pittsburgh Sleep Quality Index (PSQI) score >5. A PSQI score >5 is reported to have a diagnostic sensitivity of 89.6% and specificity of 86.5% (kappa=0.75, p less than 0.001) in distinguishing good and poor sleepers (see Buysse 1989; 28 (2): 193-213). Participants routinely taking melatonin supplements or other drugs known to affect sleep are excluded. Additional exclusion criteria include sleep disorders (e.g. delayed sleep phase disorder, sleep apnea, narcolepsy, or restless leg syndrome), major psychiatric illness, major physical illness, known or suspected hypersensitivity to melatonin, presence of major sleep disruptors at home (e.g. infants), night shift/rotational-shift work, receiving non-pharmacological treatment for sleep disorders (e.g. cognitive behavioral therapy), substance use disorder, recent (<1 month) increase in daily caffeinated drink consumption, consuming >14 standard alcoholic drinks per week, and those who are pregnant or lactating.

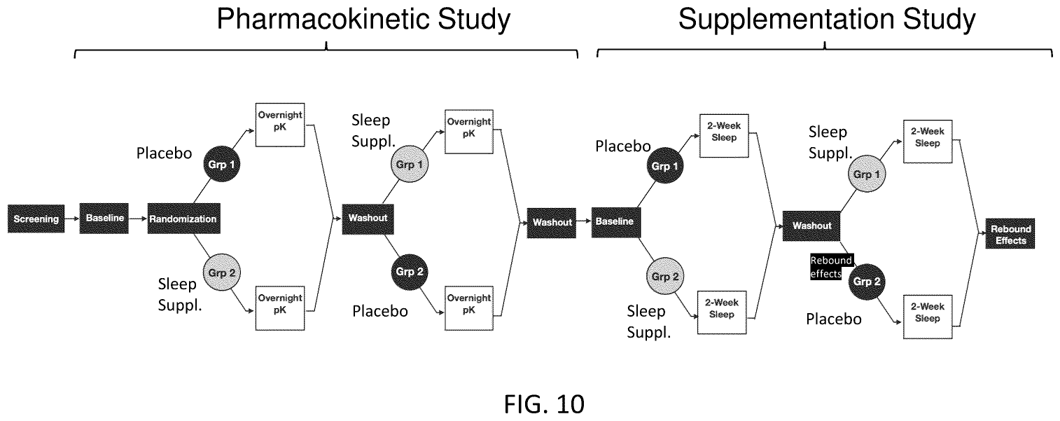

The clinical study is composed of two arms and uses a randomized, double-blind, placebo-controlled, crossover design, as diagrammed in FIG. 10. The participants are randomly assigned to a group that receives placebo or the sleep supplement. Following the period of receiving the placebo or sleep supplement, participants in each group undergo a wash-out period of 7 days. Participants thene complete another treatment in their assigned arm (FIG. 10). For example, if during the first treatment they consumed the sleep supplement, during the second treatment they consume the placebo. Because the study uses a crossover design, all participants experience both a placebo treatment period and a melatonin treatment period. Participants thereby act as their own controls. The study is to investigate the pharmacokinetics of the sleep supplement compared to normal fluctuations in endogenous melatonin (placebo) in human participants over a 10-hour nighttime period. The first study is referred to as the “pharmacokinetic study” in this Example. The purpose of this evaluation is to determine if consuming the sleep supplement increases melatonin above endogenous concentrations at night. The study is to measure sleep quality and other sleep variables over a two-week supplementation period using a wearable continuous monitoring device (Fitbit) and subjective surveys. The second study is referred to as the “supplementation study” in this Example. The pharmacokinetic study occurs before the supplementation study, as shown in FIG. 10.

The primary outcome for the pharmacokinetic study is melatonin AUC (area under the curve for serum concentration of melatonin over time) over a 10 hour night-time period in subjects receiving the sleep supplement. Comparison is made to placebo. The primary outcomes for the supplementation study are average sleep onset latency and efficiency measured over the two-week period. The secondary outcomes include sleep quality, quality of life, next-morning well-being, safety, and the presence of a “rebound effect,” as measured by subjective surveys. Comparison is made to placebo.

Study participants are randomized into a group that receives the sleep supplement (“test group”) and a group that receives placebo (“placebo group”). The placebo is visually identical to the sleep supplement and both the participants and the investigators are blinded as to which product is which. During the pharmacokinetic study, the pharmacokinetics of the sleep supplement is compared to the normal fluctuations in endogenous melatonin (placebo) over a 10 hour nighttime period. Participants report to the lab and consume either placebo or melatonin one hour before bedtime. Melatonin production is naturally stimulated by the onset of darkness and inhibited by the presence of light, whether natural or artificial (Grivas T B and Savvidou O D. Scoliosis. 2007 2:6). Therefore, the protocol is designed so that participants spend the night sleep in a darkened lab room and blood samples are taken continuously. By conducting this study in a darkened lab overnight, the effects of the sleep supplement on melatonin levels are compared to endogenous melatonin levels in the placebo group.

During the pharmacokinetic study, the test group and the placebo group respectively ingest a 200 ml drink with the sleep supplement or a visually identical placebo containing cellulose, hypromellose, vegetable-based tablet coating, ascorbyl palmitate, oat fiber, psyllium seed husk, silica, and spirulina extract (for color). Blood samples are taken over the course of 10 hours. Participants spend the night in a darkened lab. Participants are allowed to set their alarms and wake up in time to attend class or begin working. For collection of blood samples, 4 ml of whole blood are collected into a serum separation tube and centrifuged for 10 min (4000×g, 4° C.). Two one ml serum aliquots are frozen immediately at −20° C., before being transferred to a −70° C. freezer. Serum melatonin concentration is batch quantified at the end of the study via high-performance liquid chromatography (HPLC) equipped with a UV/VIS detector.

Next, at the beginning of the supplementation study, participants are provided a wearable device (Fitbit) and instructed on proper usage, as well as how to complete their daily sleep diary. Over the course of a one week baseline period, participants are continuously wearing the wearable device, which they regularly sync with their cellular phones. Participants are keeping a daily sleep diary (e.g. Consensus Sleep Diary Protocol, see Carney et al Sleep. 2012; 35 (2): 287-302). Next-morning well-being, sleep quality, and quality of life are measured by surveys.

During the supplementation study, the test group and the placebo group respectively consume one sleep supplement or the visually identical placebo approximately one hour before bedtime and attempt to sleep for at least six hours each night. Participants continue consuming one sleep supplement or placebo per day for two weeks, during which they continuously wear the wearable device (e.g. Fitbit), which they regularly sync with their cellular phones. During the supplementation phase, participants keep a daily sleep diary (e.g., Consensus Sleep Diary Protocol, see Carney et al Sleep. 2012; 35 (2): 287-302). Next-morning well-being, sleep quality, and quality of life are measured by surveys.

After the two week supplementation period, participants begin a one week washout period (FIG. 10). During washout period, first morning after the discontinuation, Fitbit data and survey data on well-being are collected to assess rebound effects. After washout, participants complete another two week supplementation period during which they consume whichever treatment they did not receive during the initial supplementation period (FIG. 10). For example, if during the first supplementation period they consumed the sleep supplement, during the second supplementation period they consume the placebo. Participants continue consuming their assigned treatment each day for two weeks, during which they continuously wear the wearable device (e.g. Fitbit), which they regularly sync with their cellular phones. During the supplementation phase, participants keep a daily sleep diary (e.g., Consensus Sleep Diary Protocol, see Carney et al Sleep. 2012; 35 (2): 287-302). Next-morning well-being, sleep quality, and quality of life are measured by surveys. First morning after the discontinuation of the second treatment, Fitbit data and survey data on well-being were collected to assess rebound effects

### Example 8: Clinical Results of Sleep Benefits of 3-Tablet-In-1-Capsule Supplement Containing Melatonin in Humans

The supplement described in Example 4 (referred to in this Example as the “sleep supplement”) was evaluated in healthy human subjects in a randomized, double blinded, and placebo controlled clinical studies to investigate the pharmacokinetics of the sleep supplement and its effects on sleep quality parameters, according to the study design described in Example 7.

### Study Participants

22 participants with self-reported difficulty in sleeping were invited for screening. Of the 22 invited participants, two were ineligible because they had a PSQI score <5, two were too busy to take part, and two did not respond to the invite. Therefore, 16 participants were enrolled. Of the 16 participants enrolled, 10 completed the pharmacokinetic study and 13 completed the 2-week supplementation study. Of the 13 participants who completed the 2-week supplementation study, 1 excluded from the analysis per deviation from protocol due to getting ill.

### Pharmacokinetic Study Results:

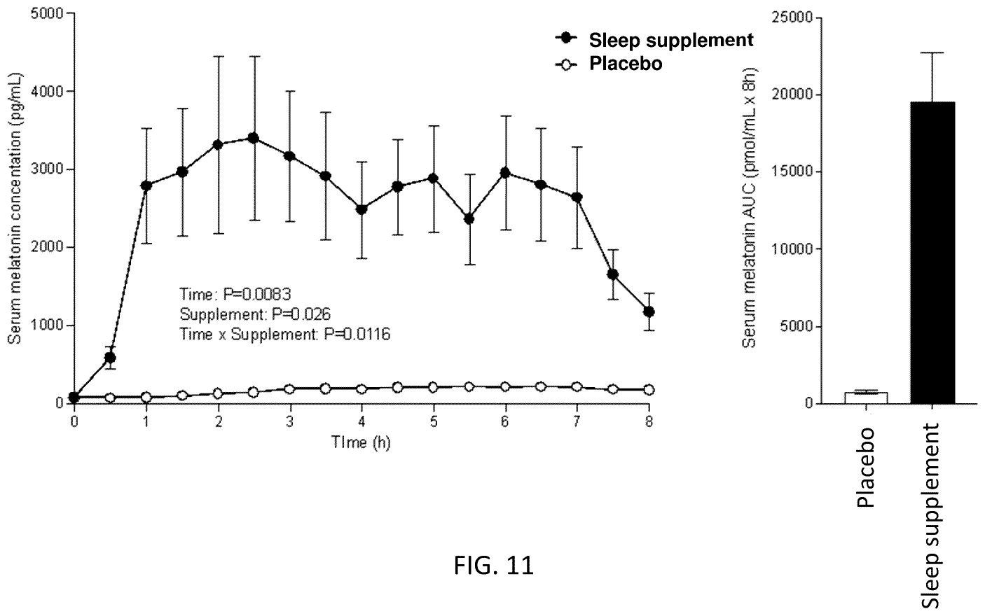

The results of the pharmacokinetic study are provided in FIGS. 11 and Table 11.

TABLE 11
Summary of pharmacokinetic study results

| | | | | | P-value |
|---|---|---|---|---|---|
| | | | | | (calculated |
| Pharmacokinetic | Sleep | | | | by pair |
| study outcome | supplement | | Placebo | | T test) |
| Mean AUC of serum | 19564 ± 3196 | | 747.6 ± 133.5 | | p < 0.0001 |
| concentration of | | | | | |
| melatonin over time | | | | | |
| Mean time to peak | 2.5 | hours | 5 | hours | p = 0.01 |
| Mean peak | 3400.7 | pg/mL | 209.3 | pg/mL | p < 0.01 |
| concentration | | | | | |

Comparing placebo and sleep supplement using a paired test showed that the sleep supplement significantly raised serum melatonin by 8.6-fold as early as 30 minutes (p<0.01) (FIG. 11A). Following placebo and sleep supplement ingestion, serum melatonin concentration increased to peaks of 209.3 and 3400.7 pg/mL (p<0.01 for mean of peak serum following placebo vs sleep supplement) after 5.5 and 2.5 hours (p=0.01 for mean of time to peak following placebo vs sleep supplement) respectively (FIG. 11, Table 12). Following peak serum melatonin concentration, there was a serum melatonin plateau over the night and tapering off from 6 hours for the sleep supplement group (FIG. 11). Therefore, the sleep supplement produced peak serum melatonin levels 3 hours faster than the placebo, with >16-fold higher peak plasma melatonin levels. An AUC analysis across 8 hours showed sleep supplement increased serum melatonin 26-fold more than placebo (p<0.0001) (FIG. 11, right).

G*Power was used to perform a post hoc power calculation to determine the sample size needed to detect the pharmacokinetic effects observed. The primary outcome for the pharmacokinetic study was melatonin AUC. The mean±SD AUC for placebo and melatonin was 748±422 and 19564±10107. Based on a 0.05a paired t-test with 80% power, this would have been detectable in 5 participants. Therefore, the sample size of the study was sufficient.

### Supplementation Study Results:

G*Power was used to perform a power calculation to determine the sample size needed to detect changes in sleep latency. The primary outcome for the supplementation study was sleep latency measured following 2-week supplementation with placebo vs. sleep supplement. A previous study that supplemented a similar does of melatonin for 4 weeks supplementation study demonstrated a mean±SD sleep onset latency of 58.9±30.3 and 20.2±17.7 minutes for placebo and melatonin respectively (Kayumov et al. Physcosom Med 2001; 63:40-8), which based on a 0.05a paired t-test with 80% power, is expected to be detected in 6 participants. It is noted that Kayumov et al. supplemented for twice as long the present study. Therefore, a sample size of at least 12 participants, i.e., twice Kayumov et al.'s sample size is believed to be sufficient for the present study.

Minutes taken to fall sleep from Pittsburgh Sleep Quality Index (PSQI) survey was used to assess sleep latency, in minutes. An analysis of covariance (ANCOVA) analysis controlling for baseline measure and sequence showed a significant effect of treatment on sleep latency (F=4.6, p<0.05). Estimated marginal means for sleep supplement and placebo were 16.2 minutes and 28.67 minutes respectively. These results suggest that the sleep supplement helped subjects fall asleep faster by 12 minutes. In other words, the sleep supplement reduced the time taken to fall asleep by 43%.

A WHO-5 survey was used to assess waking up fresh and rested, where questions are rated on a 6-point Likert scale, ranging from 0 (at no time) to 5 (all of the time). The waking up fresh and rested score was cut off at 2 (≤2 indicates waking up fresh and rested less often and >2 indicates waking up fresh and rested more often). Barnard's exact test instead of Fisher exact test was done to assess the difference as it has been suggested to be more powerful than Fisher exact test for 2×2 contingency table (Mehta, Cyrus R. and Pralay Senchaudhuri. “Conditional versus unconditional exact tests for comparing two binomials.” Cytel Software Corporation 675 (2003): 1-5; Barnard, G. A. “Significance Tests for 2×2 Tables”. Biometrika. 34.1/2 (1947): 123-138). Results showed that 11 out of 12 (92%) woke up fresh and rested more often during sleep supplement treatment compared to 6 people (50%) in placebo treatment (p=0.03).

No significant mean or distribution differences were found between groups for answering “last night sleep left me feeling” sleepy, grouchy, and tired using the Rest-Q questionnaire. No significant mean or distribution differences were found between groups for “trouble staying awake while driving, eating or engaging in social activities” as well as “having bad dreams” using PSQI.

Objective sleep quality and subjective next-morning well-being data during the first discontinuation night and the following morning were assessed for the presence of a rebound effect. The presence of a “rebound effect” was defined as a significant worsening in subjective and objective sleep quality parameters relative to an individual's worst night of baseline sleep. Participants were counted as experiencing a rebound if they meet both criteria 1 and 2:1) A significant worsening in at least one of three sleep quality parameters, as assessed by objective wearable data: a) sleep onset latency (SOL), b) total sleep time (TST), or c) number of nocturnal awakenings, and; 2) A significant worsening in next-morning well-being, as assessed by subjective REST-Q responses: 1) feeling refreshed upon awakening, 2) feeling tired upon awakening, or c) feeling grouchy upon awakening.

The results showed no significant worsening compared to the worst night of baseline and no differences between placebo and sleep supplement groups for any of the measurements, indicating no ‘rebound’ effect. When the average baseline, rather than the worst night of baseline, was used for comparison to the washout period, no differences were detected further confirming no ‘rebound’ effect.

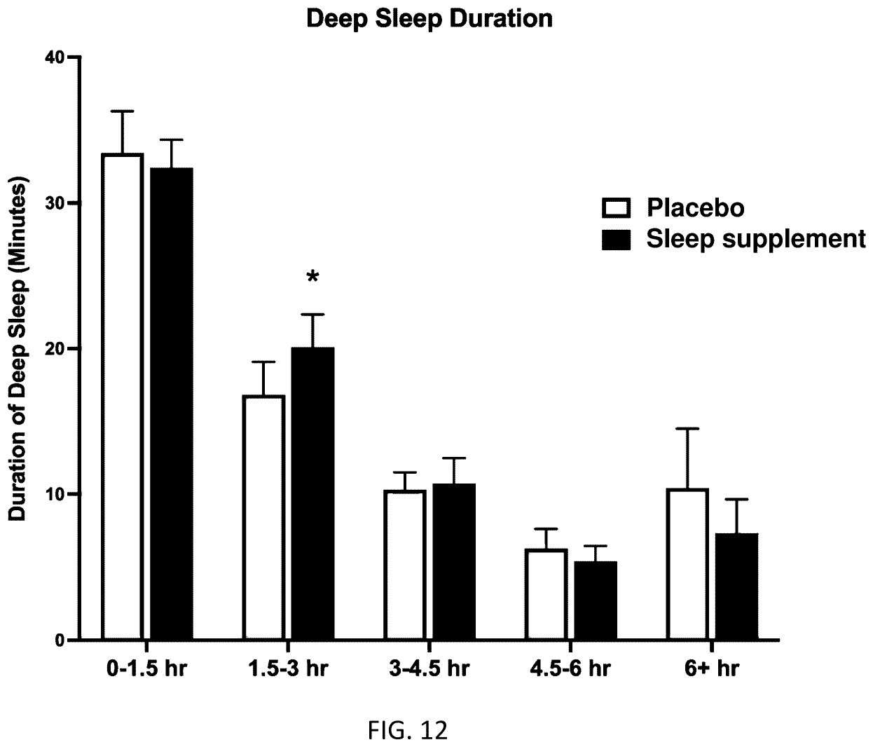

Wearable data from a Fitbit worn by participants during each supplementation period was analyzed. Total sleep averaged 8 hours and did not differ by treatment. Deep sleep averaged 77 min which also did not differ by treatment. According to the National Institutes of Health, there are 4-6 cycles per night that take about 80-100 minutes (see world wide web: nhlbi.nih.gov/health/sleep/stages-of-sleep). Therefore, the total sleep was divided to five categories for analysis: four categories of 90 minute intervals (e.g., 0-1.5 hours, 1.5-3 hours, 3-4.5 hours, and 4.5-6 hours), and one category for 6+ hours of sleep, which varied by total sleep time. As shown in FIG. 12, compared to placebo, the sleep supplement significantly improved nocturnal deep sleep 1.5-3 hours into sleep (paired t-test: p<0.05). No differences in deep sleep were found later, particularly in the closing hours of sleep. There was no difference in Rem sleep across the 5 sleep categories but for light sleep, there was a significant increase in light sleep in closing hours of sleep (6+ hours category) for sleep supplement vs placebo. Light sleep was defined as stage 1 and 2 of NREM sleep. This is important as previous research has linked waking up after slow wave sleep (also known as deep sleep) rather than light sleep to grogginess and major performance deterioration (Tassi et al, Physiol Behav. 2006; 87 (1): 177-184; Tassi P, Muzet A. Sleep Med Rev. 2000; 4 (4): 341-353. doi: 10.1053/smrv.2000.0098).

Enhanced deep sleep 1.5-3 hours into sleep (during the second sleep cycle) and lighter sleep at 6+ hours (closing hours of sleep) may explain the results observed on waking up fresh and rested after sleep supplementation indicating the sleep supplement associated with no-grogginess. Taken together, compared to placebo, the sleep supplement boosted melatonin levels, reduced the time to fall asleep, and promoted waking up feeling fresh and rested, while demonstrating no rebound effects.

### Example 9: Formulation of a 3-Tablet-In-1-Capsule Dosage Form Containing Iron for Managing Iron Deficiency

A 3-tablet-in-1-capsule dosage form is developed for supplementing iron (referred to in this Example as the “iron supplement”). The iron supplement is formulated to provide efficient iron absorption following consumption, while minimizing GI discomfort. The iron supplement is designed for consumption in an intermittent regimen (i.e., every other day in the morning). Without being bound by theory, consumption of the iron supplement in an intermittent regimen, in which consumption of the supplement is separated by an interval of about 1 day results in maximal fractional iron absorption, e.g., as compared to consumption of the supplement at a daily interval. Each tablet of the iron supplement contains iron as ferrous bisglycinate, and optionally vitamin A and/or vitamin C. The latter nutrients are included to provide antioxidant support and promote optimized iron uptake following ingestion. The first tablet is formulated for immediate release when hepcidin levels are typically at their lowest, thereby promoting optimal absorption (“tablet 1” or “instant release tablet”). The second tablet is formulated for extended release over a period of about 4 hours (“tablet 2” or “intermediate release tablet”). The aim is to enhance absorption before hepcidin levels reach a peak, while promoting reduced side effects. The third tablet is formulated for slow, continuous release over a period of about 6 hours in order to promote GI comfort throughout the day (“tablet 3” or “extended release tablet”). Tablet 2 and 3 contain Hypromellose in the tablet matrix to slow dissolution of the encapsulated active ingredients.

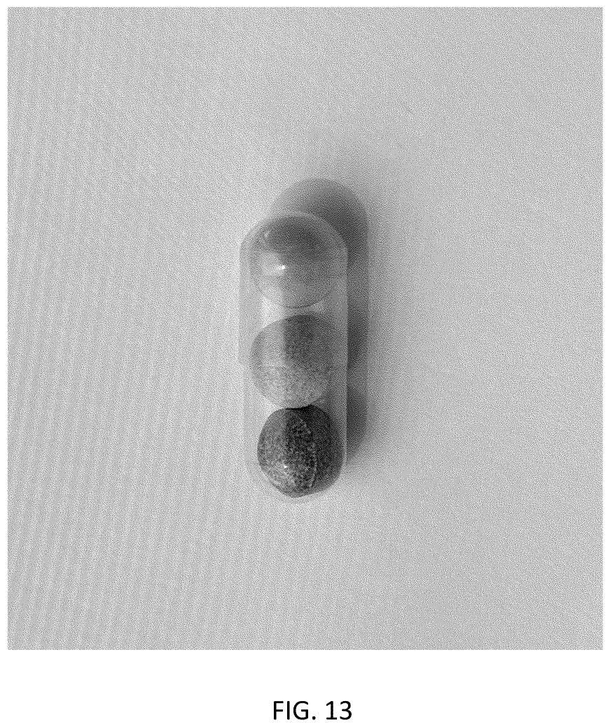

An exemplary visual image of a representative iron supplement is shown in FIG. 13. The coloring of the tablets is adjusted to improve contrast between the visual appearance of the tablets. This is done by altering the amount of tablet coating and/or the amount of active ingredient contained in the tablets.

In a representative iron supplement, the capsule is a size 00 DR capsule and contains 3 spherical (modified ball shape capsules). Three iron supplements are generated that differ in the active ingredients present in the tablets. In a first iron supplement (“iron supplement 1”), iron, vitamin C, and vitamin A are distributed equally in the three tablets. In a second iron supplement (“iron supplement 2”), each tablet contains iron, the instant release and intermediate release tablets contain vitamin C, and the extended release tablet contains vitamin A. In a third iron supplement (“iron supplement 3”), each tablet contains iron and vitamin C, but no vitamin A. The vitamin C is ascorbic acid and the vitamin A is beta-carotene. The amount of active ingredient in each of the iron supplements is provided in Table 12.

TABLE 12
Exemplary Iron Supplements

| Supplement | | | Ingredient |
|---|---|---|---|
| Name | Tablet | Ingredient | amount |
| Iron | 1-instant | Iron (bisglycinate) | 20 mg |
| supplement 1 | release | Vitamin C | 8 mg |
| | | Vitamin A | 86.66 mcg RAE |
| | | | (retinol activity |
| | | | equivalents) |
| | 2-intermediate | Iron (bisglycinate) | 20 mg |
| | release | Vitamin C | 8 mg |
| | | Vitamin A | 86.66 mcg RAE |
| | 3-extended | Iron (bisglycinate) | 20 mg |
| | release | Vitamin C | 8 mg |
| | | Vitamin A | 86.66 mcg RAE |
| Iron | 1-instant | Iron (bisglycinate) | 20 mg |
| supplement 2 | release | Vitamin C | 12 mg |
| | 2-intermediate | Iron (bisglycinate) | 20 mg |
| | release | Vitamin C | 12 mg |
| | 3-extended | Iron (bisglycinate) | 20 mg |
| | release | Vitamin A | 260 mcg RAE |
| Iron | 1-instant | Iron (bisglycinate) | 20 mg |
| supplement 3 | release | Vitamin C | 12 mg |
| | 2-intermediate | Iron (bisglycinate) | 20 mg |
| | release | Vitamin C | 12 mg |
| | 3-extended | Iron (bisglycinate) | 20 mg |
| | release | Vitamin C | 12 mg |

The dissolution profile for each tablet of the dosage form is evaluated under gastrointestinal conditions. Dissolution is measured using the method described in Example 3. The amount of iron release over time is quantified. Iron in the release media is measured by HPLC analysis.

1. A customized-release nutraceutical capsule comprising
two or more spherical tablets,
wherein each spherical tablet comprises a coating and a matrix, wherein the matrix comprises a dose of a nutraceutical, and wherein the matrix is substantially encapsulated by the coating,
wherein at least one of the spherical tablets is an extended release tablet, wherein the matrix and the coating of the extended release tablet each comprise a rate controlling polymer in an amount sufficient to release the dose over an extended duration of time, and
wherein the spherical tablets are enclosed in the capsule.

2. The capsule of claim 1, comprising 3, 4, or 5 spherical tablets.

3. (canceled)

4. The capsule of claim 2, wherein at least two of the spherical tablets are extended release tablets.

5. The capsule of claim 1, wherein the capsule comprises a cylindrical shape having a circular cross-section transverse to a longest dimension, and wherein
(i) the spherical tablets each comprise a diameter that is about 90% to about 99% of a diameter of the circular cross-section;
(ii) the spherical tablets each comprise a diameter that is substantially the same;
(iii) the spherical tablets comprise a combined volume that is about 90% to about 99% of the volume of the capsule; or
(iv) a combination of (i)-(iii).

6. The capsule of claim 1, wherein the amount of the rate controlling polymer in the matrix of the extended release tablet is about 1% to about 30% by weight of the tablet.

7-8. (canceled)

9. The capsule of claim 1, wherein the matrix of the extended release tablet further comprises a diluent, a lubricant, a glidant, and optionally a colorant.

10. The capsule of claim 1, comprising an amount of the coating of the extended release tablet, wherein the amount is about 0.5% to about 10% by weight of the tablet.

11. The capsule of claim 1, wherein the coating of the extended release tablet further comprises a flow agent and an alginate salt.

12. The capsule of claim 1, wherein at least one of the spherical tablets is an immediate release tablet, optionally wherein:
(a) the matrix of the immediate release tablet comprises a disintegrant;
(b) the matrix of the immediate release tablet further comprises a lubricant, a diluent, a glidant, and optionally a colorant;
(c) the coating of the immediate release tablet comprises a second rate controlling polymer;
(d) the capsule comprises an amount of the coating of the immediate release tablet, wherein the amount is about 0.5% to about 10% by weight of the tablet; and/or
(e) the immediate release tablet releases the dose of the nutraceutical in about 5 minutes to about 90 minutes under gastrointestinal dissolution conditions.

13-16. (canceled)

17. The capsule of claim 1, wherein the dose of the nutraceutical is about 0.05% to about 80% by weight of the tablet.

18. (canceled)

19. The capsule of claim 1, wherein the rate controlling polymer and/or the second rate controlling polymer comprises a cellulose derivative, optionally wherein the cellulose derivative is selected from hydroxypropylmethylcellulose (HPMC), hydroxypropyl cellulose (HPC), and microcrystalline cellulose (MCC).

20-22. (canceled)

23. The capsule of claim 1, wherein the nutraceutical:
(a) is selected from a vitamin, a stimulant, a mineral, a plant extract, a prebiotic, a probiotic, a postbiotic, a botanical extract, a botanical oil, a synthetic active, a botanical ingredient, a plant-based ingredient, an amino acid, a nootropic, and a combination thereof;
(b) comprises iron, vitamin C, vitamin A, a probiotic, or a combination thereof;
(c) comprises:
(i) a stimulant, optionally caffeine, guarana, or both;
(ii) caffeine, TeaCrine, L-theanine, or a combination thereof;
(iii) a botanical ingredient, optionally curcumin, ginseng, and/or rhodiola; and/or
(iv) a mineral, optionally calcium, magnesium, or both;
(d) comprises vitamin C, zinc, vitamin A, oat beta glucan, larch arabinogalactan, Euglena gracilis fermentate, elderberry, or a combination thereof;
(e) comprises a millet seed extract, an apple extract, a banana flower extract, saw palmetto, spermidine polyamines, billygoat weed extract, or a combination thereof;
(f) comprises a saffron extract, a melon juice concentrate high in superoxide dismutase (SOD), L-theanine, GABA, lemon balm extract, chamomile extract, curcumin, pomegranate extract, magnesium, carotenoids including lutein, zeaxanthin, and meso-zeaxanthin, or a combination thereof; or
(g) comprises a non-stimulant nootropic.

24-30. (canceled)

31. The capsule of claim 1, wherein the matrix of at least one of the spherical tablets further comprises a botanical oil.

32. The capsule of claim 1, wherein the nutraceutical is the same for the two or more spherical tablets, or wherein the nutraceutical is different for the two or more spherical tablets.

33. (canceled)

34. The capsule of claim 1, wherein the extended duration of time is about 1 hour to about 12 hours under gastrointestinal dissolution conditions.

35. (canceled)

36. A customized-release nutraceutical capsule, comprising
three spherical tablets comprising a first extended release tablet and a second extended release tablet,
wherein each spherical tablet comprises a coating and a matrix, wherein the matrix is substantially encapsulated by the coating, and wherein the matrix comprises a dose of melatonin, and
wherein the three spherical tablets are enclosed in the capsule.

37-54. (canceled)

55. A customized-release nutraceutical capsule, comprising:
three spherical tablets comprising a first extended release tablet, and a second extended release tablet,
wherein each spherical tablet comprises a coating and a matrix, wherein the matrix is substantially encapsulated by the coating, wherein the matrix comprises a dose of a nutraceutical selected from L-theanine, saffron, ashwagandha, and a combination thereof, and
wherein the three spherical tablets are enclosed in the capsule.

56-76. (canceled)

77. A customized-release nutraceutical capsule, comprising:
three spherical tablets comprising at least one extended release tablet;
wherein each spherical tablet comprises a coating and a matrix, wherein the matrix is substantially encapsulated by the coating, wherein the matrix comprises a dose of iron; and
wherein the three spherical tablets are enclosed in the capsule.

78-125. (canceled)

126. A pharmaceutical composition comprising the capsule of claim 1, and a pharmaceutically acceptable carrier.

127. A method for providing a health benefit to a subject, comprising administering to the subject the capsule of claim 1.

128. A method for improving sleep in a subject, comprising administering to the subject the capsule of claim 36.

129. (canceled)

130. A method for improving sleep of a subject during a nocturnal sleep period, comprising administering to the subject the capsule of claim 36 about 0.5 hours to about 2 hours prior to the nocturnal sleep period.

131-141. (canceled)

142. A method of increasing nocturnal melatonin levels in a subject, comprising administering to the subject the capsule of claim 36 about 0.5 hours to about 2 hours prior to the nocturnal sleep period.

143-155. (canceled)

156. A method for reducing stress and/or improving mood in a subject, comprising administering to the subject the capsule of claim 55.

157-158. (canceled)

159. A method for increasing, maintaining, or normalizing an iron level in a subject, comprising administering to the subject the capsule of claim 77.

160-165. (canceled)

166. A kit comprising a container comprising at least one capsule as set forth in claim 1, and a package insert comprising instructions for administering the capsule to a subject to provide one or more health benefits.

167. A kit comprising a container comprising at least one capsule as set forth in claim 36, and a package insert comprising instructions for administering the capsule to a subject to improve sleep.

168. A kit comprising a container comprising at least one capsule as set forth in claim 55, and a package insert comprising instructions for administering the capsule to a subject to reduce stress.

169. A kit comprising a container comprising at least one capsule as set forth in claim 77, and a package insert comprising instructions for administering the capsule to a subject to improve or maintain an iron level in the subject.

170-177. (canceled)

178. A method for reducing a side effect associated with a nutraceutical in a subject, comprising administering to the subject the customized-release nutraceutical capsule of claim 1,
wherein the release of the dose following the administering provides a bioavailability of the nutraceutical that results in a health benefit to the subject without substantially inducing the side effect.

179. A method for customized dosing of a nutraceutical to a subject, comprising administering to the subject the customized-release nutraceutical capsule of claim 1.

180-187. (canceled)
